(19) United States
(12) Patent Application Publication
(10) Pub. No.: US 2025/0241996 A1
(43) Pub. Date: Jul. 31, 2025

(54) COMBINATORIAL, AND ROTATIONAL COMBINATORIAL THERAPIES

(71) Applicant: Starrock Pharma Inc., Punta Gorda, FL (US)

(72) Inventor: Jorge Luis CABRERA, Port Charlotte, FL (US)

(21) Appl. No.: 19/185,055

(22) Filed: Apr. 21, 2025

Related U.S. Application Data
(63) Continuation of application No. PCT/US23/77508, filed on Oct. 23, 2023.
(60) Provisional application No. 63/380,938, filed on Oct. 25, 2022.

(51) Int. Cl. A61K 38/26; A61K 31/137; A61K 31/198; A61K 31/35; A61K 31/4985; A61K 31/702; A61K 31/7034; A61K 31/7042; A61K 38/25; A61P 3/04

(52) U.S. Cl. CPC A61K 38/26; A61K 31/137; A61K 31/198; A61K 31/35; A61K 31/4985; A61K 31/702; A61K 31/7034; A61K 31/7042; A61K 38/25; A61P 3/04

(57) ABSTRACT

Therapies and regimens for treating chronic diseases, disorders, and conditions that have a plurality of intervention targets and/or therapeutic targets are provided. Therapies include administration combinatorial regimens, and administration of rotational combinatorial regimens. The regimens comprise combinations of drugs and/or non-drug treatments.

### RELATED APPLICATION

The application is a continuation of International PCT application No. PCT/US23/77508, filed Oct. 23, 2023, and published on May 2, 2024 as Published International PCT application WO 2024/091863, to inventor Jorge Luis Cabrera, and to applicant StarRock Pharma, Inc., which claims benefit of priority to U.S. provisional application Ser. No. 63/380,938, filed Oct. 25, 2022, to inventor Jorge Luis Cabrera, and to applicant StarRock Pharma, LLC, now StarRock Pharma, Inc. This application also claims benefit of priority to U.S. provisional application Ser. No. 63/380,938.

The subject matter of each of these applications is incorporated by reference in its entirety.

### INCORPORATION BY REFERENCE OF SEQUENCE LISTING FILED ELECTRONICALLY

An electronic version of the Sequence Listing is filed herewith, the contents of which are incorporated by reference in their entirety. The electronic file was created on Apr. 18, 2025, is 58,084 bytes in size, and is titled 2701SEQ001.xml.

### FIELD

Therapies and regimens for treating chronic diseases, disorders, and conditions that have a plurality of intervention targets and/or therapeutic targets are provided. Therapies include administration combinatorial regimens, and administration of rotational combinatorial regimens. The regimens comprise combinations of drugs and/or non-drug treatments.

### BACKGROUND

In the United States, there has been an unprecedented rise in overweight and obesity in the last decade. There are more obese US adults than those who are just overweight (Deng et al., Annual Review of Pathology (2016) 11(1): 421-49). In 2008, the Journal of the American Medical Association (JAMA), reported that the obesity rate among adult Americans was estimated at 32.2% for men and 35.5% for women, which was confirmed by the CDC for 2009-2010. According to the CDC, “obesity is higher among middle-aged adults, 40-59 years old (39.5%) than among younger adults, age 20-39 (30.3%) or adults over 60 or above (35.4%) adults” (“Adult Obesity Facts,” published by the Centers for Disease Control and Prevention. Retrieved Nov. 22, 2015). Projections indicate an increase in obesity prevalence to 60% in adult men, 40% in adult women, and 25% in children by 2050 (CDC Data Brief, 2014). The overweight and obesity epidemic undermines health, was formally classified as a “disease” in June 2013 by the American Medical Association, with much controversy.

Obesity is not confined to the United States. It is a global epidemic with serious medical and financial consequences. In 1997, the WHO formally recognized obesity as a global epidemic and viewed it as one of the most serious public health problems of the 21^{st} century. Based on global trend data from 1975 to 2014, and an estimated world population of 8-9 billion, by 2025 global prevalence of obesity will reach 18% in men and 21% in women, almost a three-fold increase compared to prior to 1975. The Organization for Economic and Co-operation Development has projected an increase in obesity rates world-wide until at least 2030, especially in the United States, Mexico and England with rates reaching 47%, 39% and 35%, respectively (“Obesity Update 2017,” published by the Organisation for Economic Co-operation and Development. Retrieved 6 Oct. 2018). Once considered a problem only of high-income countries, obesity rates are rising worldwide. Globally, there are now more people who are obese than who are underweight, a trend observed in every region over the world except parts of sub-Saharan Africa and Asia (World Health Organization, Obesity and overweight. Fact sheet updated June 2016. Geneva. Retrieved 22 Sep. 2017).

There are treatments for obesity, but few that result in sustained weight loss and few that result in a loss of weight of more than 10% body weight. Hence, there is a need for treatments for obesity that result in sustained and continued weight loss to eliminate obesity.

### SUMMARY

Provided are methods for treating a disease, disorder, or condition, comprising administering and rotating combinations of drugs and/or treatments, wherein: the disease, disorder, or condition is a chronic disease, disorder, or condition that requires treatment for at least 6 months, or 9 months, or 12 months or longer, including for life; the disease, disorder, or condition has a plurality of pathways and/or targets for therapeutic intervention; each combination comprises at least two different drugs and/or treatments that target different pathways or intervention targets; at least two combinations are rotated for each cycle of treatment; and treatment comprises at least two cycles. Also provided are regimens for implementing the methods. Regimens include those for treating a disease, disorder, or condition, comprising combinations of drugs and/or treatments for use in a rotational combinatorial regimen, wherein the disease, disorder, or condition is a chronic disease, disorder, or condition that requires treatment for at least 6 months, 9 months, 12 months and longer including for life; the disease, disorder, or condition has a plurality of pathways and/or targets for therapeutic intervention; and each combination comprises at least two different drugs and/or treatments that target different pathways or intervention targets.

Combinatorial therapy/regimens administer combinations of drugs and/or treatments. The user can select a set of treatments and drugs from which subsets of combinations are selected for administration for a predetermined time, such as 1 week, several weeks, 1 month, or longer. For rotations, combinations can be selected so that all of the treatments/drugs are administered as part of at least one combination. Rotating the combinations comprises rotational combinatorial therapy. Combinations can be selected and rotated; after a selected number of combinations are administered (referred to as a cycle), the combinations can be repeated, in the same order or a different order.

Provided are methods and regimens for treating a disease, disorder, or condition, by administering rotational combinatorial therapy. Rotational combinatorial therapy employs rotation of at least two combinations.

The methods and regimens include administering a plurality of different combinations of drugs, wherein: each combination of drugs is administered for a predetermined time; each combination of drugs is rotated until all combinations are administered at least once to comprise a cycle; a cycle comprises at least two different combinations; a combination comprises at least two different drugs that target different pathways or targets involved in the disease, disorder, or condition; each combination of drugs is unique among the combinations administered in a cycle, but a drug can be part of a plurality of combinations as long as the resulting combinations are unique; the disease, disorder, or condition is a chronic disease, disorder, or condition that requires treatment for at least 6 months; and the disease, disorder, or condition is not a cancer.

Provided are methods of treating a disease, disorder, or condition, comprising administering rotational combinatorial therapy, wherein the disease, disorder, or condition has more than one therapeutic intervention target or pathway for therapeutic intervention; the disease, disorder, or condition is a chronic condition that requires treatment for at least 6 months; the rotational combinatorial therapy comprises at least two different combinations of drugs and/or treatments; the drugs and/or treatments in each combination target different pathways or targets involved in the disease, disorder, or condition; each combination is administered at least once a cycle; a cycle comprises administration of each combination at least once; a cycle comprises at least two different combinations; a cycle can be repeated a plurality of times; and at least one of the combinations comprises at least two different drugs that target different targets or pathways.

Provided are rotational combinatorial regimens for treating a disease, disorder, or condition, comprising a plurality of combinations of drugs and/or treatments for a disease, disorder, or condition, wherein: the disease, disorder, or condition has more than one therapeutic intervention target or pathway for therapeutic intervention; the disease, disorder, or condition is a chronic condition that requires treatment for at least 6 months; the rotational combinatorial therapy comprises at least two different combinations of drugs and/or treatment; the drugs and/or treatments in each combination target different pathways or targets involved in the disease, disorder, or condition; each combination is administered at least once a cycle; each cycle comprises administration of each combination at least once; a cycle comprises at least two different combinations; a cycle can be repeated a plurality of times; the cycle for each combination can be the same or a different length of time; and at least one of the combinations comprises at least two different drugs that target different targets or pathways.

Provided are methods of treating a disease, disorder, or condition, comprising administering a drug regimen comprising serially administering two or more combinations of drugs and/or treatments, wherein: the disease, disorder, or condition is a disease, disorder, or condition that has more than one therapeutic intervention target or pathway for therapeutic intervention; the disease, disorder, or condition is a chronic condition that requires treatment for at least 6 months; the drug regimen comprises at least two combinations; at least one combination in the regimen includes at least two drugs that treat at least one target or pathway involved in the disease, disorder, or condition; the at least two drugs treat different targets, pathways, and/or have different activities; a combination can include only a single drug, as long as at least one combination in the regimen has at least two drugs; the combinations are rotated so that each combination in the regimen is administered at least once per cycle; a cycle comprises administration of the regimen; and each combination is administered for a predetermined time.

Provided are regimens for rotational combinational treatment of a disease, disorder, or condition, comprising a plurality of combinations of drugs and/or treatments for a disease, disorder, or condition, wherein: the disease, disorder, or condition is a disease, disorder, or condition that has more than one therapeutic intervention target or pathway for therapeutic intervention; the disease, disorder, or condition is a chronic condition that requires treatment for at least 6 months; the drug regimen comprises at least two different combinations; at least one combination in the regimen includes at least two drugs that treat at least one target or pathway involved in the disease, disorder, or condition; the at least two drugs treat different targets, pathways, and/or have different activities; and a combination can include only a single drug, as long as at least one combination in the regimen has at least two drugs.

Provided are methods and regimens, wherein the therapy comprises at least two or three cycles of administration of combinations. The methods and regimens can comprise at least three cycles of administration of combinations.

The methods or regimens provided herein can be used for treating, preventing, or reducing the risk of a disease, disorder, or condition. Diseases, disorders, and conditions include chronic diseases, disorders, or conditions that require treatment for an extended time, such as at least 3 months, at least 6 months, at least 9 months, or at least a year.

Provided are combinatorial therapeutic methods of treating a disease, disorder, or condition, comprising administering a combination of at least 3 different drugs selected from among a plurality of drugs; wherein the disease, disorder, or condition is a chronic disease, disorder, or condition; the disease, disorder, or condition is a disease, disorder, or condition that has more than one therapeutic intervention target or pathway for therapeutic intervention; each of the plurality of drugs can treat a target or pathway involved in the disease, disorder, or condition; each of the selected drugs has a different activity from the other drugs in the combination; and the combination reduces or eliminates desensitization to one or more of the selected drugs. Included are methods and regimens where desensitization results from downregulation of a receptor agonized by a drug in a combination, when it is administered as a monotherapy, or upregulation of a receptor antagonized by a drug in a combination, when it is administered as a monotherapy. In some embodiments, the methods and regimens are those where the drugs and/or treatments in each combination are administered simultaneously, sequentially, or intermittently. In administering the drugs/treatments in a combination they can be administered together, simultaneously, serially, or intermittently. They can be administered within a period of 1,2,3,4,5,6,7,8,9, 10, 11, 12, 13, 14, 15, 16, 17, 18, 19,20,21,22,23, or 24 hours or longer, such as up to 2 or 3 days. Generally, the drugs and treatments in each combination are administered within 24 hours. The methods and regimens include those where the disease, disorder, or condition is selected from a disease, disorder, or condition that includes treatments that when used as a monotherapy, the subject becomes desensitized to the drug. The rotational methods herein can avoid desensitization. The methods and regimens can comprise at least 2, 3, or more cycles, whereby treatment lasts at least 6 months, 9 months, a year, or longer.

Among the embodiments provided herein are rotational combinatorial methods and regimens that comprise at least 3 different combinations in a cycle.

Included are methods and regimens where each combination of drugs and/or treatments comprises at least 2 different drugs or treatments that target different pathways or targets for intervention. In some embodiments, the drugs and treatments in a combination or in more than one combination can target the same pathway or target for intervention.

In all embodiments, the cancer is not among the disease, disorder, or condition that is treated for which the regimen is employed.

Provided are methods and regimens for treating a disease, disorder, or condition where the disease, disorder, or condition is a chronic infection, arthritis, Parkinson's disease, hypertension, chronic obstructive pulmonary disease (COPD), asthma, diabetes, coronary heart disease, chronic kidney disease, dementia, Alzheimer's Disease, heart failure, coronary heart disease, chronic pain, a chronic infection, and atrial fibrillation. In embodiments, the disease, disorder, or condition is a chronic disease, disorder, or condition selected from among heart disease, stroke, diabetes, arthritis, asthma, chronic obstructive pulmonary disease (COPD), hypertension, obesity, chronic infection, asthma, and Parkinson's disease. The disease, disorder, or condition can be selected from among obesity, chronic obstructive pulmonary disease (COPD), Parkinson's disease, and hypertension, or where the disease, disorder, or condition is an infection or results from an infection, such as one involving or caused by an infectious agent virus, such as a parasite, or a bacterium. Other diseases, disorders, and conditions involving infectious agents or initiated by an infection, include, for example, a disease, disorder, or condition is Lyme disease, a chronic urinary tract infection (UTI), long COVID, tuberculosis, or necrotic lesion(s) following a brown recluse spider bite.

In all embodiments of the methods and regimens herein, the disease, disorder, or condition is one wherein a monotherapy for treating the disease, disorder, or condition results in desensitization to the monotherapy; the monotherapeutic agent is administered as part of a combination; and the monotherapeutic agent is not administered continuously, but is rotated, whereby desensitization does not occur.

Among the diseases, disorders, and conditions is obesity. Provided are methods and regimens for treating obesity. Provided are combinatorial methods and regimens for treating obesity, comprising administering a combination of at least three different drugs, wherein each targets a different pathway or different target for intervention for treatment of obesity. The methods and regimens can comprise rotating combinations of drugs, to thereby provide rotational combinatorial therapy. The methods and regimens include those where the combination(s) of drugs that is/are selected mimic the effects of gastric bypass. Provided are combinatorial weight loss regimens, comprising a combination of at least three different drugs, where the activity of the drugs mimics or has the activity of a peptide whose activity is altered following gastric bypass surgery.

Provided are methods and regimens for weight loss comprising administering a combination of drugs to mimic the effects of gastric bypass surgery. Provided are such methods and regimens for treating obesity, comprising administering a combination of drugs that mimics the biological effects of gastric bypass surgery. Provided are regimens for treating obesity comprising a combination or combinations of drugs whose effects or activities mimic the biological effects of gastric bypass surgery, such as methods and regimens where biological effects of gastric bypass comprise reduced absorption and/or malabsorption of food, decreased appetite, increased satiety, increased glycogenolysis and/or lipolysis, increased insulin sensitivity, modulation of energy expenditure, and inhibition of gastric emptying. Such methods and regimens can comprise or further comprise drugs that increase growth hormone, and/or promote or result in muscle enhancement.

In accord with all of the methods and regimens provided herein, they can comprise combinations of drugs/treatments or selecting combinations of drugs and treatments, where the combinations of drugs and/or treatments are administered for a predetermined time of at least 1 week followed by administration of a different combination of drugs for a second predetermined time of at least a week, until all selected combinations of drugs are administered to complete a cycle; and repeating the same or a different cycle of combinations of drugs. For example, each combination can be administered for at least 1 week, or at least 2 weeks, or at least 3 weeks, or at least 4 weeks, or at least one month, or at least two months, or at least 3 months. Each combination can be administered for the same predetermined length of time, or each combination can be administered for a different length of time, or at least one of the combinations can be administered for a different length of time from the other combinations.

Provided are drug regimens for use for treating obesity, comprising a combination or combinations of drugs or drugs and treatments whose combined effects mimic gastric bypass, and optionally comprising additional drugs that promote or result in weight loss, wherein each combination comprises at least three different drugs that target a different pathway or intervention target involved in the etiology of obesity. The methods and regimens and uses can comprise one or more of a drug that inhibits gastric emptying selected from among one or more of GLP1, Amylin, and Pancreatic Polypeptide Therapeutic; a drug that enhances satiety comprising one or more drugs selected from among glucagon-like peptide-1 (GLP-1), peptide YY (PYY), amylin, enterostatin/gastric inhibitory peptide (GIP), cholecystokinin (CCK), and Glicentin; a drug that increases insulin release and/or sensitivity comprising one or both of GLP1 and adiponectin; a drug that modulates energy expenditure comprising a drug selected from among leptin, oxyntomodulin, and glicentin; a drug that results in muscle enhancement comprising one or more of sermorelin, tesamorelin and/or growth hormone, and testosterone; and a drug that promotes intestinal smooth muscle relaxation comprising vasoactive intestinal peptide (VIP).

The combinatorial methods, regimens and uses can be those where the drugs are selected from among drugs that have activities or effects selected from among: drugs that inhibit gastric motility, increase insulin sensitivity, accelerate glycogenolysis and/or lipolysis, reduce eating or appetite, inhibit or modulate gastric acid secretion, limit or decrease the rate of gastric emptying, enhance muscles, increase glycogenolysis, increase insulin sensitivity, enhance the body weight-lowering and/or glucose-lowering efficacy of GLP-1, medication(s) or therapy that decrease ghrelin or ghrelin-associated activation pathways, and drugs and treatments that reduce or antagonize ghrelin. Exemplary combinations of drugs include combinations of drugs that are selected from among GLP-1, Adiponectin, leptin, oxyntomodulin, peptide tyrosine-tyrosine (PYY), amylin, pancreatic peptide, enterostatin/glucose-dependent insulinotropic peptide or gastric inhibitory polypeptide (GIP,) cholecystokinin (CCK), vasoactive intestinal peptide (VIP), glicentin, human growth hormone or an active portion thereof or an analog of human growth hormone or an active portion thereof, ephedrine, caffeine, aspirin (ECA), oxyntomodulin, neuropeptide Y (NPY), antimicrobial peptide 2 (LEAP2), vaccine CYT009-GhrQb, the peptide-binding compound Nox-B11, and the ghrelin analog AZP-531 (SEQ ID NO:15). Other exemplary combinatorial methods, regimens, uses include those where the drugs that are combined comprise a GLP-1 agonist, an appetite suppressant, a thyroid hormone, a carbonic anhydrase inhibitor, an alpha-glucosidase inhibitor, a dipeptidyl peptidase-R (DPP-4) inhibitor, a sodium-glucose co-transporter 2 (SGLT2) inhibitor, a muscle enhancer, drugs that modulate energy expenditure, a GLP-1 agonist, drugs that increase gastric inhibitory polypeptide (GIP2), and mitochondrial uncouplers, such as, but not limited to those in which the drugs are selected from among one or more of dulaglide, bydureon, semaglutide, exenatide, liraglutide, phentermine, liothyronine, topiramate (carbonic anhydrase inhibitor), acarbose (alpha-glucosidase inhibitor), sitagliptin (dipeptidyl peptidase-4 (DPP-4) inhibitor), canagliflozin (sodium-glucose co-transporter 2 (SGLT2) inhibitor), dapagliflozin (SGLT2 inhibitor), sermorelin, mirabegron (beta-3 adrenergic agonist), and amylin; those where drugs in a combination or the combinations are selected from among a GLP-1 agonist, phentermine, thyroid hormone, carbonic anhydrase inhibitor, carbonic anhydrase inhibitor, alpha-glucosidase inhibitor, DPP-4 inhibitor, SGL2 inhibitor, muscle enhancer, and an appetite suppressant. The methods, regimens, and uses include those comprising a mitochondrial uncoupler, such as, for example, where the mitochondrial uncoupler is selected from among uncoupling protein 1 (UCP1), a catecholamine, and a small molecule uncoupler, such as 2,4-dinitrophenol (DNP) and BAM15 ((2-fluorophenyl){6-[(2-fluorophenyl)amino](1,2,5-oxadiazolo [3,4e]pyrazin-5-yl)}amine).

For all of the methods, regimens, and uses the combination or each combination of drugs comprises at least three drugs that target different pathways or targets for intervention. These include those in which the disease, disorder, or condition requires treatment for at least 6 months, 9 months, or 1 year, or longer, or longer and indefinitely, or for life.

Provided are methods for preventing (or reducing the risk) of desensitization to treatments for a disease, disorder, or condition, comprising administering a rotational combinatorial therapeutic regimen. Such methods employ the methods and regimens and uses described above and elsewhere herein. As described the combinations of drugs and treatments can be administered together, sequentially, intermittently, and/or within a predetermined time period, such as within up to a 24-hour period, or up to a 12-hour period, or up to a 6-hour period or less.

Exemplary of the methods, regimens and uses is a rotational combinatorial regimen for treating obesity, comprising administering a rotational combinatorial regimen comprising the following combinations: a) a first combination comprising drugs that suppress appetite, increase insulin sensitivity, accelerate glycogenolysis and/or lipolysis, enhance muscles, and inhibit gastric emptying; b) a second combination comprising drugs that inhibit gastric motility, increase insulin sensitivity, accelerate glycogenolysis and/or lipolysis, reduce eating, reduce gastric acid secretion, limit the rate of gastric emptying, promote muscle enhancement, and increase glycogenolysis; and c) a third combination comprising drugs that increase insulin sensitivity, accelerate glycogenolysis and/or lipolysis, enhance the body weight-lowering and glucose-lowering efficacy of GLP-1, and promote muscle enhancement.

Provided are rotational combinatorial regimens and methods and uses for treatment of obesity comprising at least three different combinations per cycle, wherein: a) a first combination comprises drugs that suppress appetite, increase insulin sensitivity, accelerate glycogenolysis and/or lipolysis, enhance muscles, and inhibit gastric emptying; b) a second combination comprises drugs that inhibit gastric motility, increase insulin sensitivity, accelerate glycogenolysis and/or lipolysis, reduce eating, reduce gastric acid secretion, limit the rate of gastric emptying, promote muscle enhancement, and increase glycogenolysis; and c) a third combination comprises drugs that increase insulin sensitivity, accelerate glycogenolysis and/or lipolysis, enhance the body weight-lowering and glucose-lowering efficacy of GLP-1, and promote muscle enhancement. For example, any of the methods, regimens, and uses herein can be a rotational combinatorial regimen for treating obesity, comprising administering a rotational combinatorial regimen comprising the following combinations: a) a first combination comprising drugs that inhibit gastric motility, increase insulin sensitivity, accelerate glycogenolysis and/or lipolysis, reduce eating, reduce gastric acid secretion, limit the rate of gastric emptying, promote or result in muscle enhancement, and inhibit gastric emptying; b) a second combination comprising drugs that suppress appetite, increase insulin sensitivity, accelerate glycogenolysis and lipolysis, decrease food intake and body weight, promote or result in muscle enhancement, and increase glycogenolysis; and c) a third combination comprising drugs that increase insulin sensitivity, accelerate glycogenolysis and lipolysis, enhance the body weight-lowering and glucose-lowering efficacy of GLP-1, and enhance or promote muscle. For example, a method, use, or rotational combinatorial regimen for treatment of obesity comprises at least three different combinations per cycle, wherein: a) a first combination comprises drugs that inhibit gastric motility, increase insulin sensitivity, accelerate glycogenolysis and/or lipolysis, reduce eating, reduce gastric acid secretion, limit the rate of gastric emptying, promote or result in muscle enhancement, and inhibit gastric emptying; b) a second combination that comprises drugs that suppress appetite, increase insulin sensitivity, accelerate glycogenolysis and lipolysis, decrease food intake and body weight, promote or result in muscle enhancement, and increase glycogenolysis; and c) a third combination that comprises drugs that increase insulin sensitivity, accelerate glycogenolysis and lipolysis, enhance the body weight- and lowering efficacy of GLP-1, and enhance or promote muscles. In another embodiment that is a rotational combinatorial regimen for treating obesity, the regimen, method, or use comprises the following combinations or administering a rotational combinatorial regimen comprising the following combinations: a) a first combination comprising drugs that increase insulin sensitivity, accelerate glycogenolysis and lipolysis, enhance the body weight-lowering and glucose-lowering efficacy of GLP-1, promote or result in muscle enhancement, and inhibit gastric emptying; b) a second combination comprising drugs that suppress appetite, increase insulin sensitivity, accelerate glycogenolysis and lipolysis, decrease food intake and body weight, promote or result in muscle enhancement, and increase glycogenolysis; and c) a third combination comprising drugs that inhibit gastric motility, increase insulin sensitivity, accelerate glycogenolysis and lipolysis, reduce eating and gastric acid secretion and limit the rate of gastric emptying, and promote or result in muscle enhancement. In other embodiments, the rotational combinatorial regimen, method, or use for treatment of obesity comprises at least three different combinations per cycle, wherein: a) a first combination comprises drugs that increase insulin sensitivity, accelerate glycogenolysis and lipolysis, enhance the body weight-lowering and glucose-lowering efficacy of GLP-1, promote or result in muscle enhancement, and inhibit gastric emptying; b) a second combination comprises drugs that suppress appetite, increase insulin sensitivity, accelerate glycogenolysis and lipolysis, decrease food intake and body weight, promote or result in muscle enhancement, and increase glycogenolysis; and c) a third combination comprises drugs that inhibit gastric motility, increase insulin sensitivity, accelerate glycogenolysis and lipolysis, reduce eating and gastric acid secretion and limit the rate of gastric emptying, and promote or result in muscle enhancement.

In all embodiments herein, the method or regimen or use can comprise combinations in each combination or in each cycle administered for 2-, 3-, 4-, 5-, or 6-month intervals. The intervals between or for each administration of each combination are the same or vary. In accord with the methods, regimens, and uses, at least one combination in a cycle can comprise at least three different drugs. For example, at least one combination in a cycle comprises at least four different drugs; and optionally at least one combination includes only a single drug. Exemplary methods and regimens include those set forth in any of Tables 6.1 to 6.3 and 33.

Diseases, diseases, disorders, and conditions that can be treated by the methods and regimens herein include, but are not limited to, hypertension, where, for example, the drugs in the combination are selected from among a nonselective alpha-1 antagonist, an alpha-2 agonist, a beta-1 blocker, a mixed alpha-beta antagonist, a nonselective alpha blocker, a selective α-adrenergic receptor antagonist, a selective α₁-adrenergic receptor antagonist, and a selective α₂-adrenergic receptor agonist; or wherein the drugs are selected from among one or more of a non-selective α-adrenergic receptor antagonist selected from phenoxybenzamine, phentolamine, tolazoline, and trazodone, one or more of a selective α₁-adrenergic receptor antagonist selected from among alfuzosin, doxazosin, prazosin (inverse agonist), tamsulosin, terazosin and Silodosin, and one or more of a selective α₂-adrenergic receptor agonist selected from among clonidine, guanfacine, and alpha methyl-dopa; or where the drugs are selected from among phenoxybenzamine, doxazosin, clonidine, metoprolol, Terazosin, carvedilol, and phentolamine. Exemplary of a regimen are the regimens set forth in Table 38.

In other embodiments, the method or regimen or use is one where the disease, disorder, or condition is chronic obstructive pulmonary disease (COPD). Drugs in the combinations for rotation can be selected from among bronchodilators, leukotriene inhibitors, and corticosteroids, such as for example, as set forth in Table 37.

In other embodiments, the method or regimen or use can be one where the disease, disorder, or condition is Parkinson's disease, such as one where the drugs in the combination are selected from among carbidopa and levodopa (such as that sold under the trademark Rytary® and Sinemet®), cogentin, eldepryl, comtan, and Mirapex; or where the drugs in the combinations are one or more selected from among a dopamine and/or noradrenaline transporter inhibitor, an anticholinesterase inhibitor, a peroxisome-proliferator-activated-receptor-agonist, and an iron chelator. An exemplary regimen is set forth in Table 36.

### BRIEF DESCRIPTION OF DRAWINGS

FIG. 1 depicts the gut-brain axis.

FIG. 2 depicts central and peripheral mechanisms of weight loss drugs. AGRP, agouti-related peptide (SEQ ID NO: 14); ARC, arcuate nucleus; CART, cocaine- and amphetamine regulated transcript (SEQ ID NO:16); DAT, dopamine active transporter; D1R, dopamine 1-class receptor; D2R, dopamine 2-class receptor; GABA, gamma aminobutyric acid; GABAAR, 7-aminobutyric acid type A receptor; GLP-1R, glucagon-like peptide-1 receptor (SEQ ID NO:26); MC3R, melanocortin-3 receptor; MC4R, melanocortin-4 receptor; MOPR, μ-opioid receptor; NAc, nucleus accumbens; NPY, neuropeptide Y (SEQ ID NO:35); POMC, proopiomelanocortin; VTA, ventral tegmental area; Y1R, neuropeptide Y receptor type 1; GLP-1R, glucagon-like peptide-1 receptor.

FIG. 3 depicts an exemplary therapy comprising three combinations or clusters, each containing three medications, where each combination is rotated and administered for a predetermined time.

### OUTLINE

A. DEFINITIONS
B. OVERVIEW
C. COMBINATORIAL THERAPY AND ROTATIONAL COMBINATORIAL THERAPY
   1. Identification of diseases, disorders, or conditions for treatment
      a. Disease, Disorder, or Condition with a Plurality of Known Treatments
      b. Chronic Conditions
      c. Conditions where Patients Develop a Tolerance to Treatments
   2. Development of a Combinatorial Rotational Therapy Regimen
      1) Identify known treatments/therapies for each disease state or pathways associated with each disease state
      2) Identify the pathways, mechanism of actions or targets
      3) Select treatments/therapies that activate different pathways, have different mechanism of actions or targets, and/or that are compatible with a rotational therapy
      4) Identify combinations that include at least 2 therapeutics known to activate different molecular and/or cellular pathways and design a regimen for administration of the combinations
      5) Create a regimen to administer multiple rounds of treatment, with combinations of therapeutics
D. METHODS OF TREATMENT AND USES
   1. Therapeutic Uses of the Combinatorial Therapy
      a. Combination Therapies in Cancer
      b. Combination Therapies in Pain Management
      c. Combination Therapies in Oral Contraception
      d. Combination Therapies to Treat Pathogens
      e. Combination Therapies to Treat Alzheimer's Disease
      f. Combination Therapies to Treat Hypertension
      g. Combination Therapies to Treat Parkinson's Disease
      h. Combination Therapies to Treat Chronic Obstructive Pulmonary Disease (COPD)
      i. Combination Therapies to Treat Obesity Associated Diseases and Conditions
      j. Combination Therapies to Treat Overweight and Obesity
   2. Administration
E. COMBINATORIAL AND ROTATIONAL COMBINATORIAL THERAPY FOR WEIGHT LOSS
   1. Limitations of Existing Treatments for Weight Loss
   2. Obesity and the Challenges of Treatment
   3. Pharmacological Treatments
      a. Amphetamines (e.g., phentermine-topiramate)
      b. Lipase inhibitors (e.g., Orlistat)
      c. Serotonergic agonists—Neuromodulators (e.g., lorcaserin)
      d. Bupropion/Naltrexone (sold under the trademark Contrave®)
      e. Glucagon-like peptide-1 receptor (GLP1R) agonists
      f. Mitochondrial uncouplers
      g. Thyroid hormones
      h Drug cocktails
      i. Cannabinoid receptor antagonists
      j. GIPR agonists and GIPR/GLP1R combination agonists
      k. GLP1R/glucagon dual agonists
      l. Summary
   4. Surgical Treatments
F. MODIFICATIONS AND ENHANCEMENTS OF PHARMACOLOGICAL WEIGHT LOSS TREATMENTS TO IMPROVE CLINICAL OUTCOMES
   1. Developing Combination and Rotational Combinatorial Therapies for Weight Loss
   2. Pathways to Target for Weight Loss
      Glucagon like peptide-1
      Adiponectin
      Leptin
      Oxyntomodulin
      Sermorelin acetate
      Peptide YY (PYY)
      Amylin
      Tesamorelin
      Pancreatic polypeptide
      Gastric Inhibitory Polypeptide
      Enterostatin
      Ghrelin
      Cholecystokinin (CCK)
      Vasoactive intestinal peptide
      Glicentin
      GDF15
      Summary
      a. Therapeutic Combinations and Regimens
      b. Combination Drug Therapy to Gastric Bypass
G. PHARMACEUTICAL PRODUCTION, COMPOSITIONS, AND FORMULATIONS
   1. Formulation and Administration of the Combinatorial Therapy
   2. Dosage Forms
   3. Dosage and Administration
   4. Dosage and Administration for Treating Obesity and Overweight
   5. Routes of Administration of the Combinations
   6. Administration of Combinatorial Treatments for Improved Weight Loss
   7. Articles of Manufacture and Kits
H. METHODS OF ASSESSING ACTIVITY, BIOAVAILABILITY AND PHARMACOKINETICS
   Pharmacokinetics and tolerability
I. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, therapies used interchangeably with treatments include drugs and can include other non-drug treatments. Thus, treatments include drugs (or medications) and non-drug treatments, such as, for example, surgery.

As used herein, drugs include any administered therapeutic and is used interchangeably with medications.

As used herein, a therapeutic agent is used interchangeably with therapeutic and includes, but is not limited to: conventional drugs and drug therapies; vaccines; pharmaceutical medications; pharmaceuticals; homeopathic medications; peptides; protein therapeutics; radio-protectants; radiation therapy; and chemotherapeutics. A therapeutic agent can effect amelioration of symptoms of a disease, disorder, and/or condition or can prevent or reduce the risk of developing a disease, disorder, or condition, or reduce the severity of disease, disorder, or condition. The combinations when used for the combinatorial therapy as described herein include at least 3 treatments, such as three different therapeutics. For combinatorial rotational therapy combinations that are rotated, the combinations can include 2 treatments in a combination or combinations of treatments. Some of the rotations can include a single drug or therapy, as long as one or more others rotational combinations include at least two. Generally, each rotated combination includes at least two different drugs or treatments.

As used herein, combinatorial therapy or combinatorial protocol or regimen refers to combinations of treatments for a disease, disorder, or condition from among a plurality of treatments, at least 2 and generally at least 3, where each treatment acts on or interacts with or modulates a different target molecule and/or target pathway involved in or that mediates the disease, disorder, or condition.

As used herein, a rotational combinatorial therapy is used interchangeably with combinatorial rotational treatment, or rotational combinatorial therapy, or rotational combination therapy, or rotational combination treatment or CRT, or grammatical variations thereof. A rotational combinatorial therapy or protocol or regimen includes at least two different therapeutic combinations at least one of which, and generally each of which, includes at least two therapeutics or therapies known to activate different molecular and/or cellular pathways or targets involved in a disease, disorder, or condition. Combinatorial rotational therapy, thus, is a combinatorial therapy protocol that includes a plurality of rounds of treatment with different combinations of treatments or with the same combination but not successively. The different combinations are therapeutics are administered for a predetermined time, and then rotated for another predetermined time. The number of rotations of combinations can be two, three, four, five or more, and can be administered for weeks, months, years, and indefinitely depending upon the disease, disorder, or condition. The predetermined time can be days or weeks or months, generally is about 2-6 weeks. Each predetermined time is not necessarily the same.

As used herein, a rotational combinatorial pharmacological treatment (also referred to a rotational combinatorial therapy refers to a combinatorial therapy or treatment with pharmaceuticals or medications or drugs or treatments in the combinations where combination are rotated. The rotational combinatorial pharmacological treatment includes a plurality of combinations of a plurality of pharmaceuticals or medications or drugs that are rotated in accord with a rotational regimen or protocol. The rotational regimen generally is employed to prevent desensitization to a particular drug. A rotational combinatorial regimen herein includes two or more different therapies or therapeutics (e.g., combinations of therapies and/or therapeutics) that are administered such that the combinations of the therapies are rotated for a plurality of rounds of treatment. Where there is a plurality of rounds of treatment, at least one round can include only a single treatment. Generally, for rotational combinatorial protocols, the rounds include combinations of at least two different treatments, where each targets a different pathway involved in the disease, disorder, or condition.

As used herein, rotate or rotational or grammatical variations thereof include an exchange of one treatment for another. A combinatorial rotation refers to changing one therapeutic combination to another, which has the effect of improving the therapeutic response, such as by avoiding desensitization to a therapeutic of combination thereof. This also can reduce adverse side effects, and/or the severity of adverse side effects. In examples herein, a rotation includes a regimen in which a first combination of more than one therapeutics is administered, and the therapy is rotated (or switched) to a second combination one or more one therapeutic, and, optionally, the therapy is rotated (or switched) to a third combination of more than one therapeutics. Generally, the rotations occur according to a predetermined time schedule, or in view of a physician's judgement, such as based on an observation that a combination has reduced effect. In some examples, the therapies are rotated more than three times, such as more than four times, more than five times, more than six times, more than seven times, more than eight times, more than nine times, or more. In some instances, such as chronic diseases, disorders, and conditions, the rotation of therapies continues for up to life.

As used herein, a regimen is used interchangeably with protocol and refers to a course of medical treatment. In some examples the regimen is designed to improve or preserve the health of the patient or to attain a particular result. A therapeutic regimen or protocol includes the timing for administration of the therapeutics, timing for cessation of therapeutics, therapeutic dosage(s), the particular therapeutics that will be included in particular combinations, and other components of experimental design, such as how therapeutics will be administered. A protocol can include provisions for how the protocol can be modified, such as, for example, in the case of receptor downregulation or an increase in adverse side effects.

As used herein, a cycle is generally a series of events that are repeated regularly in the same order. A cycle, with reference to a rotational combinatorial regimen, refers to the repeated administration of combinations of drugs, where a combination of drugs (or a drug) is administered for a period of time followed by another combination of drugs, and so on until all combinations in a cycle of treatment are administered. The cycle then can be repeated; generally, the same combinations of drugs or treatments are administered. It, however, is within the discretion of the physician to change the drugs/treatments in accord response(s) of the treated subject, such as to improve therapeutic effectiveness, eliminate or reduce desensitization, and/or for other reasons, such as to reduce adverse side effects. In accord with a rotational regimen, a combination can include a single drug, as long as at least one combination in the cycle includes two or more drugs or treatments.

As used herein, disease or disorder or condition refers to a pathological or undesirable or undesired state in an organism resulting from a cause or condition including, but not limited to, infections, acquired conditions, and genetic conditions, and that is characterized by identifiable symptoms. Obesity and overweight are conditions characterized by excess body weight.

As used herein, a chronic condition is one that is expected, based on physician's experience and knowledge in the art to last more than 6 months, and generally more than a year. In particular, it is a condition that requires treatment for at least 6 months, and can require at 1 year or more, including for life.

As used herein, treating a subject with a disease, disorder, or condition means that a drug, therapeutic, composition, combination or other therapy is administered to the subject and the subject's symptoms or manifestations of the disease or conditions are partially or totally ameliorated, or remain static (do not worsen) following treatment. In examples, treating a subject with a disease, disorder, or condition includes treating a subject with a rotational combinatorial therapy described herein.

As used herein, a pathway refers to a biological pathway that is involved in or mediates disease, disorder, or condition. A pathway can be targeted via a component of the pathway, such as a receptor or ligand involved in the pathway. Depending upon the disease, disorder, or condition and the role of the pathway in the disease, disorder, or condition, the targets can be antagonized or agonized.

As used herein, a “combination” refers to any association between two or among more items. The association can be spatial or refer to the use of the two or more items for a common purpose. A combination for therapeutic use(s), such as a rotational combinatorial therapy provided herein, includes more than one therapeutic (i.e., medication), such as two, three, four, five, or more therapeutics, but generally includes up to five therapeutics in the combination. The rotational combinatorial therapy provided herein includes a regimen in which two, three, four, five or more combinations are rotated. The combinations for rotation in the regimen provided herein can contain different therapeutics or can contain some of the same therapeutics, but do not contain all of the same therapeutics in the combinations. A “combination” is used herein interchangeably with a cluster, which includes two or more treatments, such as two or more medications. Each combination can be administered as a therapy, or can be rotated and administered for a predetermined time, which can be shortened or lengthened according to the judgement of a physician. When not rotated each combination generally includes at least 3 different treatments

As used herein, treatment means any manner in which the symptoms of a condition, disorder or disease are ameliorated or otherwise beneficially altered. Hence, treatment encompasses prophylaxis, therapy and/or cure. Treatment also encompasses any pharmaceutical use of the compositions and combinations herein. Treatment also encompasses any pharmaceutical use of a combination of pharmaceutical and non-pharmaceutical therapeutics and compositions provided herein. Treatment encompasses a rotational combinatorial therapy described herein, in which a combination of therapeutics is administered in accord with any regimen described herein.

As used herein, amelioration of the symptoms of a particular disease or disorder by a treatment, such as by administration of a pharmaceutical composition or other therapeutic(s), refers to any lessening, whether permanent or temporary, lasting or transient, of the symptoms that can be attributed to or associated with administration of the composition or therapeutic(s). In some examples, amelioration of symptoms includes amelioration of side effects of the composition or therapeutic(s). In general, amelioration of adverse symptoms of a disease or disorder is a decrease in number or severity of the symptoms of the disease compared to the number or severity of the symptoms of the disease prior to treatment. In some examples, amelioration of a disease or condition can include a decrease in levels of a biomarker of the disease, such that the biomarker level is decreased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98% or 99% compared to the biomarker level prior to treatment. In other examples, symptoms of the disease are decreased, such as the amount or severity of symptoms that are decreased by at least or about 1%, 2%, 3%, 4%, 5%, 6%, 7%, 8%, 9%, 10%, 20%, 30%, 40%, 50%, 60%, 70%, 80%, 90%, 91%, 92%, 93%, 94%, 95%, 96%, 97%, 98% or 99% compared to the amount or severity of symptoms level prior to treatment.

As used herein, incidence refers to how often an event occurs. For example, adverse side effect incidence refers to how often adverse side effects occur after administration of a therapeutic, for example the frequency of adverse side effects after administration of a combination therapy provided herein. For example, an adverse side effect(s) can occur 10%, 20%, 30%, 40%, 50%, 60%, 70%, 80%, 90%, 91%, 92%, 93%, 94%, 95%, 96%, 97%, 98%, 99%, or 100% of the time after administration of a rotational combinatorial therapy provided herein. In examples herein, adverse side effects incidence decreases when multiple therapeutics are formulated in a combination and administered in accord with a rotational regimen provided herein compared to a monotherapy or compared to a combination that is not rotated (i.e., is continuously administered or is administered over a longer period of time). For example, the incidence of an adverse side effect(s) can decrease 10%, 20%, 30%, 40%, 50%, 60%, 70%, 80%, 90%, 91%, 92%, 93%, 94%, 95%, 96%, 97%, 98%, 99%, or 100% multiple therapeutics are formulated in a combination and administered in accord with a rotational regimen provided herein, compared to a monotherapy or compared to a combination that is not rotated.

As used herein, severity refers to degree of illness or symptoms of the illness or side effects of the illness or side effects of treatment(s) for the illness manifested by a patient. The skilled physician can assess the severity of an illness or progression of the severity of an illness or the severity of side effects of the illness or side effects of treatments with therapeutics for the illness based on the knowledge in the field of medicine and pharmacology. The skilled physician or scientist can assess the adverse side effects associated with administration of therapeutics for a disease, disorder, or condition; for example, as severe (e.g., adverse side effects requiring medical care), or as mild (e.g., adverse side effects not requiring medical intervention or treatment). For example, adverse side effects can be classified by severity using medical diagnosis codes used in clinical assessment and tracking, such as classification of severity as minor, moderate, major, and extreme. In some examples herein, adverse side effects following administration of the rotational combinatorial therapy provided herein are less severe than adverse side effects following administration of monotherapy or following administration of a combination therapy that is not rotated.

As used herein, “adverse effect” or “adverse side effect” refers to a harmful, deleterious and/or undesired effect of administering a medication or drug. Side effects or adverse effects are graded based on toxicity and various toxicity scales exist providing definitions for each grade. Exemplary of such scales are toxicity scales of the National Cancer Institute Common Toxicity Criteria version 2.0, the World Health Organization or Common Terminology Criteria for Adverse Events (CTCAE) scale. Generally, the scale is as follows: Grade 1=mild side effects; Grade 2=moderate side effects; Grade 3=Severe side effects; Grade 4=Life Threatening or Disabling side-effects; Grade 5=Fatal. Assigning grades of severity is within the experience of a physician or other health care professional.

As used herein, a dose-limiting toxicity (DLT) refers to the dose of a drug that produces side effects severe enough to prevent larger doses being given. It is within the level of skill of a skilled physician to assign or determine a DLT depending on the treatment protocol, the administered treatment, the disease to be treated, the dosage regime, and the particular patient to be treated. Generally, for the treatments, protocols, and regimens provided herein, a DLT is the dose of a drug results in an adverse event or side effect that on the toxicity scale of at least an ongoing or persistent Grade 2 toxicity that fails to resolve over the course of treatment and that limits the patient's ability to comply with the protocol therapy. As part of rotational therapy, a drug that results in such side effects can be administered at a lower dose, since the combinations of drugs/treatments can act synergistically, or the drug can be discontinued, and optionally replaced with another drug with similar activity or effect.

As used herein, prevention or prophylaxis refers to methods in which the risk of developing disease or condition is reduced. Prophylaxis includes reduction in the risk of developing a disease or condition and/or a prevention of worsening of symptoms or progression of a disease or reduction in the risk of worsening of symptoms or progression of a disease and/or a prevention of worsening of symptoms or progression of a disease or reduction in the risk of worsening of symptoms or progression of a disease. Prevention includes inhibition or avoidance of a disease, disorder, or condition by administration of a rotational combination therapy provided herein.

As used herein, a “prophylactically effective amount” or a “prophylactically effective dose” refers to the quantity of an agent, compound, material, or composition containing a compound, which, when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose, and can occur only after administration of a series of doses, such as a rotation of combinations of therapeutics. Thus, a prophylactically effective amount can be administered in one or more administrations. A prophylactically effective amount of a combination therapy as described herein can be lower than a prophylactically effective amount of a monotherapy. A prophylactically effective amount of a rotational combinatorial therapy as described herein can be lower than a prophylactically effective amount of a monotherapy or a combination therapy that is not rotated.

As used herein, an “effective amount” of a compound or composition for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. A measure of effective amount or dosage is the median effective dose (ED₅₀), which is the dose that produces a response that is fifty percent of the maximum obtainable response. As described herein, an effective amount of an individual therapeutic or a therapeutic combination is the amount required to achieve a desired amelioration of symptoms. A rotational combination therapy as can employ a lower effective amount of drug compared to a than a monotherapy or a combination therapy that is not rotated.

As used herein, a therapeutically effective amount or a therapeutically effective dose refers to the quantity of an agent, compound, material, or composition containing a compound that is at least sufficient to produce a therapeutic effect following administration to a subject. Hence, it is the quantity necessary for preventing, curing, ameliorating, arresting, or partially arresting a symptom of a disease or disorder. A therapeutically effective amount or dose can reference the amount or dose of a particular therapeutic in a combination, and/or can reference the amount or dose of the combination of therapeutics in a combination.

As used herein, “therapeutic efficacy” refers to the ability of an agent, compound, material, composition, or combination of agents, compounds, materials, or compositions containing a compound to produce a therapeutic effect in a subject to whom the agent, compound, material, composition, or combination of agents, compounds, materials or compositions containing a compound has been administered. For example, the therapeutic efficacy can refer to the therapeutic efficacy of a combination of therapeutics or a plurality of combinations, such as the combination(s) for use in a rotational combinatorial therapy.

As used herein, a standard dosage of a therapeutic is the dosage for formulation or administration that is approved by applicable regulatory agencies in the field, for example the U.S. Food and Drug Administration (FDA), such as the FDA Data Standards Advisory Board. In some examples a standard dosage includes an amount (i.e., mass) of a therapeutic (i.e., drug) for formulation. A standard dosage also can include a dosage regimen for administering the standard dosage (i.e., biweekly dosing). A standard dosage as approved by a regulatory agency or body also can be a range of dosages or the dosage can vary based on characteristics of the patient to whom the therapeutic will be administered. For example, the dosage can vary depending on the weight of the patient, or the severity of the disease or condition for which the therapeutic(s) is/are administered. A standard dosage also can be defined by pooled analysis using scientific studies of real-world data, where efficacy is established for various doses, and the dosage is established as a dosage with a particular efficacy. When standard dosage is established, efficacy, adverse side effects, and other factors are considered. An individual therapeutic (i.e., drug) in a combination for a rotational combinatorial therapy provided herein can be formulated in a standard dosage. In some examples, an individual therapeutic (i.e., drug) in a combination for a rotational combinatorial therapy provided herein is initially formulated in a standard dosage and the dosage is modified from the standard dosage for subsequent administrations. For example, the dosage of the individual therapeutic is formulated in a lower or higher dosage compared to a standard dosage.

As used herein, a peptide refers to a polypeptide that is from 2 to 300 amino acids in length. Peptides include therapeutic peptides that are administered for treatment of a disease, disorder, or condition. Peptides that are administered for therapeutic treatment are administered in an amount effective to elicit a therapeutic effect. Peptides for treatment can be administered at a dosage wherein the resulting circulating or accumulating levels of the peptide mimic normal levels or the peptide levels of a person who is not afflicted by the disease or condition. A therapeutic peptide or peptides also can be administered at a dosage so that the circulating level of the peptide is similar or the same of an endogenous peptide that elicits a therapeutic effect. For example, as described herein, gastric bypass is the most effective treatment to effect sustained and significant weight loss. It is known in the art that gastric bypass, not only reduces the size of the stomach to thereby limit food intake; it also results in changes in peptide hormones involved in regulating food intake and effects (see, e.g., Beckman et al. (2010) J Am Diet Assoc. 110:571-584, doi: 10.1016/j.jada.2009.12.023). In accord with the combinatorial therapeutic methods and regimens provided herein, peptides (or agonists), such as glucagon-like peptide-1 (GLP-1) (SEQ ID NO:1), peptide tyrosine-tyrosine (PYY) (SEQ ID NO:6), and leptin (SEQ ID NO:3) that increase after gastric bypass can be exogenously administered to overweight or obese patients to mimic peptide levels and patterns of expression observed after gastric bypass to effect weight loss. Antagonists of peptides that decrease after bypass can be administered.

As used herein, parenteral and parenterally refer to administration of an agent via any route other than oral administration. Parenteral includes the injection of a dosage form into the body by a sterile syringe or some other mechanical device, such as, for example an infusion pump. For example, parenteral administration includes injection (i.e., subcutaneous, intramuscular, and/or intravenous injection), infusion, implantation, intraperitoneal routes of administration, and any other mode of delivery other than ingestion to any site in or on the body of a subject.

As used herein, monotherapy refers to the use of a single therapeutic (i.e., drug or medication) to treat a particular disorder or disease. A monotherapy can include continued treatment, such as for months or years, with a single therapeutic.

As used herein, combination therapy refers to the administration of two or more different therapeutics or other treatment(s), such as radiation and surgery. Multiple therapeutic agents or treatments in the combination therapy can be provided and/or administered separately, sequentially, intermittently, simultaneously, or provided in a single composition. Generally, the two or more different therapeutics or other treatment(s) in a combination therapy are administered together or separately, or intermittently. For example, the treatments can be within 6 hours, 8 hours, 10 hours, 12 hours, 18 hours, 24 hours, 36 hours, 48 hours, 72 hours, 4 days, 5 days, 6 days, or 1 week of the other treatments in the combination. The timing and order of administration depends upon the disease, disorder, or condition, and is within the judgement of a physician.

As used herein, an orexigenic pathway is one that promotes appetite. Orexigenic pathways can be activated by increased AMP-activated protein kinase (AMPK) activity by ghrelin to promote appetite (orexia). The peptides orexin A (SEQ ID NO:36), neuropeptide Y (NPY) (SEQ ID NO: 35) and ghrelin (SEQ ID NO:23) stimulate appetite and act through orexigenic pathway(s).

As used herein, a receptor is a molecule that has an affinity for a particular ligand. For example, a receptor is a protein that specifically binds a signal molecule and then initiates a response. Receptors can be naturally-occurring or synthetic molecules. Receptors also can be referred to in the art as anti-ligands. In some examples, multiple receptor types are involved in a signaling pathway. In the context of pharmacology, receptors include macromolecules in the membrane or inside the cell that specifically (chemically) bind a ligand (drug).

As used herein, downregulate or downregulation refers to reducing or suppressing the body's response to specific stimuli. Receptor downregulation is characterized by a decrease in total receptor number in the cell. Receptor downregulation can be caused by long-term exposure to agonists. Cells undergo receptor downregulation as a reversible process of adaptation, or desensitization, whereby a prolonged exposure to a stimulus decreases the cellular response to that level of exposure. In response to continuous drug exposure, receptor downregulation occurs and can decrease the drug efficacy due. Rotational and/or combinatorial drug therapy described herein decreases receptor downregulation due to the activation of multiple pathways as opposed to the one activated pathways in monotherapy. Rotational and/or combinatorial drug therapy described herein also can overcome the decreased therapeutic response due to receptor downregulation by rotating to a next combination with therapeutics that activate a pathway that is not downregulated.

As used herein, body mass index (BMI) is a value derived from the mass (weight) and height of a person. The BMI is defined as the body mass divided by the square of the body height, and is expressed in units of kg/m², resulting from mass in kilograms and height in meters. BMI value is used to categorize a person as underweight (under 18.5 kg/m2), normal weight (18.5 to 24.9), overweight (25 to 29.9), or obese (30 or more) based on tissue mass (muscle, fat, and bone) and height. BMI under 20 and over 25 have been associated with higher all-causes mortality, with the risk increasing with distance from the 20-25 range.

As used herein, weight loss means a decrease in body mass of a person. Weight loss includes overall loss of fat, muscle and water compared to a pre-set time point. In examples herein, weight loss is assessed after treatment with a rotational combinatorial therapy compared to weight prior to treatment.

As used herein, off label use means the U.S. Food and Drug Administration (FDA) has approved the drug for a particular use and the approved drug is used for a different therapeutic use. The FDA has determined the benefits of using the drug for a particular use outweigh the potential risks, but has not assessed the risk or benefits of the drug for the off-label use.

As used herein, potency is the drug concentration required to produce an effect of a specified intensity. Potency generally is calculated as the concentration (or dose) required to produce 50% of the drug's maximal effect (EC>50). EC50 is used to express the potency in in vitro studies and also is the dose required for an individual to experience 50% of the maximum effect, and median effective dose or ED50 is used to measure a drug's potency in a population (i.e., animal studies or human populations). ED50 is the dose that produces the desired effect in 50% of the population. As applied to clinical settings, potency can indicate the dose of the drug, whereas efficacy can indicate the magnitude of the response (regardless of the dose).

As used herein, an “adverse effect,” or “side effect,” or “adverse event,” or “adverse side effect,” refers to a harmful, deleterious and/or undesired effect associated with administering a therapeutic agent. For example, side effects associated with the administration of a monotherapy, such as continuous administration of a monotherapy over an extended period of time. Such adverse side effects include, for example, headaches, nausea, diarrhea, heartburn, gas, constipation, dry mouth, dizziness, increased blood pressure, increased heart rate, restlessness, drug dependence, abuse, and withdrawal symptoms. Other serious adverse effects include infections, such as tuberculosis, and other infections caused by viruses, fungi and bacteria, including upper respiratory infections, as well as dermatological and dermal toxicity, such as rash. Thus, “adverse effect” or “side effect” refers to a harmful, deleterious and/or undesired effect of administering a therapeutic agent. Side effects or adverse effects are graded on toxicity, and various toxicity scales exist, providing definitions for each grade. Examples of such scales are toxicity scales of the National Cancer Institute Common Toxicity Criteria version 2.0, and the World Health Organization or Common Terminology Criteria for Adverse Events (CTCAE) scale. Assigning grades of severity is within the skill of an experienced physician or other health care professional. The severity of symptoms can be quantified using the NCI Common Terminology Criteria for Adverse Events (CTCAE) grading system. The CTCAE is a descriptive terminology used for Adverse Event (AE) reporting. The grading (severity) scale is provided for each AE term. The CTCAE displays Grades 1 through 5, with clinical descriptions for severity for each adverse event based on the following general guideline: Grade 1 (Mild AE); Grade 2 (Moderate AE); Grade 3 (Severe AE); Grade 4 (Life-threatening or disabling AE); and Grade 5 (Death related to AE/fatal).

As used herein, serum level refers to the amount of a therapeutic in blood plasma. Serum level can be used to assess whether the amount of the therapeutic administered is safe and/or effective. Therapeutic drug monitoring of the concentration of mediation(s) in body fluids such as blood serum can be used during treatment and/or for diagnostic purposes. Assessing drug serum levels can be used, for example, to avoid drug toxicity, to determine if drug serum level is toxically high; to adjust dose, such as, for example, after reaching a steady state, to determine if the loading dose was adequate, and/or to predict a patient's dosing requirements; and for monitoring patient compliance, diagnosing undertreatment (i.e., when drug dose can be increased), and diagnosing ineffective treatment.

As used herein, comorbidity refers to the presence of one or more additional conditions co-occurring (that is, concomitant or concurrent) with a primary condition. Comorbidity indicates that the one or more conditions occur simultaneously with the primary condition or as a result of the primary condition. For example, hypertension can be a comorbidity of obesity, where a subject has hypertension as a result of the obese state. In some examples, complications of the primary condition and the comorbidity are the same or overlap.

As used herein, disease or disorder refers to a pathological condition in an organism resulting from cause or condition including, but not limited to, infections, acquired conditions, genetic conditions, and characterized by identifiable symptoms. Diseases and disorders of interest herein are those that can be treated with a rotation of pharmaceutical agents. Diseases and disorders also include those where treatment of the disease or disorder is impaired due to downregulation of receptors or drug targets, leading to a decreased efficacy of the therapeutics. Of particular interest herein are those disorders where treatment is not effected due to resistance to the expected efficacy of the therapeutic molecule or drug or peptide.

As used herein, “chronic” is used to characterize the duration of a disease, disorder, or condition. For example, a chronic condition is a condition that is prolonged and requires ongoing medical intervention or limit activities of daily living or both. A chronic condition generally is a condition that lasts more than 3 months, 6 months, 1 year or more, or that is terminal. A chronic condition can persist when the affected individual receives treatment or therapeutics, and during the amelioration of signs or symptoms of the condition. Chronic conditions include chronic diseases or disorders. Chronic conditions can be characterized by one or more of etiology, duration, onset, recurrence/pattern, prognosis, sequelae, diagnosis, severity and prevalence (see e.g., O'Halloran et al., Family Practice, (2004) 21(4): 381-386).

As used herein, “obesity” refers to a condition in which the subject has abnormal or excessive fat accumulation, such as a body mass index of greater than 30. Obesity can be a health risk. Obesity can be caused by a multitude of factors, including genetic and environmental factors. Patients with obesity can have other comorbidities, such as, for example, high blood pressure, type 2 diabetes, cardiovascular disease, high cholesterol, and others.

As used herein, “overweight” refers to a condition in which the subject has extra fat accumulation or weight that is higher than typical, with a body mass index that is, for example, greater than 25. Overweight also can be assessed by other methods known in the art, such as by assessing waist and/or hip circumference, subcutaneous fat thickness, percent of fat or muscle compared to total body composition, overall body weight, and similar assessments. Other metrics can be used to determine whether a subject is overweight, such as a waist to hip ratio of greater than, for example, 1.0. Overweight subjects are at increased risk various comorbidities, including high blood pressure, high cholesterol, COPD, type II diabetes, and others. Overweight can be caused by a multitude of factors, including genetic and environmental factors.

As used herein, a “pharmacokinetic property” refers to a property related to the action of a drug or agent, such as a therapeutic peptide, in the body and in particular the rate at which drugs are absorbed, distributed, metabolized, and eliminated by the body. Pharmacokinetics can be assessed by various parameters. These include, but are not limited to, clearance, volume of distribution and serum half-life. Pharmacokinetic properties of peptides can be assessed using methods well known in the art, such as, for example, administering the peptide to a human or animal model and assessing the amount of the peptide in the body (e.g., in the bloodstream) at various time points. The various parameters, such as clearance, volume of distribution and serum half-life, are assessed using calculations well known in the art and described herein.

As used herein, “improved pharmacokinetic properties” refers to a desirable change in a pharmacokinetic property of a peptide or combination of peptides or combination of therapeutics, such as peptides in a pharmaceutical composition for administration with the timing or regimen set forth herein, compared to, for example, a peptide or therapeutic administered continuously or once. The change can be an increase or a decrease.

As used herein, “synergistic effect” or “synergy” or grammatical versions thereof refers to a larger therapeutic effect of the combined treatment compared to the effect predicted from the sum of each therapeutic alone. For example, when the action of one drug is increased when administered in the presence of another drug.

As used herein, plasma half-life (t½) refers the elimination half-life of a peptide(s) or therapeutic(s) or combinations thereof or the time at which the plasma concentration of the administered peptide(s) or therapeutic(s) or combinations thereof has reached one half of its initial or maximal concentration following administration. Reference to plasma half-life includes plasma half-life during the α-, β-, and/or γ-phase. Plasma half-life can be assessed using methods well known in the art. For example, assays in which a peptide(s) or therapeutic(s) or combinations thereof is administered to subjects can be performed, and the plasma half-life of the peptide(s) or therapeutic(s) or combinations thereof assessed by measuring the amount of the peptide(s) or therapeutic(s) or combinations thereof in the plasma at various time points. The t½ß, for example, is calculated as −ln2 divided by the negative slope during the terminal phase of the log-linear plot of the plasma concentration-versus-time curve. In some examples, the plasma half-life can aid the skilled artisan in determining when a secondary peptide or therapeutic or combinations thereof can be administered to a subject, such as, for example, in a rotational therapy regimen described herein.

As used herein, clearance refers to the removal of an agent, such as a peptide, from the body of a subject following administration. Clearance can be assessed using methods well known in the art. For example, assessment of peptide levels in blood or serum or another fluid from a patient administered a peptide or combination of peptides or therapeutics can be performed, and the clearance of the peptides from the body assessed by measuring the amount of the peptide(s) in the plasma at various time points and calculating the clearance as Dose/AUC 0-inf In some examples, clearance of a peptide results in a decrease in the circulating peptide levels in serum compared to circulating levels directly or recently after peptide administration. The clearance of administered peptide(s) or therapeutic(s) or combinations thereof can result in a decrease in circulating levels by at least or about 1%, 2%, 3%, 4%, 5%, 6%, 7%, 8%, 9%,0 10%, 20%, 30%, 40%, 50%, 60%, 70%, 80%, 90%, 91%, 92%, 93%, 94%, 95%, 96%, 97%, 98%, or 99%, compared to circulating levels directly or recently after peptide administration.

As used herein, the term assess or assesses and grammatical variations thereof, is intended to include quantitative and qualitative determination in the sense of obtaining an absolute value for the activity of a pharmaceutical(s) or therapeutic(s) or phenotype(s), and also of obtaining an index, ratio, percentage, visual or other value indicative of the level of the activity. Assessment can be direct or indirect. For example, assessing the effect of administration of a combination therapy provided in accord with a rotational regimen described herein on a subject for weight loss can be evaluated by a variety of metrics, including fat, weight, water, and muscle loss or gain. Assessing the effect of administration of a combination therapy provided in accord with a rotational regimen described herein also can be evaluated by measuring levels of the therapeutics or other molecules in the bloodstream. In some examples, a decrease in the amount of the therapeutic(s) such as, for example, a therapeutic peptide or combinations of peptides in the bloodstream or serum can indicate that another peptide(s) or therapeutic(s) or combinations thereof can be administered to the subject. For example, a secondary therapeutic(s) (i.e., peptide e.g., therapeutic peptide) or combinations thereof that is different from the initially administered therapeutic(s)) (i.e., peptide(s) e.g., therapeutic peptide(s)) or combinations thereof can be administered when the concentration of the primary is decreased by at least or about or about at least 1%, 2%, 3%, 4%, 5%, 6%, 7%, 8%, 9%, 10%, 20%, 30%, 40%, 50%, 60%, 70%, 80%, 90%9, 91%, 92%, 93%, 94%, 95%, 96%, 97%, 98%, 99%, or 100% compared to the level or amount of the therapeutic(s) directly after administration, where the level of amount of the therapeutic(s) directly after administration is assessed at least or at least about 1 minute, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 6 hours, 12 hours or more after administration.

As used herein, the term “subject” refers to an animal, including a mammal, such as a human being. Subjects include human patients.

As used herein, a “patient” refers to a human subject. In some examples “patients” or “subjects” are humans who participated in a therapeutic regimen, such as a therapeutic regimen described in the examples herein. In some examples a “patient” is a human who has a chronic condition, disorder, or disease, such as overweight or obesity. Patients or subjects can be treated with the rotational combinatorial therapy herein.

As used herein, “animal” includes any animal, such as, but not limited to, primates including humans, gorillas, and monkeys; rodents, such as mice and rats; fowl, such as chickens; ruminants, such as goats, cows, deer, and sheep; pigs; and other animals. Non-human animals exclude humans as the contemplated animal.

As used herein, a “control” refers to a sample that is substantially identical to the test sample, except that it is not treated with a test parameter, or, if it is a plasma sample, it can be from a normal volunteer not affected with the condition of interest. A control also can be a subject, such as a subject that is not treated with rotational combinatorial therapy provided herein, or is treated with a monotherapy, or combination therapy that is not rotated, or a placebo. A control also can be an internal control.

As used herein, a “composition” refers to any mixture of two or more products or compounds, for example, but not limited to, peptides, therapeutic molecules, agents, modulators, and regulators. A composition can be, for example, a solution, a suspension, an emulsion, a liquid, a powder, a paste, aqueous or non-aqueous formulations, and any combination thereof.

As used herein, an “article of manufacture” is a product that is made and sold. As used throughout this application, the term is intended to encompass peptides or drugs, or therapeutics and combinations thereof contained in articles of packaging.

As used herein, “fluid” refers to any composition that can flow. Fluids thus encompass compositions that are in the form of semi-solids, pastes, solutions, aqueous mixtures, gels, lotions, creams and other such compositions.

As used herein, a “kit” refers to a packaged combination, optionally including reagents and other products and/or components for practicing methods using the elements of the combination. For example, kits containing a combination of pharmaceuticals provided herein and another item for a purpose including, but not limited to, administration, diagnosis, and assessment of a biological activity or property are provided. Kits optionally include instructions for use.

As used herein, the singular forms “a,” “an” and “the” include plural referents unless the context clearly dictates otherwise. Thus, for example, reference to a compound, comprising “a therapeutic” includes combinations with one or a plurality of therapeutics.

As used herein, ranges and amounts can be expressed as “about” a particular value or range. About also includes the exact amount. Hence, “about 5 hours” means “about 5 hours” and also “5 hours.”

As used herein, “optional” or “optionally” means that the subsequently described event or circumstance does or does not occur, and that the description includes instances where said event or circumstance occurs and instances where it does not. For example, an optionally included therapeutic means that the therapeutic is included or is not included.

### B. OVERVIEW

Provided herein are combinatorial treatment protocols and rotational combinatorial treatment protocols in which diseases, disorders, and conditions are treated by targeting a plurality of molecules and/or pathways involved in the disease, disorder, or condition, generally to reduce or avoid desensitization to a particular treatment, and also can reduce toxicity and adverse side effects. The combination of treatments can be rotated so that a subject is treated with different combinations of treatments for limited periods of time. Also provided are methods for developing such protocols, including selecting a disease, disorder, or condition for treatment with a combinatorial protocol and/or a rotational combinatorial protocol. These protocols and methods are exemplified herein with respect to obesity, and several other diseases, disorders, and conditions that are difficult to treat and that become resistant to treatment over time.

Obesity is exemplar of a disease, disorder, or condition, as shown herein that can be treated with a combinatorial, particularly a rotational combinatorial treatment protocol. Obesity is a medical condition in which excess body fat has accumulated. The excess body fat has adverse effects on health (see, e.g., “Obesity and overweight Fact sheet No 311,” published by the World Health Organization (WHO) in January 2015. Retrieved 2 Feb. 2016). Obesity increases the likelihood of various diseases and physical and mental conditions. For example, these increases are manifested in metabolic syndrome (Haslam et al., (2005) Lancet 366 (9492): 1197-209), and include cardiovascular diseases, high blood pressure, high blood cholesterol, high triglyceride levels, diabetes mellitus type 2, obstructive sleep apnea, certain types of cancer, osteoarthritis, and depression (Haslam et al., (2005) Lancet 366 (9492): 1197-209). Obesity reduces life expectancy. (Jura et al., (2016) Age (Dordr) 38(1):23; Peeters et al., Ann Intern Med (2003) 138(1):24-32).

These health complications either are directly caused by obesity or indirectly related to obesity through mechanisms sharing a common cause such as a poor diet, a sedentary lifestyle, and/or genetics. The strength of the link between obesity and specific conditions varies; one of the strongest is the link with type 2 diabetes; approximately 64% and 77% of cases of diabetes in men and women, respectively, can be attributed to excess body fat (Maggio et al., Endocrinol Metab Cin North Am. (2003) 32(4):805-22).

The biochemical link between the development of obesity and health consequences fall into two broad categories: those attributable to the effects of increased fat mass, such as osteoarthritis, obstructive sleep apnea, and social stigmatization, and those due to the increased number of fat cells, such as diabetes, cancer, cardiovascular disease, non-alcoholic fatty liver disease (Stenkula et al., Am J Physiol Regul Integr Comp Physiol. (2018) 315(2):R284-R295). Increases in body fat alter the body's response to insulin, potentially leading to insulin resistance. Increased fat also creates a proinflammatory state and a prothrombotic state (Kawai et al., Am J Physiol Cell Physiol. (2021) 320(3):C375-C391; Moghbeli et al., Adv Clin Chem (2021) 101:135-168; Samad et al., Blood (2013) 122(20):3415-22; Bovolini et al., Int J Sports Med. (2021) 42(3):199-214).

Provided herein are combinatorial protocols for treating obesity. Also provided are rotational combinatorial protocols for treating a variety of diseases, disorders, and conditions. Combinatorial protocols involve the administration of a plurality of treatments, each of which generally targets different pathways involved in the disease, disorder, or condition. The combinatorial protocols generally involve combination of at least three different treatments. The combinatorial protocol(s) can be integrated into a rotational combinatorial protocol in which different combinations of treatment are rotated. Rotational protocols can be selected where monotherapy or combination of treatments become less effective or require higher doses to be effective. Provided herein are exemplary rotational combinatorial protocols and methods of developing such protocols.

### C. COMBINATORIAL THERAPY AND ROTATIONAL COMBINATORIAL THERAPY

Individuals treated with therapeutic agents for conditions, disorders, or diseases, such as chronic conditions, disorders, and diseases, are at risk of developing a tolerance or resistance to the therapeutic effects of the medications. In the context of biological systems and pharmacology, the activation of cell surface receptors can trigger regulatory processes that restrict signaling duration and/or strength. Downregulation is a process by which a particular cellular component, such as a protein (e.g., receptor), decreases in abundance or decreases activity responsive to an external stimulus (i.e., long term administration of an agonist). For example, the expression of a specific receptor or decrease in total receptor number in the cell can decrease (i.e., by enhanced receptor degradation or decreased receptor synthesis or ligand-induced internalization of receptors (endocytic downregulation)). In response to increased or long-term exposure to an agonist, such as a hormone or neurotransmitter or other signaling molecule, the decrease in receptor activation or number can reduce cell sensitivity to the agonist. In the presence of sustained ligand stimuli, the receptor system enters a refractory state thereby preventing the cell from over-responding to the ligand.

This negative feedback mechanism is important in drug treatment, where chronic exposure to a drug and/or therapeutic molecule can become less effective upon long-term or chronic exposure. An example of this negative feedback mechanism are hormone receptors feedback systems. When receptors have been chronically exposed to an excessive amount of a ligand or exposed to a ligand for a prolonged period of time, either from endogenous mediators or from exogenous drugs, this can result in ligand-induced desensitization or internalization of that receptor. Downregulation, therefore, effectively minimizes overstimulation of a pathway to prevent disruption of an organism's internal processes as a result of the increased or chronic activation.

Downregulation is a cellular mechanism characteristic of long-term administration of therapeutic agents. Understanding downregulation and cellular responses to chronic drug exposure is fundamental to developing an effective drug regimen for treating a complex disease state. Targeted therapeutics that are rotated to decrease receptor or cellular downregulation, or other cellular responses that decrease efficacy or activity of a therapeutic agent can be used to treat diseases, disorders, and conditions, such as obesity, and have wide reaching implications across medical specialties for drug development for chronic conditions in which there are a plurality of targets for therapeutic intervention for in which desensitization to treatments occurs.

Combinatorial treatments involve the use of two or more, generally at least three, different treatments that target different pathways for treating a disease, disorder, or condition. The combinatorial treatments, particularly if they start to lose effectiveness for treatment of chronic diseases, disorders, and conditions, can be rotated, which avoids the problems, particularly desensitization, which occurs with long-term therapies for diseases, disorders, and conditions, such as obesity, Parkinson's disease, and other chronic diseases, disorders, and conditions that require treatment for many months, and generally for life.

Rotational combinatorial therapy involves protocols/regimens for treatment of a disease, disorder, and/or condition, in which two or more different therapies (or therapeutics) in combination are administered such that the combinations of the therapies are rotated for predetermined or multiple rounds of treatment. Rotational combinatorial therapy or pharmacology is a protocol for treating a disease, disorder, and/or condition, in which a number of therapeutics or treatments, at least two, different therapeutics/treatments for a disease are administered according to a schedule in which different therapeutics and combinations thereof are administered. Each therapeutic/treatment can be different, having a different target or mode of action. There also can be overlap of the therapeutics in the combinations, especially if there is a time period (gap) between therapies (i.e., giving the pathway time to ‘recover’ or regain the ability to become activated). As set forth below, a combination in the rotational protocol can contain more than one therapeutic, wherein each targets the same pathway to effect a therapeutic effect, which can, in some instances, achieve a synergistic effect such that lower doses, compared to monotherapy, of each therapeutic can be administered. In these examples the timing for rotation may be decreased. Because the medications are used in a therapeutic combination, in some examples a medication or medications in the combination can be used at lower dosages than treatment with a monotherapy, to reduce the risk or severity of adverse side effects.

In accord with rotational combinatorial protocols, each combination of treatments is administered for a predetermined time, generally at least a week, and then replaced by a different combination, which is administered for a predetermined period of time. Each combination can include a drug in common, and/or one of the rotations can include only a single therapeutic, but each combination is different from the other combinations. A rotational protocol can include at least two different combinations that are rotated, and generally includes at least three combinations of therapeutics and treatments that are rotated. The protocol can be repeated a plurality of times.

The predetermined rounds for treatment and timing for treatment can be modified if the subject demonstrates signs or signals that a pathway is downregulated, or the subject becomes less responsive to the therapeutic effects of the therapy or the subject exhibits an increase in adverse side effects. For example, the combinations can be switched prior to the predetermined time. In other examples, if the subject exhibits a decrease in the side effects of the medication, this can indicate that downregulation of the pathway has occurred or is occurring, and the dosage can be increased or another therapeutic that activates a different pathway can be substituted. For example, if administration of a medication results in a side effect of jitteriness, and after a set period (i.e., 2 months) the jitteriness diminishes, the diminution can indicate that the therapeutic pathway is downregulated and medication that acts through a different pathway can be substituted.

The regimen can involve a first round in which one, two, or more of the therapeutics/treatments are administered for a period of time, followed by second round in which a different therapeutic/treatment or different combination of therapeutics is/are administered for another period of time, and repeating round one and round two, or administering a third round of pharmaceuticals for a predetermined period of time.

Depending on the number of therapeutics that are part of the treatment, as well as the disease, disorder, or condition, and particulars of the treated subject, different combinations of therapeutics are administered for each time period, providing different combinations of therapeutics rotated for predetermined periods of time. In some examples, the time periods for treatment are not predetermined; the different combinations are administered for each period of time, and the combinations of therapeutics are rotated when the patient is showing reduced therapeutic benefit(s), or the patient is showing increased or intolerable side effects from the therapeutics. The therapy involves at least two different therapeutics, administered sequentially, and then together, or administered together, and typically involves at least three different therapeutics, usually drugs, administered separately or in combinations. Generally, all combinations and orders of administration can be included in the protocol. Hence, the name rotational combinatorial pharmacology.

A regimen of a rotational combinatorial therapy described herein comprises two or more different therapeutic combinations (also referred to as clusters), where each combination includes two or more medications or treatments. Each combination is rotated for a predetermined time of treatment. Rotation can revert to combination one or any other combination in the regimen.

In some examples, a rotational combinatorial treatment or therapy includes the administration of a first therapeutic agent or combination of therapeutic agents and, after a set time point and in fixed intervals, administration of a different therapeutic agent or combination (cluster) of therapeutic agents, where the second combination (cluster) of agent(s) target a receptor/and or pathway distinct from at least one of the initial therapeutic agent(s), and, optionally, any subsequent combinations are administered after a set time point and in fixed intervals. In exemplary regimens described herein, the first therapeutic agent(s) combination is/are administered and then, such as three months later, a second therapeutic agent(s) combination is/are administered. In examples, there is a third agent(s) combination administered after the second agent(s), at a set time point and after the fixed interval. In some examples, there are multiple rounds of administration of agent(s) at fixed time points after the fixed interval(s). Treatment can proceed for years, and can, if necessary be administered for life. Any of the therapeutic agents used in the rotational therapy herein can be a pharmaceutical or non-pharmaceutical therapy.

In some examples, the combinations can be administered in accord with any of the regimens set forth in the any of following tables:

TABLE 1
Exemplary Regimen for a Rotational Combinatorial
Therapy Rotated at three-month intervals

| Time Period | Combination |
|---|---|
| Months 1-3 | A |
| Months 4-6 | B |
| Months 7-9 | C |
| Months 10-12 | A |
| Months 13-15 | B |
| Months 16-18 | C |
| Months 19-21 | A |
| Months 22-24 | B |
| Months 25-27 | C |

Further rotated every three months A, B, C as long as needed (i.e., as long as determined necessary by the skilled physician)
Where A, B, and C are each a combination of a plurality of therapeutics, where the combination of therapeutics in each of A, B, and C is not the same; combinations can include the same therapeutic, but the combinations of therapeutics are different

TABLE 2
Exemplary Regimen for a Rotational Combinatorial
Therapy Rotated at three-month intervals

| Time Period | Combination |
|---|---|
| Months 1-3 | A |
| Months 4-6 | B |
| Months 7-9 | C |
| Months 10-12 | D |
| Months 13-15 | B |
| Months 16-18 | C |
| Months 19-21 | A |
| Months 22-24 | B |
| Months 25-27 | C |

Rotated every three months, as above, as long as needed (i.e., as long as determined necessary by the skilled physician)
Where A, B, C, and Dare each a combination of a plurality of therapeutics, where the combination of therapeutics in each of A, B, C and D is not the same

TABLE 3
Exemplary Regimen for a Rotational Combinatorial
Therapy Rotated at One Month Intervals

| Time Period | Combination |
|---|---|
| Month 1 | A |
| Month 2 | B |
| Month 3 | C |
| Month 4 | A |
| Month 5 | B |
| Month 6 | C |
| Month 7 | A |
| Month 8 | B |
| Month 9 | C |

Rotated every one month, as above, as long as needed (i.e., as long as determined necessary by the skilled physician)
Where A, B, and C are each a combination of a plurality of therapeutics, where the combination of therapeutics in each of A, B, and C is not the same

Tables 4 and 5, below, set forth rotational combinatorial therapy regimens that are rotated at 4-month and 6-month intervals, respectively. In some examples herein, downregulation generally occurs after at least 3 months and, thus, rotations of combinations generally occur at or about 3 months or later. In some examples, such as where downregulation of particular pathways is slow or takes longer than 3 months, regimens such as those set forth in Tables 4 and 5 are employed.

TABLE 4
Exemplary Regimen for a Rotational Combinatorial
Therapy Rotated at Four Month Intervals

| Time Period | Combination |
|---|---|
| Months 1-4 | A |
| Months 5-8 | B |
| Months 9-12 | C |
| Months 13-16 | D |
| Months 17-20 | B |

Rotated every four months, as above, as long as needed (i.e., as long as determined necessary by the skilled physician)
Where A, B, C, and D are each a combination of a plurality of therapeutics, where the combination of therapeutics in each of A, B, C and D are not the same

TABLE 5
Exemplary Regimen for a Rotational Combinatorial
Therapy Rotated at Six Month Intervals

| Time Period | Combination |
|---|---|
| Months 1-6 | A |
| Months 7-12 | B |
| Months 13-18 | C |
| Months 19-24 | D |
| Months 13-15 | B |

Rotated every six months, as above, as long as needed (i.e., as long as determined necessary by the skilled physician)
Where A, B, C, and D are each a combination of a plurality of therapeutics, where the combination of therapeutics in each of A, B, C and D are not the same

Regimens for rotating and administering therapeutic combinations can be developed in accord with the description provided herein, and by modifying regimens described herein and those set forth in the following tables:

TABLE 6.1
Exemplary Regimen for a Rotational Combinatorial
Therapy Rotated at three-month intervals

| Timing of | Therapeutic | |
|---|---|---|
| Administration | Combination | Function |
| Months 1-3 | | |
| AM | Therapeutic #1 | Appetite suppression |
| | Therapeutic #2 | Increase Insulin sensitivity |
| | Therapeutic #3 | Accelerate Glycogenolysis and Lipolysis |
| | Therapeutic #4 | Decrease food intake, body weight |
| PM | Therapeutic #5 | Muscle enhancement |
| | Therapeutic #11 | Inhibit Gastric Emptying |
| Months 4-6 | | |
| AM | Therapeutic #6 | Inhibit gastric motility |
| | Therapeutic #2 | Increase Insulin sensitivity |
| | Therapeutic #3 | Accelerate Glycogenolysis and Lipolysis |
| | Therapeutic #7 | Reduce eating, gastric acid secretion, limit |
| | | the rate of gastric emptying |
| PM | Therapeutic #8 | Muscle enhancement |
| | Therapeutic #12 | Increase glycogenolysis |
| Months 7-9 | | |
| AM | Therapeutic #2 | Increase Insulin sensitivity |
| | Therapeutic #3 | Accelerate Glycogenolysis and Lipolysis |
| | Therapeutic #10 | Enhance the body weight-lowering and |
| | | glucose-lowering efficacy of GLP-1 |
| PM | Therapeutic #5 | Muscle enhancement |
| | Therapeutic #13 | Accelerate Glycogenolysis and Lipolysis |

TABLE 6.2
Exemplary Regimen for a Rotational Combinatorial
Therapy Rotated at three-month intervals

| Timing of | Therapeutic | |
|---|---|---|
| Administration | Combination | Function |
| Months 1-3 | | |
| AM | Therapeutic #6 | Inhibit gastric motility |
| | Therapeutic #2 | Increase Insulin sensitivity |
| | Therapeutic #3 | Accelerate Glycogenolysis and Lipolysis |
| | Therapeutic #7 | Reduce eating, gastric acid secretion, limit the |
| | | rate of gastric emptying |
| PM | Therapeutic #5 | Muscle enhancement |
| | Therapeutic #11 | Inhibit Gastric Emptying |
| Timing of | Therapeutic | Function |
| Administration | Combination | |
| Months 4-6 | | |
| AM | Therapeutic #1 | Appetite suppression |
| | Therapeutic #2 | Increase Insulin sensitivity |
| | Therapeutic #3 | Accelerate Glycogenolysis and Lipolysis |
| | Therapeutic #4 | Decrease food intake, body weight |
| PM | Therapeutic #8 | Muscle enhancement |
| | Therapeutic #12 | Increase glycogenolysis |
| Months 7-9 | | |
| AM | Therapeutic #2 | Increase Insulin sensitivity |
| | Therapeutic #3 | Accelerate Glycogenolysis and Lipolysis |
| | Therapeutic #10 | Enhance the body weight-lowering and |
| | | glucose-lowering efficacy of GLP-1 |
| PM | Therapeutic #5 | Muscle enhancement |
| | Therapeutic #13 | Accelerate Glycogenolysis and Lipolysis |

TABLE 6.3
Exemplary Regimen for a Rotational Combinatorial
Therapy Rotatec at three-month intervals

| Timing of | Therapeutic | |
|---|---|---|
| Administration | Combination | Function |
| Months 1-3 | | |
| AM | Therapeutic #2 | Increase Insulin sensitivity |
| | Therapeutic #3 | Accelerate Glycogenolysis and Lipolysis |
| | Therapeutic #10 | Enhance the body weight-lowering and |
| | | glucose-lowering efficacy of GLP-1 |
| PM | Therapeutic #5 | Muscle enhancement |
| | Therapeutic #11 | Inhibit Gastric Emptying |
| Months 4-6 | | |
| AM | Therapeutic #1 | Appetite suppression |
| | Therapeutic #2 | Increase Insulin sensitivity |
| | Therapeutic #3 | Accelerate Glycogenolysis and Lipolysis |
| | Therapeutic #4 | Decrease food intake, body weight |
| PM | Therapeutic #8 | Muscle enhancement |
| | Therapeutic #12 | Increase glycogenolysis |
| Months 7-9 | | |
| AM | Therapeutic #6 | Inhibit gastric motility |
| | Therapeutic #2 | Increase Insulin sensitivity |
| | Therapeutic #3 | Accelerate Glycogenolysis and Lipolysis |
| | Therapeutic #7 | Reduce eating, reduces gastric acid secretion, |
| | | limits the rate of gastric emptying |
| PM | Therapeutic #5 | Muscle enhancement |
| | Therapeutic #13 | Accelerate Glycogenolysis and Lipolysis |

### Exemplary of Therapeutics in Tables 6 are the Following:

Therapeutic #1=GLP-1
Therapeutic #2=Adiponectin
Therapeutic #3=Leptin
Therapeutic #4=Oxyntomodulin
Therapeutic #5=Sermorelin
Therapeutic #6=Peptide YY (PYY)
Therapeutic #7=Amylin
Therapeutic #8=tesamorelin
Therapeutic #9=Pancreatic polypeptide (PP)
Therapeutic #10=Enterostatin/GIP
Therapeutic #11=Cholecystokinin (CCK)
Therapeutic #12=Vasoactive Intestinal Therapeutic
Therapeutic #13=Glicentin

TABLE 6.4

| | Examples of Therapeutics that have the |
|---|---|
| Activity | Activity |
| Inhibits gastric emptying | GLP1, Amylin, Pancreatic polypeptide |
| | Therapeutic |
| Enhances satiety | GLP1, Leptin, PYY, Amylin, |
| | Enterostatin/gastric inhibitory peptide (GIP), |
| | Cholecystokinin (CCK), Glicentin |
| Increases insulin release/sensitivity | GLP1, Adiponectin |
| Modulation of energy expenditure | Leptin, oxyntomodulin, Glicentin |
| Muscle enhancement | Sermorelin, Tesamorelin |
| Smooth muscle (intestinal) relaxation | Vasoactive intestinal peptide (VIP) |

The tables above and description herein provide exemplary of regimens for administration of therapeutic combinations, and rotational combinations for weight loss as exemplary of a condition that can be treated by combinatorial and rotational combinatorial methods and regimens provided herein. The combinations, therapeutics in the combinations, dosages, timing and other aspects of the regimens can be modified as required to achieve a therapeutic benefit and/or decrease adverse side effects. The skilled person can select from among medications that have the recited function and combine them to achieve additive and synergistic effects.

The combinatorial treatment or therapy and rotational combinatorial treatment or therapy provides an improved treatment of diseases and disorders compared to treatment with monotherapies, such as disease states that result from derangements of multiple molecular or cellular pathways or a that show a decrease or plateau in the efficacy of the monotherapy; the combinatorial therapy and/or rotational combinatorial therapy described herein modulates the multiple pathways derangements to ameliorate negative effects of the disease or disorder. Modulation of multiple pathways provides improved therapeutic benefits compared to monotherapy or even dual therapy, which target fewer molecular or cellular targets or pathways. Rotating multiple therapeutics (i.e., pharmaceuticals) is an effective method of preventing molecular and cellular adaptation that occurs after continued (i.e., long-term) treatment with monotherapy. Rotating multiple therapeutics or therapies also can prevent cellular adaptation that occurs after therapy with combination therapy (i.e., treatment with more than one therapeutic) that is not rotated. For example, rotating multiple therapeutics or therapies also can prevent cellular adaptation that occurs after therapy with a monotherapy or combination therapy that is administered for a prolonged period (e.g., longer than 3 months).

In some examples the therapeutics in the combination therapy and/or rotational combinatorial therapy are synergistic; one therapeutic (i.e., drug) enhances the clinical activity of another therapeutic (i.e., drug) when used in the combination, and the cumulative effects of the therapeutics exceed the expected clinical benefit of the sum of the multiple drugs in the combination. In other examples, increased efficacy of the combination works through independent drug action rather than a synergistic effect of the therapeutics in combination; the therapeutic benefit is attributable to a single therapeutic in the combination and the benefits over monotherapy are due to increasing the odds that the combination includes a drug that is effective for a particular patient.

Provided herein are combination therapies for use in a rotational regimen for use in the treatment of chronic conditions, such as conditions lasting more than three months, and conditions where patients develop a tolerance to treatment. The rotational combinatorial therapy is for treating chronic conditions with a plurality of known treatments or known pathways associated with disease progression or pathology. A rotational combinatorial therapy regimen can be developed by identifying known treatments/therapies the disease(s), disorder(s), and/or condition(s) for treatment; identifying the pathways, mechanism of actions or targets for treatment; selecting treatment(s) and/or therapies that include treatment(s) and/or therapies that activate different pathways or that have different mechanisms of action; creating combinations that include at least two therapeutics known to activate different molecular and/or cellular pathways and design a regimen for administration of the combinations; and creating a regimen to administer multiple rounds of treatment, with different combinations. In some embodiments, combinations used in one or more rounds of rotation can include a single therapeutic. Generally, all combinations that are rotated include two or more therapeutics or treatments.

### 1. Identification of Diseases, Disorders, or Conditions for Treatment

The combinatorial rotational therapy provided herein can be used in methods of therapy for treating diseases, disorders and/or conditions where the affected patients are at risk of developing a tolerance or resistance to the therapeutic effects of the therapeutic(s) (i.e., medications). The diseases, disorders, and/or conditions have more than one target/pathway for therapeutic intervention. Generally, the disease, disorder, or condition is chronic or requires extended or life-long treatment. Other diseases, disorders and/or conditions are those where the afflicted individuals are at risk of developing a tolerance or resistance to the therapeutic effects of the medications and include chronic conditions, where the disorder or its effects are persistent or long-lasting, or is a disease that develops over time. Patients with a variety of diseases or conditions (e.g., chronic conditions) can benefit from a rotational combinatorial therapy described herein. These conditions include, for example those that can be treated by: 1) a combination of more than one medication and 2) a rotational aspect, such as conditions where the prescribed therapeutics have the potential to decrease efficacy over time. Thus, the methods herein, address the problems of inadequate effectiveness of a monotherapy, and the desensitization. Combinations of a plurality of drugs can improve effectiveness; combining this with rotation of the combinations of the drugs (or of the drugs), leads to sustained weight loss.

A disease, disorder, or condition for treatment with a rotational combinatorial therapy described herein is one where 1) there are a plurality of different therapeutics/treatments known or that can be developed for treating the disease, disorder, or condition, such as where there are a plurality of therapeutic intervention pathways or targets; and/or 2) the disease, disorder, or condition generally is chronic; and/or 3) the disease, disorder, or condition is one for which treatment often fails because tolerance to the therapeutics/treatments develops and/or the therapeutics/treatments become ineffective over time.

a. Disease, Disorder, or Condition with a Plurality of Known Treatments

The rotational combinatorial therapy provided herein can be used for treatment of any condition where there is an effective treatment or a plurality of different known therapeutics and/or treatments. For example, the rotational combinatorial therapy provided herein can be used for treatment of any condition where there is a known pharmaceutical or plurality of pharmaceuticals that ameliorate(s) symptoms of the condition. The rotational combinatorial therapy provided herein also can be used for treatment of any condition with a previously characterized mechanism of action or pathway, such that a therapeutic can be developed for treating the disease or disorder or condition For example, a disease, disorder, or condition in which there are a plurality of different known molecular targets for treatment or multiple known molecular or cellular pathways that are involved in disease progression can be treated with the rotational combinatorial therapy described herein. For example, the rotational combinatorial therapy described herein can be used to target multiple pathways that are associated with a disease or disorder, but where a pharmacotherapy has not yet been developed.

In disorders involving a host of cellular receptor(s), activation of the receptor(s) can lead to activation of an array of cellular pathways. Administration of a monotherapy, which activates or effects activation of a single pathway can have limited efficacy for improving the disorder. An approach for reducing the symptoms or effects of the disorder can require administering a combination of compounds targeting the different pathways and processes.

The combination therapies described herein can include treatments and/or therapies that target multiple pathways, and diseases or conditions for treatment where targeting a single pathway or mechanism will not confer significant or complete amelioration of the disease state or symptoms. For example, the rotational combinatorial therapy provided herein can be used to diseases where administration of a single therapeutic in animal models or in human patients has a limited therapeutic effect.

In some examples, the disease, disorder, and/or condition is a multifactorial process where the therapeutic benefit from a combination of different therapeutics exceeds the therapeutic benefit of a monotherapy. For example, the disease, disorder, or condition is influenced by multiple genes (polygenic) and resultant genetic pathways, and generally in combination with lifestyle and environmental factors, such as exercise, diet, or pollutant exposures. The combination of genetic and environmental factors act together in concert to trigger the development and progression of the multifactorial disease.

The concept of rotational and/or combinatorial pharmacology provides a method for treating complex disease processes, such as obesity, which involve multiple pathways, in which monotherapy and dual therapy have shown limited success. The combinatorial pharmacology for weight loss as exemplified herein is exemplary of a condition that can be treated with a rotational therapy. Other conditions that involve multiple molecular and/or cellular pathways also can be treated by rotational combinatorial pharmacology, such as conditions where monotherapies are inadequate. Combination therapy also can overcome limitations of monotherapies, where multiple medications, by virtue of the number of medications in the combination, increases the chance the subject will be responsive to a particular therapeutic.

b. Chronic Conditions

Chronic conditions require ongoing therapeutic intervention and can negatively impact or limit the daily activities or the quality of life of the affected individual. Chronic conditions include conditions in which known therapeutics are ineffective or that do not significantly or completely ameliorate the symptoms or characteristics of the condition. Chronic conditions also include conditions where patients treated with monotherapies are not responsive to treatment or who have not improved on the monotherapy or who continue to have adverse symptoms of the condition despite the therapeutic intervention. Chronic conditions also include conditions where patients treated with combination therapies (i.e., continuous and/or prolonged administration of more than one therapeutic) are not responsive to treatment or who have not improved on the combination therapies or who continue to have adverse symptoms of the condition despite the therapeutic intervention.

Chronic conditions are conditions that persist over time. In some examples, chronic conditions last more than 3 months, 6 months, 1 year, 2 years, 3 years, 4 years, 5 years or more. The rotational combinatorial therapy provided herein is for treating chronic conditions. In some examples, the rotational combinatorial therapy described herein is for treating a chronic condition, disease, or disorder in which the course of the condition, disease or disorder lasts more than about three months. A chronic condition also can be characterized by the amount of time the therapeutics for treating the condition are administered. For example, chronic conditions can be conditions in which therapeutic(s) for treating the condition is/are administered for an extended period of time (i.e., about 3 months, 6 months, 1 year, 2 years, 3 years, 4 years, 5 years or more).

Patients with chronic conditions can be administered a medication or combination of medications for months or years, during which time they can develop a decreased response to the medication(s), such as decreased response due to signal downregulation or desensitization to the medication, and can suffer from side effects from prolonged use of the medication(s). Subjects with chronic conditions can benefit from treatment with combinations of therapeutics that are rotated, which can increase the likelihood of a prolonged therapeutic response compared to treatment with a monotherapy or combination of therapeutics that are not rotated.

Provided herein are combination therapies for use in a rotational regimen for use in the treatment of chronic conditions. In some examples of chronic conditions, patients are administered therapeutic treatment(s) for extended periods of time and can develop a tolerance to treatments, such that the therapeutics for treating the conditions become or are less effective. For example, provided herein are combination therapies for use in a rotational regimen for use in the treatment of chronic conditions, for use in the treatment of overweight or obesity, and/or for weight loss. The combination therapies for use in a rotational regimen also are for use for treating or ameliorating the symptoms of comorbidities of overweight and/or obesity. Exemplary of such comorbidities include diabetes (e.g., diabetes mellitus type 2), cardiovascular disease (i.e., cardiovascular disease leading to heart attack or stroke), high blood pressure, high blood cholesterol, high triglyceride levels, persisting neurodegenerative disorders, metabolic syndrome, obstructive sleep apnea, depression, non-alcoholic fatty liver disease, and cancer, such as but not limited to, pancreatic cancer, breast cancer, prostate cancer, gastric cancer, colon cancer, ovarian cancer, head and neck cancer and others. The combination therapy and rotational regime can be modified to decrease the amount or severity of adverse side effects associated with administration of the therapeutic(s). For example, the dosage of the therapeutic(s) can be lowered, or the therapeutics or therapeutic combinations can be rotated with greater frequency.

Rotating combinations of therapeutics can expose patients to each of the particular therapeutics for a shorter amount of time or to a lower dosage, which can each decrease the chance of developing side effects or the severity of side effects from the particular medication. The rotational combinatorial therapy provided herein can be used to minimize side effects during treatment or therapy for diseases or disorders, compared to treatment with a monotherapy or dual therapy or combination therapy with two or more therapeutics. In some examples, the combination therapy described herein can be used and/or administered at a lower dosage than the dosage of the therapeutic used for monotherapy. For example, the combination therapy is used at a lower dosage than the monotherapy which results in fewer or less severe adverse side effects during treatment.

Patients with a chronic condition who previously were treated with a monotherapy, where the monotherapy did not decrease disease signs or symptoms or where the monotherapy did not inhibit or stop disease progression, can be administered a combination therapy or rotational combinatorial therapy provided herein to improve the therapeutic response or to decrease the adverse symptoms associated with the disease state. In other examples, patients with a chronic condition who were previously treated with a combination therapy, where the combination therapy did not decrease disease signs or symptoms or where the combination therapy did not inhibit or stop disease progression, can be administered a rotational combinatorial therapy provided herein.

A rotational combinatorial therapy also can be used for treating subjects with chronic conditions who have previously been treated with a monotherapy or a combination therapy and who are responsive to the treatment, but where the treatment did not completely ameliorate symptoms of the condition or did not cure the disease. In these examples, a patient can benefit from a combination therapy targeting multiple molecular and/or cellular to target pathways to which the subject will be more responsive. A combination of therapeutics administered in a rotation or regimen described herein can show improved amelioration of symptoms of a chronic condition compared to a monotherapy or combination of therapeutics that are not rotated by decreasing downregulation of therapeutic pathways and activating molecular pathways that were not previously activated (i.e., not previously subject to downregulation).

c. Conditions where Patients Develop a Tolerance to Treatments

Patients with chronic conditions can develop a decreased response (tolerance) to the medication(s) prescribed for treating the condition or for ameliorating symptoms of the condition. Such decreased therapeutic response over time can be related or due to signal downregulation or desensitization to the medication(s). Patients that develop a tolerance to a monotherapy or combination therapy can benefit from a rotational combinatorial therapy to overcome the desensitization, such as desensitization due to receptor downregulation.

A rotational combinatorial therapy, thus, can be used for treating conditions where patients develop a tolerance to treatments. For example, subjects with chronic conditions who have previously been treated with a monotherapy or a combination therapy who are responsive to the treatment (i.e., showed an amelioration of symptoms) for a limited time period but where the treatment was less effective or the subject failed to respond to the treatment after a time period can be treated with a combinatorial rotational therapy. For example, a subject who initially showed amelioration of symptoms of the disease or condition, but where the symptoms increased after prolonged treatment, such as, for example, a subject who showed amelioration of symptoms but where symptoms increased after at least 1 week, 2 weeks, 3 weeks, 4 weeks, 5 weeks, 6 weeks, 2 months, 3 months, 4 months, 5 months, 6 months or more of treatment.

### 2. Development of a Combinatorial Rotational Therapy Regimen

Rotational combinatorial therapies provided herein are for treating a disease, disorder, and/or conditions where there are a plurality of different therapeutics/treatments known or that can be developed for treating the disease, disorder, or condition, and the disease, disorder, or condition generally is chronic and/or is one for which treatment fails because tolerance to the therapeutics/treatments develops or the therapeutics/treatments become ineffective over time so that ultimately treatment of the disease, disorder, or condition fails or becomes increasingly ineffective.

In examples, development of a rotational combinatorial therapy can include a protocol including:
1) identify known treatments/therapies for each disease state or pathways associated with each disease state;
2) identify the pathways, mechanism of actions or targets;
3) select treatments/therapies that activate different pathways, have different mechanism of actions or targets, and/or that are compatible with a rotational therapy;
4) create combinations that include at least 2 therapeutics known to activate different molecular and/or cellular pathways and design a regimen for administration of the combinations; and
5) create a regimen to administer multiple rounds of treatment, with combinations of therapeutics.

Each of these aspects is discussed in turn as follows.

1) Identify Known Treatments/Therapies for Each Disease State or Pathways Associated with Each Disease State

A protocol for developing a rotational combination therapy can include identification of treatments or therapies that are known and/or have previously been characterized for treating a disease, disorder, or condition. For example, a protocol for developing a rotational combination therapy described herein can include identifying treatments or therapeutics that have previously demonstrated activity or efficacy for treating the disease, disorder, or condition or for treating or ameliorating secondary effects or symptoms associated with the disease, disorder, or condition. For example, a therapy or therapeutic is a drug that is approved by the Food and Drug Administration (FDA) for human use in the United States. In other examples, the therapeutic can show efficacy for treating the disease, disorder, or condition or treating or ameliorating secondary effects or symptoms associated with the disease, disorder, or condition in animal models.

In other examples, the therapeutic (i.e., pharmaceutical) for inclusion in the rotational combination therapy described herein can be one that was previously identified as a therapeutic that alters (i.e., activates or inhibits) a pathway associated with the disease, disorder, or condition or symptoms thereof in vitro, or in in vivo and/or ex vivo model systems. The in vitro results or results from model systems can indicate that targeting a particular pathway or pathways or use of a particular therapeutic (i.e., drug/pharmaceutical) or treatment will be effective for treating subjects (i.e., human patients) in which the pathway or pathways is/are implicated or use of a particular therapeutic is predicted to be effective. Studies or demonstrations that a therapy or therapies is/are effective for treating or ameliorating a disease, disorder, or condition can be used to select therapeutics for inclusion in the rotational combination therapy described herein.

In other examples, the therapeutic(s) (i.e., pharmaceutical) for inclusion in the rotational combination therapy described herein can be identified in screens for effective therapeutics for treating a disease or disorder. For example, in vitro screens in model systems that replicate disease characteristics can be conducted for new substances showing activity and additionally for assessing effectiveness for treating a disorder. In some examples, the therapeutic(s) (i.e., pharmaceutical) for inclusion in the rotational combination therapy described herein are identified in vitro in screens conducted in cell culture, in a Boyden chamber, in three-dimensional cultures, in microfluidic systems, using 3D bioprinting, or in other systems that can be used to identify therapeutics that can be for treating a disease, disorder, or condition with the rotational combinatorial therapy described herein.

In examples of a rotational combinatorial therapy for treating obesity or overweight or for effecting weight loss, known weight loss medications, such as weight loss medications described herein can be included in the combination. In some examples, a previously identified weight loss medication can be included, including one or more of mitochondrial uncouplers, amphetamines, thyroid hormones, drug cocktails, neuromodulators, lipase inhibitors, cannabinoid receptor antagonists, gastrointestinal-derived peptides chemically optimized for pharmaceutical use, and others. In some examples, one or more FDA approved medications can be included in the combination therapy. For example, one or more of orlistat, phentermine-topiramate, naltrexone-bupropion, liraglutide, tirzepatide (is a GIP analog that acts on GIP and GLP-1 receptors; sold under the trademark Mounjaro™) dual agonist, and semaglutide can be included in the combinations described herein for treating obesity or overweight or for effecting weight loss. In other examples, FDA approved medications that are for use for a disease or disorder where the subjects exhibit weight loss as a result of the therapeutic regimen, but for which weight loss is not the primary objective, can be included in the combination therapy provided herein, for effecting weight loss.

### 2) Identify the Pathways, Mechanism of Actions or Targets

A protocol for developing a rotational combinatorial therapy can also include identification of pathways, such as, for example, molecular or cellular pathways, associated with the disease, disorder, or condition. The molecular and/or cellular pathways can be used to determine the therapeutic(s) for inclusion in the combinatorial therapy.

Treatments or therapeutics for inclusion in the rotational combinatorial therapy described herein can be selected from compounds that target a pathway associated with the disease, disorder, or condition; or symptoms, or secondary or side effects of symptoms associated with the disease, disorder, or condition. Treatments or therapeutics can activate a pathway associated with the disease, disorder, or condition; or symptoms, or secondary or side effects of symptoms associated with the disease, disorder, or condition, such that activation of the pathway ameliorates symptoms of the disease, disorder, or condition or secondary side effects. Alternately, treatments or therapeutics can inhibit a pathway associated with the disease, disorder, or condition; or symptoms, or secondary or side effects of symptoms associated with the disease, disorder, or condition, such that inhibition of the pathway (i.e., inhibition of an overactive pathway or an off-target pathway) ameliorates symptoms of the disease, disorder, or condition or secondary side effects.

When identifying a pathway for targeting in the rotational combinatorial therapy described herein, biomarkers can signal an abnormal process or a condition of the disease. Biomarkers can be used to identify targets for treatment and potential responses to therapeutics and therapeutic combinations. For example, biomarkers can be used to predict a patient's response to individual therapeutics (i.e., drugs) in a combination. For example, higher receptor expression in a patient can correspond to a better response to a corresponding therapy, and lower expression of receptor activators can indicate a higher or lower chance of responsiveness to a particular therapeutic(s) (i.e., drugs). Thus, biomarker identification and characterization, and use of validated biomarkers (i.e., biomarkers of a particular disease state or status) can help predict patient response and therapy-related side effects and inform selection of therapeutics for inclusion in the combination and/or rotational combinatorial therapy herein.

The skilled artisan can evaluate the literature, for example articles published in the scientific literature, such as, for example, in the US National Library of Medicine and the National Center for Biotechnology Information, which assembles biomedical literature from MEDLINE, life science journals, and online books. The scientific literature can identify and assess molecular and cellular pathways for treating diseases, disorders, and/or conditions described herein. Molecular targets for treating a disease, disorder, or condition can be identified and/or evaluated in the scientific literature and used to determine molecular targets for therapeutics for treating the conditions identified and described herein.

In other examples, a pathway associated with the disease, disorder, or condition or symptoms thereof for treatment with a combination or rotational combinatorial therapy described herein can be identified using in vitro, in vivo, and/or ex vivo model systems. The in vitro results or results from model systems can indicate that targeting a particular pathway or pathways or use of a particular therapeutic (i.e., drug/pharmaceutical) or treatment will be effective for treating subjects (i.e., human patients) in which the pathway or pathways is implicated or use of a particular therapeutic is predicted to be effective. Studies or demonstrations that a molecular and/or cellular pathway plays a role in pathogenesis or progression of a disease or condition can be used to determine therapeutics for including in the combination and rotational combination therapy described herein. The therapeutic (i.e., pharmaceutical) for inclusion in the rotational combination therapy described herein can be one that alters (i.e., activate or inhibit) a pathway associated with the disease, disorder, or condition or symptoms thereof in vitro, or in in vivo and/or ex vivo model systems.

In examples of a rotational combinatorial therapy for treating obesity or overweight or for effecting weight loss, molecular and/or cellular pathways that are altered in gastric bypass patients can be targeted in the therapy by combining therapeutics that target each pathway. For example, molecular and/or cellular pathways that are altered following gastric bypass or other surgical weight loss procedures can be used to formulate a pharmacotherapy for inclusion in the combination and/or rotational combination therapy described herein. For example, neuro-hormonal gut peptides that are altered following bariatric surgery; or exogenous (i.e. rationally designed) peptides that mimic gut peptides or that are gut peptide receptor agonists; or therapeutics that alter expression and/or activation and/or activity of neuro-hormonal gut peptides that are altered following bariatric surgery, such as, for example, therapeutics that increase expression and/or activation and/or activity of neuro-hormonal gut peptides that are increased following bariatric surgery, can be included in the combination therapy described herein. The combinatorial regimens and the rotational combinatorial regimens can mimic the effects of bariatric surgery, particularly gastric bypass surgery, such as the Roux-en-y surgery. Gastric bypass surgery has effects that include decreased absorption of food, including intestinal absorption, on glucose homeostasis, and results in hormonal changes that alter appetite/satiety and energy consumption and other physiological processes. Rerouting food through the gastrointestinal tract leads to changes in gut hormone secretion. Changes in gut hormone levels after RYGB, include increased anorectic hormones, such as GLP-1 and PYY, which induce satiety, and decreased levels of orexigens, such as ghrelin, an appetite-stimulating hormone. The rotational combinatorial methods herein allow a variety of combinations of drugs/treatment to reduce desensitization, and adverse effects, while providing the advantages of gastric bypass surgery by combining administration of various drugs that reduce appetite, and hormones that alter appetite. Combinations of such drugs and treatments are detailed herein and are exemplified in the working examples and throughout the description.

Provided are combinatorial methods and combinatorial rotational methods in which combinations of medications, such as peptide hormones or agonists thereof and/or antagonists of peptide hormones that are reduced following bariatric surgery, are administered. Particular combinations are described herein and exemplified in the working examples.

In other examples of a rotational combinatorial therapy for treating obesity or overweight or for effecting weight loss, molecular and/or cellular pathways associated with satiation, metabolism, hunger and weight gain or loss can be identified and used to formulate targets and or therapeutics for the combination therapy.

3) Select Treatments/Therapies that Activate Different Pathways, have Different Mechanism of Actions or Targets, and/or that are Compatible with a Rotational Therapy

Combination therapies containing at least two, and generally at least three, combinations of a plurality of therapeutics and rotated to prevent desensitization of activated pathways exhibit efficacy that is greater than a monotherapy or combination therapy that is not rotated. The combinations contain therapeutics that activate or are known to activate different molecular or cellular pathways. For example, the combinations contain therapeutics that activate or are known to activate a total of at least two, three, four, five or more different molecular or cellular pathways. By virtue of rotating the therapeutic combinations at set intervals, where the combinations contain therapeutics that target different molecular pathways, there is a decreased probability of downregulation of the pathways and, thus, a decreased chance of lower therapeutic effect.

Therapeutics that decrease in efficacy over time can be included in the combinations herein, including, for example, pharmaceutical medications that activate molecular pathways that are downregulated, leading to decreased efficacy of the pharmaceutical. The combinatorial therapy herein includes rotating from one combination comprising a plurality of therapeutics to a next combination comprising a plurality of therapeutics. The rotational aspect overcomes limitations associated with therapeutics that decrease in efficacy over time; the switch to a new combination results in a continuous therapeutic effect that does not wane, or wanes less than treatment with a monotherapy containing one of the therapeutics in the combination.

In examples, therapeutics for inclusion in the combination for the rotational combinatorial therapy herein are those that do not have severe adverse side effects upon ceasing use of the particular therapeutic. For example, therapeutics that do not induce withdrawal symptoms as a result of ceasing or reducing use of the therapeutic (i.e., medication). When selecting medications for inclusion in the combinations, medications that induce withdrawal syndromes and rebound upon discontinuation can be avoided. Therapeutics for inclusion in the rotational combinatorial therapy are those with a low chance of withdrawal or adverse events upon switching to the next combination of medications or those that do not induce adverse events upon switching to the next combination of medications.

In examples, therapeutics for inclusion in the combinatorial therapy are those that are not delayed in producing a therapeutic effect or are not delayed in ameliorating symptoms of the disease or condition to be treated. The therapeutics included in the rotational combinatorial therapy are administered in rotation, for predetermined periods, such as for at least 1 month, 2 months, 3 months or 4 months or 5 months or 6 months or more, and in some examples rotated at least every 3 months. In these examples, the therapeutic(s) takes effect (i.e., has activity) or the therapeutic effect(s) is/are produced or amelioration of symptoms begins near to the time of initial administration, such as within 1 hour, 2 hours, 3 hours, 4 hours, 5 hours, 6 hours, 8 hours, 10 hours, 12 hours, 18 hours, 24 hours, 36 hours, 48 hours, 72 hours, 4 days, 5 days, 6 days, 1 week, 2 weeks, 3 weeks, 4 weeks, 5 weeks, 6 weeks, 2 months, 3 months, 4 months, 5 months, 6 months or more of treatment. For example, the therapeutic(s) takes effect (i.e., has activity) or the therapeutic effect(s) is/are produced or amelioration of symptoms begins within at least 1 week of initial administration. The therapeutic activity can begin within the first half, third, quarter, or tenth of the rotational time period. For example, for a rotational combinatorial therapy where the combinations are rotated at 3-month intervals, the therapeutics in the combinations can show therapeutic activity within 1 month, and more generally within at least 2 weeks or 1 week or less than 1 week, such as two or three days after initial administration.

Medications that are toxic if administered at high dosages can be included in the rotational combinatorial therapy described herein. Therapeutics (i.e., drugs) that show toxicity or adverse side effects when administered in therapeutic amounts can be included in the combination therapy described herein; the therapeutics used in combination with other, complementary, therapeutics can be administered in lower dosages such that when they are administered in combination with other therapeutics, and in a rotational regimen as described herein, they effect amelioration of symptoms or treatment of the disease, disorder, or condition without the adverse effects. Administration of therapeutics in a rotational regimen described herein can reduce drug toxicity, and adverse side effects, while maintaining or improving clinical efficacy. In some examples, a particular medication in the combination is administered at a lower dosage than the medication would be administered in a monotherapy; administration of the medication at the lower dosage decreases drug associated toxicity and adverse side effects.

Previous treatment (i.e., with a monotherapy or combination therapy) can be used to predict a patient's response to a therapeutic or class of therapeutics or to similar therapeutics (i.e., therapeutics that act through the same molecular pathway). Previous treatment (i.e., with a monotherapy or combination therapy) also can be used to predict whether targeting a particular molecular pathway will be effective in that patient. Patients who have shown a positive response to therapeutics targeting one molecular pathway, can be treated with a rotational combinatorial therapy containing therapeutics that target that same pathway. Information from patients who were not previously responsive, or did not show amelioration of symptoms, after treatment with a monotherapy targeting one molecular pathway, can be used to determine the therapeutics for inclusion in a combination therapy herein. For example, if a patient was not previously responsive to a particular pharmaceutical, a plurality of pharmaceuticals targeting the molecular pathway can be included in a combination therapy. In other examples, if a patient was not previously responsive to a particular pharmaceutical, the pharmaceutical can be excluded from the combination therapy. In other examples, if a patient was not previously responsive to a particular pharmaceutical, the pharmaceutical can be included in the combination therapy with other therapeutics predicted to act synergistically with the pharmaceutical.

Treatments that target different pathways associated with satiation, metabolism, hunger and weight gain or loss can be included in a rotational combinatorial therapy herein for treating overweight, obesity or for weight loss, or for treating comorbidities of overweight or obesity, such as diabetes (e.g., diabetes mellitus type 2), cardiovascular disease (i.e., cardiovascular disease leading to heart attack or stroke), high blood pressure, high blood cholesterol, high triglyceride levels, persisting neurodegenerative disorders, metabolic syndrome, obstructive sleep apnea, cancer, depression, and non-alcoholic fatty liver disease. For example, therapeutics that mimic the physiological effects following gastric bypass can include in a rotational combinatorial regimen as provided herein.

4) Identify Combinations that Include at Least 2 Therapeutics Known to Activate Different Molecular and/or Cellular Pathways and Design a Regimen for Administration of the Combinations

The combination can include therapeutics that target the same pathways or that target different pathways. Therapeutics that can provide complementary benefits (i.e., modulate similar or the same molecular pathways to compound the effects), or therapeutics that act through different molecular pathways to effect positive benefits through a variety of pathways can each be included in the rotational combinatorial therapy described herein. The therapeutics in the combinations generally target different pathways. Selecting therapeutics that target different pathways is generally to prevent redundancy in the pathways that the therapeutics target, such as, for example, where redundancy does not increase the therapeutic benefit over the benefit of a single therapeutic, or when redundancy can increase the likelihood or severity of adverse side effects. Selecting therapeutics that target different pathways also overcomes the decreased therapeutic effect that occurs after desensitization of a particular molecular pathway after prolonged treatment. For example, if a therapeutic in a first combination targets a pathway that is desensitized after use, the therapeutic in the second combination targets a second pathway, which is not desensitized, and, thus, not downregulated; a third combination can contain the first therapeutic, if the desensitization no longer exists, or can contain a third therapeutic that targets a third pathway. The rotation of therapeutics that target different pathways overcomes the reduced therapeutic effect after desensitization of a pathway.

The therapeutics for use in the rotational combinatorial therapy described herein can be therapeutics that are complementary in that one effects treatment of secondary effects (i.e., inflammation) and the other effects treatment of the primary cause of the disorder. For example, one therapeutic in the combination can inhibit production of proinflammatory cytokines that are a downstream effect of the primary disorder, such as, cancer and obesity. In other examples, the combination can include therapies that treat or ameliorate the symptoms of concomitant disease states, such as a treatment (i.e., medication) for hypertension included in a combination for treatment of the primary condition of obesity.

Therapeutics that can provide complementary benefits by effecting treatment of different disease phases can be selected for inclusion in the rotational combinatorial therapy described herein. For example, in multi-phasic diseases states, one therapeutic or combination of therapeutics can effect treatment or amelioration of effects of an acute phase of the disease and the other therapeutic(s) can effect treatment or amelioration of effects of a chronic phase or state of the disease.

In some combinations that include therapeutics that target the same pathways, the overlap (redundancy) is, for example, to ensure the efficacy of the combination, to ensure activation or inhibition of a particular pathway. Therapeutics with overlapping targets also can be included in separate combinations in the therapeutic regimen, such as, for example, combinations that are administered at different points in the therapeutic regimen, and generally with a cycle of the regimen separating administration of the redundant therapeutics.

5) Create a Regimen to Administer Multiple Rounds of Treatment, with Combinations of Therapeutics

The combinatorial rotational therapy described herein includes a regimen for administering the plurality of combinations multiple times, such that there are at least two rounds, and generally at least three, four, five or more rounds of treatment, with different combinations. Various factors can influence the timing for administering the plurality of combinations in the rotational combinatorial therapy provided herein including, but not limited to: the particular therapeutics in the combinations; the particular disease to be treated; the predicted and/or known efficacy of the therapeutics or therapies, alone or in combination with other therapeutics and/or therapies; the time for the therapeutics to demonstrate therapeutic activity (e.g., onset of action); the recommended time for therapeutic administration; the maximum recommended time for therapeutic administration; medication dosages; the characteristics of a patient to be treated, including, but not limited to, gender, weight, age, overall health, comorbidities, and other characteristics; and other particulars of the therapeutic(s), patient and disease, disorder, or condition.

In examples where the rotational combinatorial therapy is for treating a rapidly progressing disease, disorder, or condition, the first combination in the rotational combinatorial therapy can include a therapeutic or therapeutics with a high (i.e., the highest) probability of activity and/or efficacy for treating the disease, disorder, or condition. For treating a disease, disorder, or condition that is less rapidly progressing, where an extended time period for administering a second- or third-line combination therapy can be considered, lower dosages or a less aggressive first combination of therapeutics can be administered. The therapeutics for inclusion in the first line therapy (i.e., therapeutics in the first cycle of the rotation) can depend on the disease, disorder, or condition, to be treated, the disease state, state, the rate of progression of the disease, disorder, or condition, the severity of the disease, disease, disorder, or conditions. As detailed above and below, the skilled physician can modify the therapeutics for inclusion in the rotational combinatorial therapy described herein based on the available therapeutics, and, for example, the disease to be treated, the disease state, the rate of disease progression, the severity of the disease, the particular patient to be treated, and other factors. The rotational aspect of the combination therapy provided herein provides for changing of doses and replacement of ineffective or less effective therapeutics with others that have increase efficacy, for example, based on the patient response.

The time for the drug or therapeutic to exhibit therapeutic activity also can be considered when selecting a drug or other therapeutic for treatment in a rotational treatment regimen provided herein. In one example, a drug or therapeutic used in the rotational combination therapy herein should be active prior to rotation and administration of the next therapeutic combination. For example, if a medication takes one week to show therapeutic activity, the combinations will not be rotated at less than one week. For example, if a medication takes one week to show therapeutic activity, the combinations can be rotated after at least one month, two months, three months or more, in order to show therapeutic activity of the initial combination.

As detailed herein, the timing for rotation of the therapeutic combinations can be determined. Various factors can be considered to identify indicators for a switch from one therapy (i.e., treatment or drug or therapeutic or combination thereof) to another, and the timing for a switch. In some examples, the following factors can influence the decision to switch medications: one or more adverse side effects; change in clinical status (e.g., improvement or worsening of the disorder or condition to be treated); poor drug activity despite administration of a therapeutically effective amount of drug; concomitant pharmacotherapy (i.e., drug-drug interactions); need for a different route of administration (e.g., oral vs. injection), such as, for convenience or for improved adherence to the administration regimen or for drug uptake and/or activity; subject responsiveness to the drug (i.e., poor responsiveness), such as because of receptor down-regulation or due to sex or age or race; and others. Thus, the timing for switching combinations will depend on many factors, including the particular therapeutics in each combination. The information provided herein can be used to develop guidelines for a rotation schedule. In examples, combinations are administered in rotation, for predetermined periods, such as for at least 1 month, 2 months, 3 months or 4 months or 5 months or 6 months or 9 months or 1 year or 15 months or 18 months or 2 years or 27 months or 30 months or 33 months or 4 years or more, and in some examples rotated at least every 3 months. As downregulation can occur after at or about 3 months or less, the combinations can be rotated every three months.

After an administration regimen, such as a rotational regimen described herein, has been implemented in a particular patient, the regimen can be modified to improve the therapeutic effects. The patient's clinical status can be used to modify the treatment regimen or schedule or drugs administered. For example, if a patient develops drug sensitivities, the regimen can be modified to switch to a new therapeutic under an expedited schedule. In other examples, the rotational schedule can be extended if previously unrealized benefits of the therapeutic are achieved with a particular medication.

As detailed herein, the dosage of the individual therapeutics in the combinations for rotation can be determined. Various factors can be considered to identify the dosage for each therapeutic (i.e., drug). The examples and disclosure provided herein can be used to develop guidelines for a rotation schedule, and identify the medications for inclusion in the regimen/schedule. The dose of an individual therapeutic can vary depending on the other therapeutics in the combination, and can vary depending on the doses of the therapeutics in the other combinations in the rotation. The regimen can include guidelines for dose adjustment based on the action of the previously administered medications, relative potency, and other facts that can influence efficacy of the therapeutic(s).

The dose range for each of the individual therapeutics in the combinations provided herein can be adjusted by monitoring the subjects (i.e., patients), such as monitoring plasms or monitoring symptoms or monitoring adverse side effects after administration of the combination(s). The dose administered can be such that the subject will maintain a plasma level to effect amelioration of symptoms of the disease, disorder, or condition for which treatment is administered. The dose also can be a dosage that will effect amelioration of of symptoms of the disease, disorder, or condition for which treatment is administered where the subject does not exhibit adverse side effects, or does not exhibit adverse side effects at a severity that will effect cessation of the treatment.

### D. METHODS OF TREATMENT AND USES

The combinatorial and rotational combinatorial therapy provided herein can be used in methods of therapy for treating chronic conditions, such as, for example, overweight and obesity. In other examples, the combinatorial and rotational combinatorial therapy described herein can be used in methods of therapy for treating chronic conditions such as diabetes (e.g., diabetes mellitus type 2), cardiovascular disease (i.e., cardiovascular disease leading to heart attack or stroke), high blood pressure, high blood cholesterol, high triglyceride levels, persisting neurodegenerative disorders, such as Parkinson's disease, metabolic syndrome, obstructive sleep apnea, cancer, osteoarthritis, depression, and non-alcoholic fatty liver disease.

In the methods, a combination therapy is administered in a rotational regimen to a subject having a chronic condition. As described herein, the first combination of therapeutics, in a combination regimen with a second (and third or more) combination(s) far exceeds the effects of a monotherapy or combination therapy that is not rotated, for treating a chronic condition, such as, for example, obesity. For example, the effects can be synergistic. For example, extent and level of weight loss observed following the combination therapy provided herein achieves results that have heretofore not been achieved with existing non-surgical weight loss therapies, including increased fat loss and muscle gain that surpasses existing treatment regimens.

### 1. Therapeutic Uses of the Combinatorial Therapy

Combinatorial and pulsed-dose pharmacology have been used for a variety of diseases, disorders, and conditions, such as pain control, birth control and treatment of cancer, COPD, Parkinson's disease, Alzheimer's disease, and bacterial infections. These previous uses do not contemplate administering a combination of at least two, generally, at least three, different therapeutics/treatments (i.e., therapeutics having a different target or different mode of action) according to a schedule in which different therapeutics are rotated, and/or the combinations selected are not selected to target different pathways.

Treatment strategies to deal with complex medical issues such as cancer, pain control and pulmonary issues also are described herein to integrate more than one medication (combinatorial pharmacology) administered in a rotational regimen, to improve clinical outcomes. The methods and regimens provided herein are designed to target different pathways involved in disease, disorder, or condition, and to rotate combinations of treatments.

In accord with methods herein, rotational combinatorial therapy for treating complex medical disorders include first identifying known treatments/therapies for each disease state or pathways, and mechanisms of action or targets associated with the disease. Then, treatments/therapies that activate different pathways, have different mechanism of actions or targets, and/or that are compatible with a rotational therapy can be identified. Using previous treatments and pathways/mechanisms/targets associated with the disease state, combinations that include at least 2 therapeutics known to activate different molecular and/or cellular pathways can be selected and a regimen to administer multiple rounds of treatment, with combinations of therapeutics, can be designed. As set forth herein, the methods for selecting therapeutics for inclusion in a rotational combinatorial regimen, and the timing for administration of the therapeutic combinations can be prepared. In examples, the combinations can be administered in rotational regimens for treating, for example, any of the diseases, disorders and/or conditions set forth below.

a. Combination Therapies in Cancer

Several cancer therapeutics are used in combination but without a rotational component to treat tumors in patients. For example, ABVD (Adriamycin, Bleomycin Vinblastine Dacarbazine) can be used as the initial chemotherapy treatment for newly diagnosed Hodgkin lymphoma. ABVD has been the most effective and least toxic chemotherapy regimen available for treating early-stage Hodgkin Lymphoma. One cycle of ABVD chemotherapy is typically given over 4 weeks in two doses, where the first dose of the drug combination is administered on day 1 and the second dose is administered on day 15, where all four of the chemotherapy drugs (Adriamycin, Bleomycin Vinblastine Dacarbazine) are administered intravenously.

Another combination therapy for cancer treatment is known by the acronym CHOP, which includes a combination of cyclophosphamide, doxorubicin hydrochloride (hydroxydaunorubicin), vincristine sulfate (Oncovin), and prednisone for the treatment of non-Hodgkin lymphoma. Cyclophosphamide, methotrexate, and fluorouracil (5FU), also known as CMF, also are used in combination for treatment of breast cancer. Other chemotherapy regimens used in the clinical setting include the Stanford V protocol, which generally includes a two to three month treatment period with a chemotherapy regimen (doxorubicin, vinblastine, mechlorethamine, vincristine, bleomycin, etoposide, and prednisone) followed by radiation treatment; and the BEACOPP regimen, which includes administering a combination of bleomycin, etoposide, doxorubicin hydrochloride (Adriamycin), cyclophosphamide, vincristine (Oncovin), and procarbazine for treatment of Hodgkin lymphoma. The BEACOPP treatment generally includes four to eight 14- or 21-day cycles with no drugs given on days 15-21. A course of BEACOPP therapy also can be combined with ABVD. An exemplary BEACOPP therapy protocol is set forth in Table 7, below:

### Table 7: BEACOPP Regimen

TABLE 7
BEACOPP regimen

| | | | Dose- | | | |
|---|---|---|---|---|---|---|
| | Base | | Escalated | | | Cycle |
| Drug | BEACOPP | | BEACOPP | | Method | Day |
| (B)leomycin | 10 | mg/m² | 10 | mg/m² | i.v. push | day 8 |
| (E)toposide | 100 | mg/m² | 200 | mg/m² | i.v. | days 1-3 |
| | | | | | infusion | |
| (A)driamycin | 25 | mg/m² | 35 | mg/m² | i.v. push | day 1 |
| (doxorubicin) | | | | | | |
| (C)yclo- | 650 | mg/m² | 1250 | mg/m² | i.v. | day 1 |
| phosphamide | | | | | infusion | |
| (O)ncovin = | 1.4 mg/m² | | 1.4 mg/m² | | i.v. | day 8 |
| Vincristine | (max 2 mg) | | (max 2 mg) | | infusion | |
| (P)rocarbazine | 100 | mg/m² | 100 | mg/m² | orally | days 1-7 |
| (P)rednisone | 40 | mg/m² | 40 | mg/m² | orally | days 1-14 |

Previous combination therapies for treatment of cancer, including those described above, thus exist. These combination therapies, however, lack a rotational component of rotational combinatorial therapies and regimens provided herein. Any of the previously described combinations of cancer therapeutics can be modified in accord with the rotational combinatorial therapy described herein. For example, to prevent adverse side effects associated with administration of multiple medications on day one (i.e., 5 medications on day one of the BEACOPP protocol), the protocols can be modified to administer fewer medications in combination, and rotate the combinations of medications, such as, but not limited to, chemotherapeutics.

b. Combination Therapies in Pain Management

Some individuals with chronic pain undergoing long-term treatment with painkillers, such as opioids, can develop decreased responsiveness to the opioids despite dose titration (Knotkova et al., J. Pain Symptom Manage. 38(3): 426-39 (2009)). Opioid rotation is a strategy implemented to increase efficacy of the combination of opiates; one opiate is initially administered and when efficacy begins to wane, the first opiate is replaced by another to optimize clinical outcomes and lessen side effects. This drug “exchange” substitutes one opioid for another where both medications work through a similar pathway, both targeting the mu receptor; the medications are “exchanged” rather than rotated.

With pain treatment, opioids are substituted when efficiency begins to wane, not at predetermined intervals, and based on a “shared decision-making approach” between the treating physician and the patient (Fine et al., J Pain Symptom Manage (2009) 38(3): 418-425). There is no fixed period at which opioids are exchanged, the exchange is based on clinical efficacy which may or may not correlate with the biological process of receptor downregulation.

In contrast, rotational combinatorial therapies described herein exchanges a group of medications for a second group of medications, where the second group targets different pathways or targets than the first group. With opioid rotation, the rotated opioids are the same, and generally is one drug, not a combination of drugs, exchanged for a second drug. Also, in embodiments herein the rotational therapy described herein includes a regularly defined period for the rotation to occur, including regularly defined intervals, to avoid receptor downregulation and to target different receptors.

c. Combination Therapies in Oral Contraception

Treatment with oral contraceptives employs both combinatorial and pulse-dosed pharmacology treatment strategies; treatment with oral contraceptives is not rotational, the different medications are not rotated. Oral contraceptives come in a variety of formulations; some formulations contain both estrogen analogs and progestin, and some contain only progestin; some pills are monophasic, delivering the same dose of hormones each day, and others are multiphasic, where the doses vary each day. Doses of the component hormones also varies among commercial oral contraceptive products.

The monophasic pills employ a strategy that targets the same, one pathway for birth control each month, with a pulse of hormones, a “pulse-based strategy.”

Multiphasic birth control pills have both and estrogen analogs and progestin components with a fixed increasing dose that is given for set periods of time for a set period (i.e., biphasic dosing includes tablets of one strength for 7 to 10 days, then tablets of a second strength for the next 11 to 14 days; triphasic dosing includes tablets of one strength for 5 to 7 days, then tablets of a second strength for the next 5 to 9 days, and then tablets of a third strength for the next 5 to 10 days). The birth control pills target the same estrogen and progesterone receptors.

Thus, birth control pills can contain a combination of two medications and multiphasic birth control pills can increase in dosage over the month. There is a continuous activation of the same receptors (i.e., one or both of estrogen and progesterone receptors) throughout the month, and activation of these same receptors during the next month. Activation of the same set of receptors differs from the rotational combinational therapy described herein because treatment with birth control pills does not contain a rotational element where different combinations of therapeutics (i.e., medications) are administered and rotated to prevent decreased efficacy due to downregulation of receptor activity; each therapeutic in the rotational combinatorial therapy described herein has a different target or mode of action, unlike birth control pills which target the same receptors each month.

Present combination therapies used for treatment of cancer or for pain management or for contraceptive purposes differ from the rotational combinatorial therapy described herein, and can be modified in accord with the rotational combinatorial regimen described herein. For example, efficacy of chemotherapeutic combinations can be improved by applying the rotational combinatorial regimen described herein; chemotherapeutic combinations can be rotated in accord with a specified schedule to target different receptors or pathways to prevent receptor downregulation or decreased efficacy or increased toxicity, such as adverse side effects, which occurs after prolonged continuous use of the single combination.

d. Combination Therapies to Treat Pathogens

Regular use of antibiotics, such as treatment with a monotherapy or combination therapy, eventually leads to antibiotic resistance in the targeted microorganism (i.e., bacterium). Combination therapies are strategies for treatment of bacterial infections to overcome or prevent drug resistance, broaden the antimicrobial spectrum, improve the efficacy, treat multi-drug resistant bacteria, and lower the dose of the individual drugs to reduce the side effects. Combination therapy with multiple antibiotics have been described (Drusano et al. (2014) PLos ONE 9(7).e101311; U.S. Patent Publication No. 2021/0236589).

US Patent Publication No. 2021/0236589 describes selecting combinations of three antibiotics and rotating among the three different-member combinations to essentially eliminate the possibility of developing resistance.

The combinations of such antibiotics can be administered in a rotational combinatorial regimen to ameliorate side effects from the particular antibiotics and to avoid development of antibiotic resistance.

e. Combination Therapies to Treat Alzheimer's Disease

There is no cure for the progressive neurodegeneration and resulting phenotypes that occur in patients with Alzheimer's disease (AD). A wide spectrum of approaches exist for treatment of AD, with the majority focusing on targeting the Aβ peptide to slow disease progression (Galimberti et al., Ther Adv Neurol Disord 4:203-216 (2011)). Medications and management strategies can improve symptoms, albeit temporarily. Aducanumab (sold under the trade name Aduhelm™) is an intravenous infusion therapy that targets beta-amyloid, and is approved to treat AD. Other medications, such as cholinesterase inhibitors, such as, but not limited to, medications sold under the trademarks Aricept®, Exelon®, and Razadyne®, glutamate regulators, such as, but not limited to the medication sold under the name Namenda®), and combinations of a cholinesterase inhibitor and a glutamate regulator (sold under the trade name Namzaric®). Non-cognitive symptoms, such as behavioral and psychological symptoms, can be treated by orexin receptor agonists (such as the medication sold under the trademark Belsomra®). All of the medications have adverse side effects, including one or more of nausea, vomiting, headache, constipation, confusion and dizziness, impaired alertness and motor coordination, worsening of depression or suicidal thinking, complex sleep behaviors, sleep paralysis, and compromised respiratory function.

A combinatorial pharmacological approach has been described to attenuate the microglial activation and chronic inflammation characteristic of AD (McLarnon (2019) Current Alzheimer Research 16: 1007-1017). The anti-inflammatory combination therapy described by McLarnon does not contemplate rotation of the combinations, and includes administration of a cocktail of compounds to modulate inflammatory pathways activated by microglia in response to the proinflammatory AD brain microenvironment. McLarnon does not consider combination therapy for treatment of aspects of AD pathology that are not related to the chronic inflammation, such as pathology that is related to amyloid deposition or other factors, such as amyloid beta activation or altered cell signaling.

Additional medications for inclusion in the rotational combinatorial therapy for treatment of Alzheimer's are set forth in Table 8, below:

TABLE 8
Exemplary Medications for Alzheimer's Treatment

| Drug | Model | Results | Refence(s) |
|---|---|---|---|
| Minocycline | Aβ-injected rat | decrease of COX-2 | Ryu et al., Glia 48: 85-90 (2004) |
| | Aβ-injected rat | confer neuroprotection | Ryu et al., Glia 48: 85-90 (2004) |
| | Aβ-injected rat | inhibit INOS and N species | Ryu et al., Exp Neurol 198: 552-557 (2006) |
| | Aβ-injected rat | decrease BBB permeability | Ryu et al., Exp Neurol 198: 552-557 (2006) |
| | Aβ-injected rat | confer neuroprotection | Choi et al., Neuropsychopharm 32: 2393-2404 |
| | | | (2007) |
| | transgenic mice | inhibit tau aggregates | Noble et al., FASEB J 23: 739-750 (2009) |
| | in vitro | decrease IL-1β, TNF-α, IL-6 | Seabrook et al., Glia 5: 776-782 (2006) |
| | in vitro | decrease caspase 3 | Noble et al., FASEB J 23: 739-750 (2009) |
| Thalidomide | Aβ-injected rat | decrease BBB permeability | Ryu et al., Neurobiol Dis 29: 254-66 (2008) |
| | Aβ-injected rat | decrease TNF-α, inhibit | Ryu et al., Neurobiol Dis 29: 254-66 (2008) |
| | | angiogenesis | |
| | Aβ-injected rat | confer neuroprotection | Ryu et al., Neurobiol Dis 29: 254-66 (2008) |
| | transgenic mice | decrease TNF-α | Tweedie et al., J Neuroinflamm 9: 106 (2012) |
| | transgenic mice | decrease AB plaques and tau | Tweedie et al., J Neuroinflamm 9: 106 (2012) |
| | | protein | |
| | transgenic mice | enhance synaptic function | Tweedie et al., J Neuroinflamm 9: 106 (2012) |
| | transgenic mice | decrease TNF-α, BACE-1, | He et al., Plos One 8: e55091 (2013) |
| | | plaque formation | |
| | in vitro | decrease TNF-α from Aβ42- | Ryu et al., Neurobiol Dis 29: 254-66 (2008) |
| | | stimulated ug | |
| Ibuprofen | transgenic mice | inhibit COX and IL-1β | Lim et al., J Neurosci 20: 5709-5714 (2000) |
| | transgenic mice | activate PPARY | Lim et al., J Neurosci 20: 5709-5714 (2000) |
| | transgenic mice | inhibit COX2, INOS and BACE-1 | Heneka et al., Brain 128: 1442-53 (2005) |
| | transgenic mice | decrease Aβ42 deposits | Heneka et al., Brain 128: 1442-53 (2005) |
| Dapsone | human | increased IL-8 and receptor | Galimberti et al., Ann Neurol 53: 547-48 |
| | | CXCR2 | (2003); Xia et al., Am J Pathol 150: 1267-74 |
| | | | (1997) |
| | human | increased IL-8 and receptor | Ryu et al., J Cell Mol Med 13: 2911-25 (2009) |
| | | CXCR2 | |
| | Aβ-injected rat | CXCR2 antagonist inhibits | Ryu et al., J Cell Mol Med 13: 2911-25 (2009) |
| | | microgliosis | |
| | Aβ-injected rat | CXCR2 antagonist inhibits | Ryu et al., J Cell Mol Med 13: 2911-25 (2009) |
| | | oxidative stress | |
| | Aβ-injected rat | CXCR2 antagonist is | Ryu et al., J Cell Mol Med 13: 2911-25 (2009) |
| | | neuroprotective | |
| | in vitro | IL-8 most upregulated factor in | Walker et al., Neurobiol Aging 22: 957-66 |
| | | Aβ42-stimulated ug | (2001) |
| | in vitro | IL-8 increases TNF-α, IL-6, IL-1β | Franciosi et al., J Neuroimmunol 159: 66-74 |
| | | in Aβ42-stimulated ug | (2005) |

Additionally, these rotational combinatorial treatments for Alzheimer's disease can include the weight loss regimens or weight loss drugs. For example, glucagon-like peptide 1 (GLP-1) receptor agonists, including those described herein, exhibit neurotrophic and neuroprotective effects in amyloid-β (Aβ) toxicity models of Alzheimer's disease (AD). Hence these drugs can be included in a rotational combinatorial regimen for Alzheimer's disease.

f. Combination Therapies to Treat Hypertension

Combination therapies are not generally used to treat hypertension because of concerns about lowering blood pressure to unsafe levels and increased risk of adverse side effects. Generally, if medications are used in combination to treat hypertension, a first medication is administered and a second can be added to the treatment if the first medication is ineffective and/or there are no significant adverse side effects. There are combination therapies have previously been shown to be superior to monotherapy for treatment of hypertension. A combination of losartan at 50-100 mg with hydrochlorothiazide at 12.5-25 mg lowered systolic blood pressure significantly (p<0.001) more than either drug alone (MacDonald et al., J of the American Heart Association DOI: 10.1161/JAHA.117.006986 (2017)).

Combinations of medications for the treatment of hypertension generally include two antihypertensive agents with different mechanisms of action that demonstrate enhanced efficacy compared to either medication alone. The medications can be used at lower dosages than treatment with individual medications, to reduce the risk or severity of adverse side effects. Combinations of drugs used for treatment of hypertension are set forth in Table 9, below:

TABLE 9
Exemplary Medications for Hypertension Treatment Combination
Drugs for the Treatment of Hypertension

| | |
|---|---|
| Medication | Trade Name |

Diuretic combinations

| | |
|---|---|
| Amiloride and hydrochlorothiazide (5 mg/50 mg) | Moduretic |
| Spironolactone and hydrochlorothiazide (25 mg/50 mg, 50 mg/50 mg) | Aldactazide |
| Triamterene and hydrochlorothiazide (37.5 mg/25 mg, 50 mg/25 mg) | Dyazide |
| Triamterene and hydrochlorothiazide (37.5 mg/25 mg, 75 mg/50 mg) | Maxzide |
| Beta blockers and diuretics | |
| Atenolol and chlorthalidone (50 mg/25 mg, 100 mg/25 mg) | Tenoretic |
| Bisoprolol and hydrochlorothiazide (2.5 mg/6.25 mg, 5 mg/6.25 mg, 10 | Ziac |
| mg/6.5 mg) | |
| Metoprolol and hydrochlorothiazide (50 mg/25 mg, 100 mg/25 mg, 100 | Lopressor HCT |
| mg/50 mg) | |
| Nadolol and bendroflumethazide (40 mg/5 mg, 80 mg/5 mg) | Corzide |
| Propranolol and hydrochlorothiazide (40 mg/25 mg, 80 mg/25 mg) | Inderide |
| Propranolol ER and hydrochlorothiazide (80 mg/50 mg, 120 mg/50 mg, | Inderide LA |
| 160 mg/50 mg) | |
| Timolol and hydrochlorothiazide (10 mg/25 mg) | Timolide |

ACE inhibitors and diuretics

| | |
|---|---|
| Benazepril and hydrochlorothiazide (5 mg/6.25 mg, 10 mg/12.5 mg, 20 | Lotensin HCT |
| mg/12.5 mg, 20 mg/25 mg) | |
| Captopril and hydrochlorothiazide (25 mg/15 mg, 25 mg/25 mg, 50 | Capozide |
| mg/15 mg, 50 mg/25 mg) | |
| Enalapril and hydrochlorothiazide (5 mg/12.5 mg, 10 mg/25 mg) | Vaseretic |
| Lisinopril and hydrochlorothiazide (10 mg/12.5 mg, 20 mg/12.5 mg, 20 | Prinzide |
| mg/25 mg) | |
| Lisinopril and hydrochlorothiazide (10 mg/12.5 mg, 20 mg/12.5 mg, 20 | Zestoretic |
| mg/25 mg) | |
| Moexipril and hydrochlorothiazide (7.5 mg/12.5 mg, 15 mg/25 mg) | Uniretic |
| Angiotensin-II receptor antagonists and diuretics | |
| Losartan and hydrochlorothiazide (50 mg/12.5 mg, 100 mg/25 mg) | Hyzaar |
| Valsartan and hydrochlorothiazide (80 mg/12.5 mg, 160 mg/12.5 mg) | Diovan HCT |

Calcium channel blockers and ACE inhibitors

| | |
|---|---|
| Amlodipine and benazepril (2.5 mg/10 mg, 5 mg/10 mg, 5 mg/20 mg) | Lotrel |
| Diltiazem and enalapril (180 mg/5 mg) | Teczem |
| Felodipine and enalapril (5 mg/5 mg) | Lexxel |
| Verapamil and trandolapril (180 mg/2 mg, 240 mg/1 mg, 240 mg/2 mg, | Tarka |
| 240 mg/4 mg) | |

Miscellaneous combinations

| | |
|---|---|
| Clonidine and chlorthalidone (0.1 mg/15 mg, 0.2 mg/15 mg, 0.3 mg/15 | Combipres |
| mg) | |
| Hydralazine and hydrochlorothiazide (25 mg/25 mg, 50 mg/50 mg, 100 | Apresazide |
| mg/50 mg) | |
| Methyldopa and hydrochlorothiazide (250 mg/15 mg, 250 mg/25 mg, | Aldoril |
| 500 mg/30 mg, 500 mg/50 mg) | |
| Prazosin and polythiazide (1 mg/0.5 mg, 2 mg/0.5 mg, 5 mg/0.5 mg) | Minizide |

Adapted from Skolnik et al., Am Fam Physician (2000) 61(10):3049-3056

There are combination therapies that have been employed for treatment of hypertension, including those described above, but not rotational of combinations. The medications can be administered initially as a combination of two drugs, or therapy can begin with one medication and one or more medications is added to the monotherapy. Any of the previously described combinations of medications for treatment of hypertension or medications known to treat the molecular and cellular pathways associated with hypertension can be modified in accord with the rotational combinatorial therapy and regimens as described herein. For example, to prevent adverse side effects associated with administration of multiple medications at once or to target alternative molecular pathways, the protocols can be modified to administer fewer medications in combination, and rotate the combinations of medications (i.e., ACE inhibitors and/or diuretics).

g. Combination Therapies to Treat Parkinson's Disease

Parkinson's Disease (PD) is a progressive neurodegenerative disorder characterized by altered body movements, including tremor, stiffness, slowed movement (bradykinesia), loss of automatic or autonomic movements, and speech changes. PD is not curable; a number of medical treatments are used in the management or improvement of PD symptoms, including levodopa alone or in combination with carbidopa, dopamine agonists, MAO B inhibitors (i.e., selegiline (sold under the trademark Zelapar®), rasagiline (sold under the trademark Azilect®) and safinamide (sold under the trademark Xadago®), Catechol O-methyltransferase (COMT) inhibitors. Entacapone (sold under the trademark Comtan®) and opicapone (sold under the trademark Ongentys®), Anticholinergics, such as, but not limited to benztropine (sold under the trademark Cogentin®), trihexyphenidyl, and amantadine.

Any of the previously described combinations of medications for treatment of PD can be administered in accord with the rotational combinatorial therapy described herein. In other examples, molecular and cellular pathways associated with PD progression and/or symptoms of PD can be targeted with therapeutics for inclusion in the rotational combinatorial therapy herein. For example, therapeutics that target dopaminergic pathways or neurons or neurotransmitters or receptors can be included in the rotational combination therapy. To prevent adverse side effects associated with administration of multiple therapeutics at once or to target alternative molecular pathways, the protocols can be adjusted to administer fewer medications in combination, and rotate the combinations of therapeutics.

h. Combination Therapies to Treat Chronic Obstructive Pulmonary Disease (COPD)

Chronic obstructive pulmonary disease (COPD) is a type of progressive lung disease characterized by shortness of breath and cough. The two classic COPD phenotypes are emphysema and chronic bronchitis. COPD is not curable; a number of medical treatments are used in the management of stable COPD and exacerbations, including bronchodilators, corticosteroids, inhibitors of the enzyme phosphodiesterase-4, and antibiotics. Bronchodilators for treatment of COPD include short-acting beta₂ agonists [SABAs], long-acting beta₂ agonists [LABAs], short-acting muscarinic antagonists [SAMAs], and long-acting muscarinic antagonists [LAMAs].

Previous studies have shown that long-term treatment with a combination inhaler containing inhaled corticosteroids and long-acting beta₂ antagonist is more effective than either agent alone in improving COPD symptoms and in reducing exacerbation frequency. If a dual therapy is not effective, triple therapy with a combination of ICS, LABA and LAMA is indicated. Different, complementary pharmacological mechanisms of action of the three medications improves clinical benefits. For example, a triple fixed-dose combination of extra fine beclomethasone dipropionate (100 μg/puff), formoterol fumarate (6 μg/puff), and glycopyrronium bromide (12.5 μg/puff) administered via a hydrofluoroalkane pressurized metered dose inhaler is superior to fixed ICS/LABA combined therapy and also superior to the LAMA tiotropium for improving lung function and exacerbation prevention in COPD patients at risk of exacerbation.

Thus, there previously are combination therapies used for treatment of diseases, disorders, and conditions, including those described above. Ultimately, this combination will not avoid the problems associated with desensitization to long-term treatment. These combination therapies can be improved by rotating combinations of the drugs. Any of the previously described combination therapies, where there are different targets for therapeutic intervention or can be modified by rotating combinations of therapeutics or treatments to produce a rotational combinatorial therapy regimen described herein. For example, to prevent receptor downregulation upon continuous administration or adverse side effects associated with administration of multiple medications on one day, the protocols can be modified to administer fewer medications in combination, and rotate the combinations of medications.

i. Combination Therapies to Treat Obesity-Associated Diseases and Conditions

Rotational combinatorial therapy, such as rotational combinatorial pharmacology provided herein can be used to treat diseases, disorders and conditions that are comorbid with overweight and obesity, including, but not limited to, metabolic syndrome, obstructive sleep apnea, non-alcoholic fatty liver disease, diabetes (e.g., diabetes mellitus type 2), cardiovascular disease (e.g., heart attack, stroke), elevated blood pressure, elevated blood cholesterol, and elevated triglyceride levels, and others.

Combinatorial therapy and rotational combinatorial therapy, such as rotational combinatorial pharmacology provided herein can be used to treat cardiovascular disease. Rotational combinatorial therapy can be used in the treatment of cardiovascular diseases including ischemia reperfusion injury resulting from stroke, myocardial infarction, cardiopulmonary bypass, coronary artery bypass graft, angioplasty, or hemodialysis. Rotational combinatorial therapy also can be used in the treatment of the inflammatory response associated with cardiopulmonary bypass that can contribute to tissue injury. For example, a combination of therapeutics can be administered in accord with a regimen described herein prior to, or in order to prevent cardiovascular disease or an adverse cardiac event. In other examples, a combination(s) of therapeutics can be administered in accord with a regimen described herein subsequent to a cardiac event to prevent further injury from the cardiac event or to aid in recovery from the adverse cardiac event (e.g., heart attack or stroke). In one example, a combination(s) of therapeutics can be administered to a subject in accord with a regimen described herein in order to ameliorate the symptoms of cardiovascular disease or the secondary damage or effects resulting from cardiovascular disease. Amelioration of symptoms can be assessed by methods described herein or those known to the skilled physician.

j. Combination Therapies to Treat Overweight and Obesity

There is a lack of therapeutics for obese patients that also focus on polypharmacy for diabetes, metabolic syndrome, and other associated diseases, disorders, and conditions. The approach to weight loss and muscle development described herein is shown to produce results using pharmacological approaches that are at least as effective as bariatric surgery, but without the risks of surgery. This is described in the following sections and detailed in the working examples.

### E. Combinatorial and Rotational Combinatorial Therapy for Weight Loss

As described in the next sections, obesity is an exemplary condition for which combinatorial therapies, and rotational combinatorial therapies can be designed. Fat can be stored subcutaneously or as viscerally subcutaneous and visceral adipose tissue. Subcutaneous fat lies underneath the skin and is visible, and is the fat that changes after body composition improves (i.e., fat decrease) with cardiovascular resistance training. Visceral adipose tissue cannot always be seen directly and distributes around the organs, making it more dangerous to health and is more strongly associated with metabolic syndrome and diabetes compared to subcutaneous fat.

Visceral fat actively contributes to health because it produces cytokines and immunoregulatory hormones. Excess of cytokines and immunoregulatory hormones are seen with obesity, causing inflammation and increased risk of cardiovascular disease, immune-dysregulation and a negative effect on cells sensitivity to insulin further contributing to diabetes.

Obesity can induce a series of chronic metabolic diseases, such as diabetes, dyslipidemia, hypertension and nonalcoholic fatty liver disease.

### 1. Limitations of Existing Treatments for Weight Loss

Given the limitations of lifestyle interventions and bariatric surgery, pharmacotherapeutic approaches for the treatment of obesity are important options. Development of anti-obesity medications has been slow and ineffective. During the past 20 years, several anti-obesity drugs have been discovered, marketed, and subsequently withdrawn from the market; despite showing efficacy during initial stages of treatment, therapeutics for obesity have been accompanied by adverse side-effects following long-term use.

With the exception of the GLP1R agonist, semaglutide, and the combination medication tirzepatide, which activates the GLP-1 and GIP receptors, the average percent body weight reduction for registered drug treatments varies, but they are in the single-digit range (Bray et al., Lancet 387:1947-1956 (2016)). An improved anti-obesity treatment(s) is needed to correct excess weight while reducing risk for cardiovascular-associated adverse effects and psychological adverse effects. Likewise, an improved treatment for overweight or obesity can decrease risk for obesity-related comorbidities including metabolic syndrome, obstructive sleep apnea, non-alcoholic fatty liver disease, diabetes (e.g., diabetes mellitus type 2), cardiovascular disease (e.g., heart attack, stroke), cancer, elevated blood pressure, elevated blood cholesterol, and elevated triglyceride levels, and others.

Significant and harmful side effects are associated with weight loss therapies. Several limitations on surgery exist, including high cost, and the potential for intra-abdominal abscess formation, thrombosis, dehydration, and type 1 diabetic ketoacidosis. As noted above, pharmaceutical weight loss treatments are not as effective as surgical interventions (i.e., generally do not show high percent weight loss) and increase the risk of adverse side effects, including nausea, vomiting, headache, constipation, confusion and dizziness, impaired alertness and motor coordination, worsening of depression or suicidal thinking, complex sleep behaviors, sleep paralysis, and compromised respiratory function.

The robust escalations in obesity and associated health complications constitute major driving forces for the discovery of targets and for the development of safe and effective weight loss therapeutics. The combinatorial and rotational therapy provided herein is different from previous approaches for obesity and other disease processes because they are designed with combinatorial and rotational pharmacology (CRP) in mind.

### 2. Obesity and the Challenges of Treatment

Treatment for obesity can be challenging because of the multitude of causes. A primary treatment for obesity includes dieting and physical exercise. The combination of dieting and exercising, however, rarely produces sustained weight loss, generally resulting from slow weight regain over time.

Altered eating (e.g., dieting) has been recommended as a behavioral change to increase weight loss. CDC recommends a variety of lifestyle interventions for weight loss, including: calorie restriction; time restricted feeding, where meals are consumed within a limited time window (i.e., 6-8 hours) during the day; alternate-day fasting, where food consumption is unrestricted every other day and minimal or no calories are consumed on the other days; “5:2” eating patterns, where meals are unrestricted for 5 days each week, followed by 2 days of restricted calorie intake; and periodic fasting, where calorie intake is restricted for several days in a row (i.e., 5 days) once per month, and food consumption is unrestricted on the other days.

Increased exercise is recommended as a behavioral change to increase and sustain weight loss. The American College of Sports Medicine (ACSM) and CDC Guidelines propose increasing exercise to augment weight loss. For example, the Guidelines indicate that healthy adults aged 18-65 years should participate in moderate intensity aerobic physical activity a minimum of 30 min on five days per week, or vigorous intensity aerobic activity for a minimum of 20 minutes on three days per week.

Behavioral changes targeting lifestyle changes has been studied extensively, with weight loss typically ranging from 3-10% of bodyweight with 12 months of intensive treatment in research settings. Nonetheless, most of that weight lost is regained within 12 months of initiating a program. Lifestyle modifications can be used alone, or as an adjunct to medical or surgical treatments for overweight and obesity, and for long-term treatment and management. (Jin (2018) JAMA 320(11):1210). There are some metabolic and hormonal counterforces that reduce or reverse weight loss; these include an increase in appetite, reduction in energy expenditure, reduced insulin sensitivity that favors growth of adipocyte size and numbers. For example, weight loss is accompanied by persistent endocrine adaptations that cause an increase in appetite and decrease satiety, and there can be a physiological downregulation of pathways associated with weight loss and metabolism, thereby resisting continued weight loss and conspiring against long-term weight maintenance. This can lead to more robust weight regains as the weight lost progresses further from the “weight thermostat” set by years of overeating and all the offsetting mechanisms being activated.

Lifestyle and behavioral modifications are inadequate to provide long lasting weight loss and significant metabolic changes. Pharmacotherapy is necessary in many cases to assist with the metabolic derangements that occur in obese patients but results, as noted below, are very limited. A variety of weight loss treatments have been developed; exemplary therapeutics and treatments are summarized in the following sections.

### 3. Pharmacological Treatments

Obesity is a chronic degenerative disease that can stem from the rewiring of biological mechanisms that hinder weight loss and promote weight gain. Although lifestyle and behavioral interventions provide moderate efficacy, these strategies are limited by complications in adherence. They can be augmented by surgical intervention and/or pharmacological approaches (Müller TD. et al. Nat Rev Drug Discov 21(3):201-223 (2021)). Bariatric surgery generally is considered the most effective approach to weight loss, but it is expensive and limited to single patients, lacking the reach necessary to treat obesity at a global scale. Pharmacological agents can offer such treatment.

The historical development of anti-obesity medications is challenging because of the limited understanding of the molecular mechanisms that control appetite and adverse effects. Historical anti-obesity drug classes include mitochondrial uncouplers, amphetamines, thyroid hormones, drug cocktails, neuromodulators, lipase inhibitors, cannabinoid receptor antagonists, and gastrointestinal-derived peptides chemically optimized for pharmaceutical use (Müller T D. et al. Nat Rev Drug Discov 21(3):201-223 (2021)). Therapeutic approaches regulate the function of pathways related to energy balance and systemic energy sensing. Mechanisms of action can involve limiting fat absorption or food intake, increase satiety, or facilitate energy expenditure.

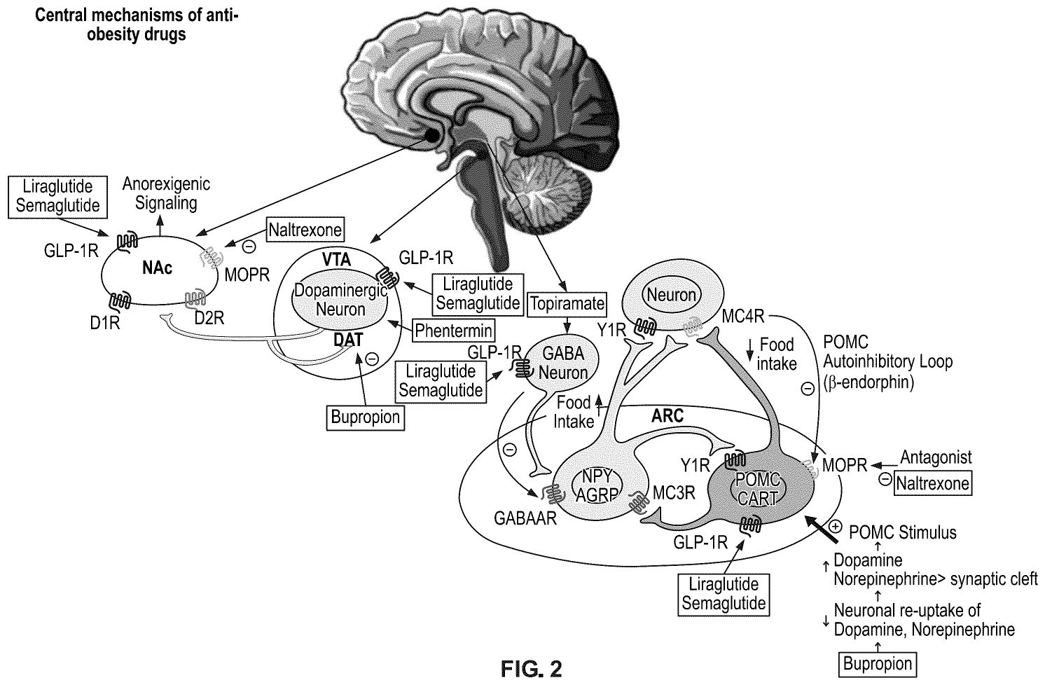

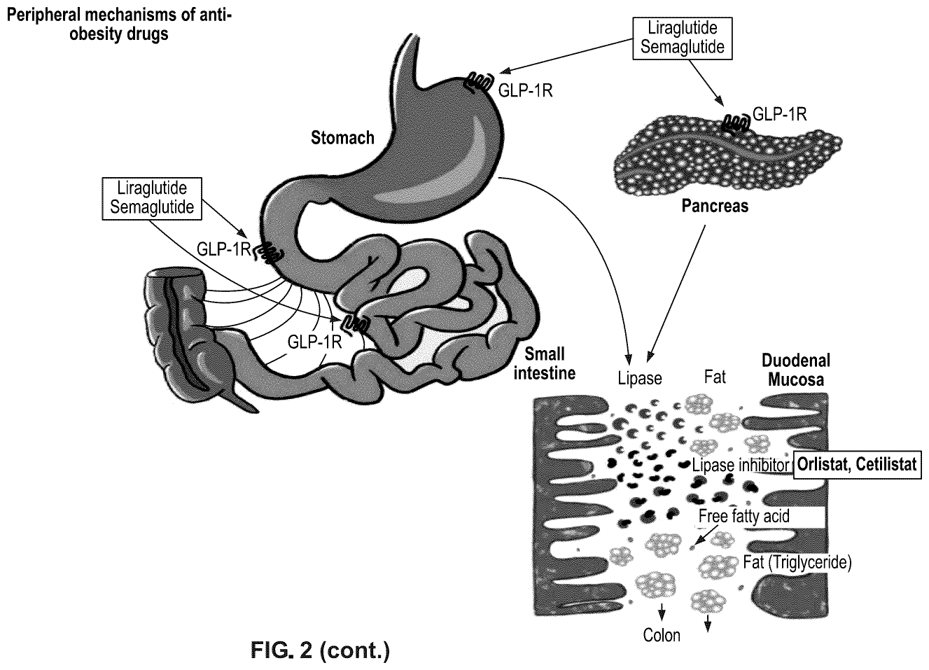

Medical standard of care for weight loss focuses on pharmacological monotherapies and occasionally dual therapies. FDA-approved monotherapy options include phentermine (sold under the trademark Adipex-P®), orlistat (sold under the trademark Xenical®), lorcaserin (sold under the trademark Belviq®), liraglutide (sold under the trademark Saxenda®), phentermine-topiramate (sold under the trademark Qysmia®), naltrexone-bupropion (sold under the trademark Contrave®) and semaglutide (sold under the trademark Wegovy®) medications. These medications, and others, can target components of the central nervous system or peripherally, such as in the gastrointestinal system, including, but not limited to the stomach, small intestine and colon, and pancreas (see e.g., FIG. 2).

As described and exemplified herein, monotherapies have limited efficacy, in part due to the metabolic redundancies and recruitment of alternate and counter-regulatory pathways, and desensitization. Obesity is a disease with multiple etiologies; as described herein can be treated with a multi-targeted approach. As exemplified and described herein, a multi-target approach provides greater benefit than any single medication alone. A multi-targeted approach includes a combination therapy or a rotational combinatorial therapy.

A number of medications have been administered for long-term use for weight loss; these medications, include, but are not limited to, phentermine-topiramate, orlistat, lorcaserin, naltrexone-bupropion, and liraglutide. Treatment with these medications as monotherapies results in weight loss after one year that ranges from 3.0 to 6.7 kg (6.6-14.8 lbs) compared to placebo (Heymsfield et al., (2017) The New England Journal of Medicine 376 (3):254-266). Information on how these drugs affect longer-term complications of obesity, such as cardiovascular disease or death is sparse. obesity drugs do not target weight loss per se, but on appetite suppression and/or are label expansions of other marketed drugs. As a result, treated subjects have significant adverse side effects, and the medications have relatively poor efficacy. For example, semaglutide (sold under the trademark Wegovy®) medication is a therapy designed for treatment of type-2 diabetes. An effect of the drug was weight loss and it has been rebranded for weight loss treatment.

The following are exemplary weight loss drugs that can be combined as described herein for combination therapy and also in a rotational combinatorial therapy regimen for treating overweight, obesity, for effecting weight loss and for treating secondary complications of each of the preceding conditions. The combinatorial therapy can include a combination of therapeutics known to effect weight loss and/or pharmaceuticals previously used as weight loss monotherapies and dual therapies. Medications that previously were used for non-weight loss indications that exhibit weight loss as a secondary effect also can be included in the combination therapies herein. The medications listed herein, such as, for example, below can be included in the combinations herein for treating overweight, obesity and/or for weight loss. Pathways associated with satiation, metabolism, hunger and weight gain or loss can be identified and used to formulate targets and/or therapeutics for the combination therapy, such as therapeutics listed below. Combinatorial regimens employing combinations, such as three or more of the medications below can be included in the combinations for weight loss, and/or treatment of overweight or obesity. These drugs can be part of a rotational combinatorial regimen as well, in which two or more of the drugs are combined and administered for a predetermined time, and then are rotated with another drug or combination of drug for a predetermined time.

a. Amphetamines (e.g., Phentermine-Topiramate)

Amphetamines stimulate norepinephrine release that can result in increased blood pressure, heart rate, and cardiac excitability via binding to vasculature and heart adrenergic receptors. Amphetamines utilized for anti-obesity medication include, but are not limited to Methamphetamine (Desoxyephedrine), Phenmetrazine (sold under the trademark Preludin®), Phendimetrazine, Phentermine, benzphetamine and Diethylpropion. Phentermine (sold under the trademark Adipex-P®) and Diethylpropion (sold under the trademark Tenuate®) were generally designed to retain anorectic activity, but with reduced effects on the cardiovascular and brain reward system (Colman, Ann., Intern. Med. 143, 380-385 (2005)). Several clinical studies report the absence of major adverse effects of phentermine or diethylpropion on blood pressure and heart rate; nonetheless, their use is contraindicated in patients with hypertension or elevated risk for cardiovascular disease (Müller et al., Nat Rev Drug Discov 21: 201-223 (2022)). Patients also quickly develop a tolerance to these medications.

Phentermine is a weight loss medication approved for short term use, such as less than a month (Hendricks et al., Obesity 17:1730-35 (2009)). Patients can rapidly develop a tolerance to the medication.

Amphetamines can be used in combination with other medications for treatment of overweight and obesity. For example, phentermine is used in combination with topiramate (combination sold under the brand name Qsymia®), which can be used for a longer period of time than phentermine alone. Previous studies show that the combination therapy results in an average of 5-10% weight loss, where weight loss corresponds to dosage.

b. Lipase Inhibitors (e.g., Orlistat)

Lipases are a class of digestive enzymes produced in the pancreas, mouth, and stomach to metabolize fat and to facilitate nutrient uptake. Orlistat (sold under the trade name Xenical®) is a lipase inhibitor that reduces the uptake of dietary fat in the gastrointestinal tract. The resulting fat malabsorption after treatment with lipases facilitates a negative energy state leading to a placebo-normalized weight loss in the range of 2.5%. Orlistat has shown beneficial effects on blood glucose, non-alcoholic fatty liver disease, and blood pressure (Müller et al. (2018) Pharmacol. Rev. 70:712-746; Khera et al. (2018) Gastroenterology 154:1309-1319.e7). The most common adverse events after orlistat treatment are of a gastrointestinal or digestive nature.

c. Serotonergic Agonists—Neuromodulators (e.g., Lorcaserin)

Serotonergic agonists act as anti-obesity medication by suppressing appetite through the activation of serotonin receptors. Clinical serotonergic agonists include Fenfluramine, Dexfenfluramine, Sibutramine, Lorcaserin (sold under the trademark Belviq®), and Tesofensine. Cardiovascular safety concerns accompany Fenfluramine, Dexfenfluramine, and Sibutramine (Müller et al. (2018) Pharmacol. Rev. 70:712-746). The FDA requested withdrawal of Lorcaserin due to clinical trials showing an increased occurrence of cancer (Müller et al., Nat Rev Drug Discov 21: 201-223 (2022)). Tesofensine, an inhibitor of norepinephrine, serotonin, and dopamine reuptake that was initially advanced for treatment of Alzheimer disease. A phase II study, it was reported to dose-dependently decrease body weight by 4.4-10.4% (Wharton et al. (2015) J. Curr. Cardiol. Rep. 17:35; Astrup A. et al. (2008) Lancet 372:1906-1913).

d. Bupropion/Naltrexone (Such as the Product Sold Under the Trademark Contrave®)

Naltrexone, an opioid antagonist, blocks the inhibitory effects of opioid receptors activated by β-endorphin released in the hypothalamus, which stimulates feeding. Naltrexone reduces food intake in combination with bupropion, reuptake inhibitor of dopamine and norepinephrine. Naltrexone does not cause weight loss in monotherapy; patients treated with Naltrexone 32 mg plus Bupropion showed a body weight reduction of 6.1% after 56 weeks of treatment (Greenway et al. Lancet 376, 595-605 (2010)). No increased adverse cardiovascular events were noted in an analysis of a cardiovascular outcome trial after bupropion/naltrexone treatment (Nissen et al., JAMA 315:990-1004 (2016)). Another study showed that addition of a modified diet and increased exercise to the bupropion/naltrexone combination lost an average of 9.3% of their baseline body weight.

e. Glucagon-Like Peptide-1 Receptor (GLP1R) Agonists

Glucagon-like peptide-1 (GLP1; SEQ ID NO: 1) acts at the pancreas to enhance the expression and secretion of insulin and to inhibit the release or glucagon. GLP1 and glucose-dependent insulinotropic polypeptide (GIP; SEQ ID NO:10) are primary incretin hormones secreted from the intestine on ingestion of glucose or nutrients to stimulate insulin secretion from pancreatic R cells. Although the specific mechanism of action is multifactorial, with gut, brain, and systemic improvements in insulin sensitivity, GLPR1 antagonism can lead to decreased body weight via the inhibition of food intake (Müller et al. (2018) Pharmacol. Rev. 70:712-746).

Chronic treatment with GLP-1 receptor agonists causes weight loss in diabetic humans. Liraglutide (sold under the trademark Saxenda®), is a once-daily injectable GLPR1 agonist, and was the first GLP1R agonist approved for treatment of obesity. Liraglutide is short-acting, so it is administered daily. After one year of treatment, there was a reported mean decrease of 8% body weight in subjects treated with liraglutide compared to 2.6% in subjects treated with vehicle controls (Pi-Sunyer et al. (2015) N. Engl. J. Med. 373:11-22). In 2020, treatment with 3 mg liraglutide was approved for weight management in adolescents with obesity.

GLP-1 increases insulin metabolism and plays a role in appetite and digestion. It is among the incretins, which are hormones released by the small intestine into the bloodstream following a meal to help lower blood sugar by triggering insulin and blocking other sugar sources. Medications that are GLP-1 agonists are referred to as incretin mimetics since they “mimic” the incretin hormone effects.

Semaglutide, which is similar to and acts like a GLP-1 hormone, is used as an anti-diabetic medication. Semaglutide also slows down the rate at which food leaves the stomach (called gastric emptying). These actions cause a feeling of fullness, lowering appetite and resulting in weight loss. Semaglutide (sold under the brand name Wegovy®) is an injectable GLPR1 agonist FDA-approved in 2021 for chronic weight management in adults with obesity (URL: fda.gov/news-events/press-announcements/fda-approves-new-drug-treatment-chronic-weight-management-first-2014). Wegovy® weight loss medication is approved for treatment of adults with a body mass index (BMI) greater than or equal to 30 mg/kg² alone or 27 mg/kg² with at least one weight-related comorbidity (e.g., high blood pressure, high cholesterol). Semaglutide administered at a dose of 2.4 mg lowers mean body weight to ˜12-15% after 68 weeks of treatment (relative to ˜2.4% in placebo controls) with once-weekly treatment (Wilding et al., N. Engl. J. Med. 384(11): 989-1002 (2021)). A longer mechanism of action compared to other weight loss medications allows semaglutide to be administered weekly; semaglutide is the first once-weekly GLP-1 agonist approved for weight loss. Daily dosing of 2.4 mg achieved >15% weight loss in half of the study participants, whereas one third experienced more than a 20% reduction (O'Neil et al., Lancet 392:637-649 (2018)). Semaglutide is well-tolerated although the adverse effects (nausea, diarrhea, vomiting and constipation) typified by GLP1-related remain. The high level of side effects can limit a patient's ability to maintain or reach a target level of the drug. In such cases, a provider can extend the period to reach the target dose, or may have to discontinue treatment if the patient is unable to tolerate the target dose, due to the adverse side effects.

Several peptide and small-molecule GLP1R agonists are in clinical development, including formulations designed for oral administration (Müller et al., Nat Rev Drug Discov 21: 201-223 (2022)). GLP1R agonists in clinical development include Efpeglenatide (s a long-acting GLP-1RA that is a single amino acid-modified exendin conjugated to a fragment crystallizable region of human immunoglobulin 4 via a 3.4-kDa mini-polyethylene glycol linker), semaglutide sold under the trademark Rybelsus®, Danuglipron (formerly PF-06882961), GLPR-NPA, and lotiglipron (formerly PF-07081532). Several GLP-1 receptor agonists approved to improve glycemic control and lower blood sugar in adults with type 2 diabetes exhibit a secondary effect of increased weight loss. For example, type 2 diabetes patients taking ≥1500 mg/day metformin in combination with the GLP-1 receptor agonist sold under the tradename dulaglutide, sold under the tradename Trulicity®, at 1.5 mg, 3.0 mg, or 4.5 mg once weekly lost an average of 6.6, 8.4 and 1.1 pounds, respectively, after 36 weeks on the medication.

f. Mitochondrial Uncouplers

An alternative approach to decreasing food intake and/or absorption is to increase the metabolic efficiency by which food is converted into useful energy. Highly thermogenic tissues such as brown adipose tissue show high expression of Uncoupling Protein 1 (UCP1, SEQ ID NO:41) that uncouples oxygen consumption from ATP synthesis. Pharmacological upregulation of UCP1 activity can be induced by catecholamines or small molecule uncouplers. Small molecule uncouplers for anti-obesity application include 2,4-dinitrophenol (DNP) and BAM15 ((2-fluorophenyl){6-[(2-fluorophenyl)amino](1,2,5-oxadiazolo [3,4e]pyrazin-5-yl)}amine). DNP is the first small molecule uncoupler tested clinically, but has limited utility owing to toxicity. Controlled-release oral formulation of DNP are under investigation to achieve an enhanced therapeutic index (Müller et al., Nat Rev Drug Discov 21: 201-223 (2022)). BAM15 is an orally administered therapeutic that reverses diet-induced obesity and insulin resistance in mice (Alexopoulos et al., Nat. Commun. 11(1): 2397 (2020)).

g. Thyroid Hormones

Thyroid hormone can decrease body weight and body fat by stimulating energy expenditure (Müller et al., Pharmacol. Rev. 70:712-746 (2018)). Thyroid hormone also can improve hepatic lipid metabolism and decrease low-density lipoprotein (LDL) cholesterol via enhanced reverse cholesterol transport and clearance of LDL via the liver (Baxter et al., Nat Rev Drug Discov 8:308-320 (2009)). The biologically active form of thyroid hormone is tri-iodothyronine (T3), which promotes its pharmacology through two specific nuclear thyroid receptor (TR) isoforms, TRα and TRβ. Administration of T3 increases metabolic rate in a variety of species, including mice, rats, and humans (Müller et al., Pharmacol. Rev. 70, 712-746 (2018)). The molecular mechanism underlying T3 modulation of metabolic rate includes uncoupling of oxidative phosphorylation from mitochondrial ATP synthesis in skeletal muscle and other peripheral tissues, regulation of lipogenesis, activation of Na+/K+ ATPase, enhanced mitochondrial biogenesis, and stimulation of futile cycling (Müller et al., Pharmacol. Rev. 70:712-746 (2018)). Excess thyroid hormone also leads to muscle and bone catabolism, as well as several cardiovascular adverse effects including cardiac arrhythmia, tachycardia, and heart failure, severely limiting its use as a weight loss therapeutic.

h. Drug Cocktails

Metabolic redundancies and recruitment of alternate and counter-regulatory pathways can limit the efficacy of monotherapies. Clarkotabs, developed in 1941, were among the first commercially distributed combination diet pills; the combination sought to harness the anorectic effect of amphetamines with the thermogenic effect of thyroid hormone, with Aloin and Atropine sulfate to counteract potential adverse cardiovascular effects (Müller et al., Pharmacol. Rev. 70:712-746 (2018)). Later combinations included cocktails of weight-reducing substances, including d-amphetamine or related analogs (e.g., Diethylpropion, Fenfluramine, Sibutramin, or Fenproporex), thyroid hormones, diuretics, laxatives, Chlorthalidon, Ephedrine, and/or Phenolphthalein. Substances such as Digitalis, Belladonna, benzodiazepines, barbiturates, corticosteroids, cardiac glycosides, beta-blocker, and potassium were common additives used to counteract or mask adverse cardiovascular effects of the drug cocktail (Cohen PA. et al., Am J Public Health 102:1676-1686 (2012)).

The combination of the amphetamine phentermine and topiramate was approved by the FDA in 2012. Topiramate is a sulfamate-substituted monosaccharide derived from D-fructose, commonly used to treat epilepsy and migraine. Although the mechanism of how the combination improves systemic metabolism is unknown, placebo-normalized weight loss ranges from 5.9%-9.6% (Müller et al., Pharmacol. Rev. 70:712-746 (2018)).

The weight loss combination of the medications naltrexone and bupropion combines two medications approved for indications other than weight loss. Bupropion, a dopamine-norepinephrine reuptake inhibitor is approved to treat depression and aid in smoking cessation, and naltrexone, an opioid receptor antagonist is approved to treat alcohol and opioid dependence. The naltrexone-bupropion combination is marketed under the trade name Contrave® and is FDA approved for chronic weight management.

i. Cannabinoid Receptor Antagonists

Rimonabant, an endocannabinoid 1 receptor (CB1) antagonist, acts by modulating neurons in both homeostatic and hedonic feeding circuits, leading to placebo-subtracted weight loss of ˜2.6-6.3 kg (Müller et al., Pharmacol. Rev. 70: 712-746 (2018); Pi-Sunyer et al. JAMA 295:761-775 (2006)). Rimonabant was discontinued in 2009 due to serious adverse psychiatric effects (Sam et al., J Obes 2011:432607 (2011)).

j. GIPR Agonists and GIPR/GLP1R Combination Agonists

Glucose-dependent insulinotropic polypeptide (GIP; SEQ ID NO:10) is a hormone involved in blood sugar control. GIP receptor (GIPR) agonists have been shown to decrease food intake, increase energy expenditure, decrease body weight and to improve glucose handling in preclinical studies (Mroz et al., Mol. Metab. 20:51-62 (2019); Zhang et al., Cell Metab. 33:833-844.e5 (2021)). GIP is known to be upregulated following Rou-en-Y gastric bypass.

Body weight loss associated with GLP-1 agonist treatment is enhanced when GLP-1 and GIP are co-administered (Matthias Tschöp oral presentation at ADA (American Diabetes Association), 2011; Tschöp et al., Diabetes 66:1766-1769 (2017)). GIPR/GLP1R dual agonists and co-administration of GLP-1R agonists and GIPR agonists have demonstrated metabolic benefits and reduced body weight in mice when compared to GLP1R agonists (Coskun et al., Mol. Metab. 18: 3-14 (2018); Finan et al., Sci. Transl Med. 5, 209ra151 (2013)). Thus, in addition to improving blood glucose control, GIP can also enhance GLP-1-mediated body weight loss. GIP/GLP1 dual agonists include Tirzepatide, GIP/GLP peptide I, GIP/GLP peptide II, and NN9709.

Tirzepatide is a combination medication that activates the GLP-1 and GIP receptors; tirzepatide contains GLP-R and GIPR agonists). Tirzepatide (available under the trademark Mounjaro™) is a dual glucose-dependent insulinotropic polypeptide (GIP) and glucagon-like peptide-1 (GLP-1) receptor agonist. The drug is manufactured by Eli Lilly & Co. and was approved in May 2022. Tirzepatide works similarly to the other drugs in the GLP-1 receptor agonist family, but it has additional effects that appears to give it a slight edge. It has a dual-action design, mimicking the action of two incretin hormones involved in blood glucose control: Glucagon-like peptide-1 (GLP-1) and glucose-dependent insulinotropic polypeptide (GIP). This is the first-in-class medicine to act on both of these receptors.

Tirzepatide is a 39 amino acid peptide GLP-1 receptor agonist based on the GIP sequence. It contains 2 noncoded amino acids in positions 2 and 13, a C-terminal amide, and a lysine residue at position 20 that is attached to a 1, 20 eicosanoic acid via a linker. Studies of tirzepatide were conducted through two placebo-controlled trials (SURPASS-1 and -5), three trials in combination with metformin, sulfonylureas and/or SGLT2 inhibitors (SURPASS-2, -3, -4), and two additional trials conducted in Japan. A phase III trial of tirzepatide in patients with type 2 diabetes of excess weight revealed a body weight reduction of >15% relative to 9% in patients treated with 1 mg semaglutide (Frias et al., N. Engl. J. Med. 385, 503-515 (2021); URL: fda.gov/news-events/press-announcements/fda-approves-novel-dual-targeted-treatment-type-2-diabetes; published May 13, 2022).

A separate study looking at efficacy of tirzepatide (5 mg, 10 mg, 15 mg) was conducted via the SURMONT-1 clinical trial, where participants received once a week GLP-1 receptor agonist. Patients receiving the GLP-1 receptor agonist lost up to 22.5% (52 pounds or 24 kg) of their body weight. The study enrolled 2,539 participants across the US, Argentina, Brazil, China, India, Japan, Mexico, Russia, and Taiwan and was the first phase 3 global registration trial evaluating the efficacy and safety of tirzepatide in adults with obesity, or overweight with at least one comorbidity, who do not have diabetes. Study participants were assessed for mean percent change in body weight at week 72 and the percentage of patients achieving ≥5% weight loss from baseline to week 72 as primary end points. Secondary end points included the percentage of patients achieving greater than or at 10 and 20% weight loss from baseline to week 72. The study results are set forth in the table below:

| | Estimated | Patients with | Body weight |
|---|---|---|---|
| Doses and | mean weight | weight loss | change from |
| comparators | change | of >5%, >20% | baseline (kg) |
| SC tirzepatide 5 mg | −15% | 85%, 30% | −16 |
| SC tirzepatide 10 mg | −19.5% | 89%, 50% | −22 |
| SC tirzepatide 15 mg | −20.9% | 91%, 57% | −24 |
| Placebo | −3.1% | 31%, 3.1% | −2 |

The study results show that participants taking tirzepatide achieved average weight reductions of 16.0% (35 lb. or 16 kg on 5 mg), 21.4% (49 lb. or 22 kg on 10 mg) and 22.5% (52 lb. or 24 kg on 15 mg), compared to placebo (2.4%, 5 lb. or 2 kg). Additionally, 89% (5 mg) and 96% (10 mg and 15 mg) of people taking tirzepatide achieved at least 5% body weight reductions compared to 28% of those taking placebo. Tirzepatide met both co-primary endpoints of superior mean percent change in body weight from baseline and greater percentage of participants achieving body weight reductions of at least 5% compared to placebo. The study also achieved all key secondary endpoints at 72 weeks. There was no data on actual fat loss or muscle mass loss or retention. Safety and adverse reactions also were assessed; the investigators found that 18%, 17%, 9%, 7% and 5% of trial participants experienced nausea, diarrhea, vomiting, constipation, and abdominal pain, respectively.

Tirzepatide (sold under the trademark Mounjaro™) was approved by the US Food and Drug Administration as a once-weekly subcutaneous injection to treat adults with type 2 diabetes, to improve blood sugar levels. As detailed above, and herein, Tirzepatide showed significantly better long-term blood sugar control (i.e., A1C) and weight loss compared to semaglutide.

Glucagon and glucagon agonists have been shown to promote satiety and to increase energy expenditure when used in combination in both rodents and human (Kleinert et al., Int J Mol Sci. 20(21): 5407 (2019)). A triagonist peptide that act as an agonist of the glucagon-like peptide-1 (GLP-1), glucose-dependent insulinotropic polypeptide (GIP) and glucagon receptors was previously described to effect weight loss in animal models (Finan et al., Nat Med 21(1):27-36 (2015)).

k. GLP1R/Glucagon Dual Agonists

GLP-1 receptor (GLP1R) agonists in combination with glucagon (GcgR) agonists participate in several mechanisms of body weight reduction, including appetite suppression, thermogenesis and lipolysis, while minimizing the risk of hyperglycemia (Müller et al., Nat Rev Drug Discov 21(3):201-223 (2021)). Combination strategies of these agonists have included Cotadutide (MEDI0382), BI 456906, Efinopegdutide (^{LAPS}GLP/GCG), and oxyntomodulin (OXM; SEQ ID NO:4). Several GIP/GLP1/glucagon tri-agonists also have been developed for weight loss, including HM15211 (^{LAPS}Triple Agonist), GGG tri-agonist, LY3298176 and NN9423.

l. Summary

With few exceptions, the field of pharmaceutically assisted weight loss has focused on monotherapy and the most effective pharmaceuticals include versions of a GLP-1 agonist. Although approved for use in human, several therapeutics that show efficacy are not widely used because of adverse side effects or toxicity associated with administration of the medications at the therapeutic or FDA approved dosages. For example, patients taking Rimonabant experience adverse psychiatric effects, patients taking thyroid hormones and amphetamines experience adverse cardiac effects, patients taking lorcaserin show increased occurrence of cancer, and patients taking orlistat experience gastrointestinal issues.

A combination of therapeutics administered in accord with the rotational protocol/regimen described herein incorporates pharmacological principles known to occur after a gastric bypass, as well as overcoming tolerance and/or desensitization to medications due to receptor downregulation/desensitization to medications. The combination therapy can be administered at a lower dosage than the monotherapeutic dosage to decrease side effects associated with administration of the monotherapy. The combination therapy is rotated at intervals (i.e., monthly, bi-monthly, or tri-monthly) wherein the patient is not administered an individual therapeutic for an extended period; the finite time period for administration of the medication decreases the time for developing adverse side effects (i.e., severe adverse effects) or downregulation of the pathways activated by the medications.

The rotational combinatorial therapy described herein can include a combination of a plurality of different known therapeutics and/or treatments. For example, the combination for treatment of obesity or for weight loss can include more than one previously characterized weight loss treatment. In some examples, the rotational combinatorial therapy can include one or more FDA-approved monotherapy options, such as, for example, phentermine (sold under the trademark Adipex-P®), orlistat (sold under the trademark Xenical®), lorcaserin (sold under the trademark Belviq®), liraglutide (sold under the trademark Saxenda®), phentermine-topiramate (sold under the trademark Qysmia®), naltrexone-bupropion (sold under the trademark Contrave®), tirzepatide (sold under the trademark Mounjaro™), and semaglutide (sold under the trademark Wegovy®) medications. The rotational combinatorial therapy provided herein also can be used for treatment of any condition with a previously characterized mechanism of action or pathway, where a combination of therapeutics can be developed for treating the disease or disorder or condition. For example, a treatment of obesity or overweight, for which a plurality of different known molecular targets for treatment or multiple known molecular or cellular pathways are known to be involved in disease progression; a combinatorial therapy including one or more of amphetamines, lipase inhibitors, serotonergic agonists, opioid agonists, Glucagon-like peptide-1 receptor (GLP1R) agonists, mitochondrial uncouplers, thyroid hormones, diuretics, dopamine-norepinephrine reuptake inhibitors, cannabinoid receptor antagonists, GIP agonists, GIPR agonists, and GLP1R/glucagon dual agonists can be administered in a rotational combinatorial therapy to target multiple pathways that are associated with a disease or disorder, but where a pharmacotherapy has not yet been developed.

### 4. Surgical Treatments

The most effective treatment for obesity has been bariatric surgery (Kissler et al., (2013) Semin Nephrol. 33(1):75-89). Bariatric procedures include, for example, laparoscopic adjustable gastric banding, Roux-en-Y gastric bypass, vertical-sleeve gastrectomy, and biliopancreatic diversion. Surgery for severe obesity is associated with long-term weight loss, improvement in obesity-related conditions, and decreased overall mortality. A resolution of co-morbidities, such as diabetes, hypertension, fatty liver, urinary distress, and hyperthyroidism, also have been shown to occur following bariatric surgery (Susmallian et al., Medicine 98(3): e13824 (2019)).

Weight loss of between 12% and 35% (depending on the type of procedure performed) is expected at 1-2 years after surgery. The average weight loss in clinical practice after 2 years for adjustable gastric banding is about 20%, weight loss after Roux-en-Y gastric bypass is about 30%, and weight loss after biliopancreatic diversion or duodenal switch is about 35% (Cummings et al., (2004) J Clin Endocrinol Metab 89:2608-2615). Complications occur in about 17% of cases and reoperation is needed in 7% of cases (Chang et al., JAMA Surgery (Meta-analysis, Review) 149 (3): 275-87 (2014); doi:10.1001/jamasurg.2013.3654).

Weight loss after bariatric surgery results, not only from the bypass and other physical changes, but also from factors other than the physical changes to the gastrointestinal tract and calorie reduction following surgery (Cummings et al., J Clin Endocrinol Metab 89:2608-2615 (2004)). Post-surgical hormonal alterations occur that can change activation of brain pathways that regulate appetite cues (Batterham et al., Nature 450:106-109 (2007)).

Vagal nerve stimulation (VNS) delivers short bursts of energy into the vagus nerve in the brain, is used for treatment of drug-resistant epilepsy, and has been associated with weight loss. VNS has been associated with weight loss (>5% of body weight) within 6-12 months of stimulation. The weight loss is due to the action of afferent fibers of the nerve which participate in the brain-gut axis, which take part in a feedback loop induced by the presence of food in the gastrointestinal tract inducing hypometabolism of the hypothalamus and subsequent involvement of the satiety centers, thereby controlling food intake.

Because of the high monetary costs of surgery, the extensive and taxing lifestyle modifications, and substantial risks, effective and less invasive treatments are needed. As described herein, the benefits of surgical intervention, can be achieved by applying the combinatorial, and rotational combinatorial methods provided and described herein for weight loss by selecting and administering combinations of drugs/treatments to mimic the hormonal and physiological effects of surgery. The combinations of therapeutics also can be rotated.

### F. MODIFICATIONS AND ENHANCEMENTS OF PHARMACOLOGICAL WEIGHT LOSS TREATMENTS TO IMPROVE CLINICAL OUTCOMES

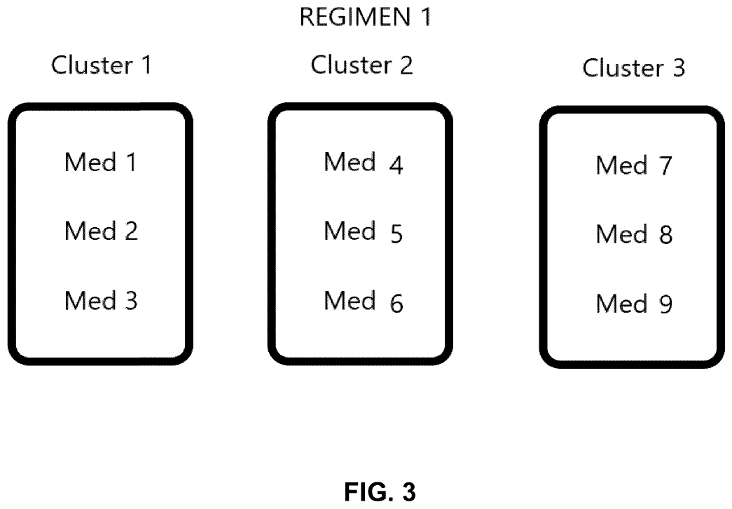

Complex and redundant systems control food intake and energy expenditure (see Lenard et al., Obesity (Silver Spring) 16 (Suppl 3): S11-22 (2008). Pharmaco-therapy treatments and candidates under clinical development for treatment of overweight and obesity can be characterized, for example, as follows: 1) medications that act peripherally to impaired dietary fat absorption; 2) medications that act centrally to decrease food intake; and 3) medications to facilitate energy expenditure (see e.g., FIG. 3). Medications that increase muscle development also are used. Despite compliance with weight loss guidelines, many drugs launched for the treatment of obesity over the last two decades have been withdrawn due to safety issues associated with increased risk of cardiovascular or psychiatric complications. The failure of these medications can be attributed to the multifactorial pathogenesis and the complex neurohormonal regulation of energy balance. Although monotherapy provides some efficacy for treatment of a disease, disorder, or condition where the target is a single protein in a pathway involved in obesity, physiological counter-regulatory mechanisms involving alternate pathways pose imitations. Consequently, as described herein a disease with multiple targets and/or that involves multiple pathways and/or etiologies requires a multi-targeted approach. Obesity is exemplary of such diseases, disorders, and conditions in which multiple pathways are involved. This approach can be applied to other chronic diseases, disorders, and conditions that involve a plurality of targets/pathways/etiologies. These methods also are for treating diseases, disorders, and conditions for which the subject becomes desensitized to treatment, requiring higher doses and consequent increased risk of adverse effects.

As exemplified by treatment of obesity, the combinatorial therapy and rotational combinatorial therapies provided herein correct excess weight while reducing risk for adverse effects (i.e., cardiovascular and psychological adverse effects) and without increasing muscle loss by taking into account the multifactorial processes associated with metabolism and weight gain. Combinations for use in the combinatorial and rotational combinatorial therapies provided herein include a plurality of medications and/or treatments. The combinations can be rotated to overcome downregulation that occurs after prolonged monotherapy. The combinations target the multifactorial processes involved in weight gain or failure to lose weight. The rotation prevents or mitigates the desensitization/downregulation that occur upon long-term exposure to some of the weight loss therapeutics. The rotational combinatorial therapy herein includes combinations of medications that: act peripherally to impaired dietary fat absorption; act centrally to decrease food intake; facilitate energy expenditure; and increase muscle development.

The combinatorial, and the rotational combinatorial therapies described herein for treating overweight or obesity also can decrease risk for obesity-related comorbidities including metabolic syndrome, obstructive sleep apnea, non-alcoholic fatty liver disease, diabetes (e.g., diabetes mellitus type 2), cardiovascular disease (e.g., heart attack, stroke), cancer, elevated blood pressure, elevated blood cholesterol, and elevated triglyceride levels, and others. As a non-surgical option for treating obesity and overweight, the combination and rotational combinatorial therapy described herein is not associated with the harmful side effects associated with surgery, such as high cost, and the risk of developing intra-abdominal abscess, thrombosis, dehydration, and type 1 diabetic ketoacidosis.

### 1. Developing Combination and Rotational Combinatorial Therapies for Weight Loss

Developing a combinatorial therapy and rotational combinatorial therapy for weight loss, such as for use for treating overweight or obesity, includes assessing a variety of factors, selecting patients for treatment, selecting molecular and/or cellular pathways for treatment, selecting therapeutics for treatment, and developing the rotational regimen, including timing for administration and timing for rotating the combinations, and dosages. For example, molecular and/or cellular pathways known to contribute to weight loss or appetite regulation or metabolism, or molecular and/or cellular pathways that are associated with weight loss or appetite regulation or metabolism can be targeted for therapy. For example, peptides or proteins that are associated with molecular and/or cellular pathways that contribute to or impact weight loss, appetite regulation, and/or metabolism can be selected for use for treatment of overweight or obesity, or to effect weight loss. The combinations of treatments are identified based on these criteria and those discussed below to combine treatments that, for example, mimic the effects of bariatric surgery. The combinations (or clusters) can be rotated to produce rotational combinatorial protocols to avoid desensitization and/or receptor downregulation.

Several factors can be assessed for developing combinatorial and rotational combinatorial therapies for weight loss. For example, peptides known to contribute to weight loss and/or appetite regulation and/or metabolism can be assessed and included in the combinations. In determining pathways to target for weight loss and peptides to include in the combinations, the following can be assessed: 1) the pharmacodynamic and pharmacokinetic properties of each peptide individually and in combinations with other peptides and/or other therapeutics; 2) stability in serum, to ensure adequate stability for the intended use; 4) solubility of the peptides and/or other therapeutics; the delivery profile to ensure activity, stability, solubility and other characteristics to ensure therapeutic activity and efficacy.

### 2. Pathways to Target for Weight Loss

Research on new signaling molecules has substantially increased the knowledge of central and peripheral mechanisms underlying homeostatic energy balance. Homeostatic mechanisms involve multiple components including neuronal circuits, some originating in hypothalamus and brain stem, as well as peripherally derived satiety, hunger and adiposity signals that modulate neural activity and regulate eating behavior. Dysregulation of one or more of these homeostatic components can result in or contribute to obesity.

Studies of gastric bypass patients show that the surgical procedure, where the stomach is reduced in size and consequent caloric restriction and malabsorption, is not solely responsible for the subsequent weight loss; (see, e.g., Vincent and Roux (2008) Clinical Endocrinology 69(2): 173-179; Korner et al., Obesity 14:1553-1561 (2006)).

For example, after a form of gastric bypass surgery, Roux-en-Y gastric bypass (RYGB), 84% of patients who had type-2 diabetes showed complete remission of this disease, and almost all had improved glycemic control. Type 2 diabetes symptoms resolve quickly after surgery and before weight loss, and patients exhibit improved glucose homeostasis after RYGB compared to similar weight loss achieved via other means (i.e., diet and/or exercise). These rapid changes indicate that the resultant weight loss and/or reduced caloric intake, does not explain these weight-independent antidiabetic actions of RYGB.

Post-surgery changes in several of these pathways and others, including gastro-intestinal hormones, including increases in GLP-1, PYY, and oxyntomodulin, decreases in GIP and ghrelin, and a combination of these changes modulate initial and long-term maintenance of weight loss (see e.g., Ochner et al., Int J Obes 35(2):153-166 (2011)). Hormone profiling of subjects after bariatric surgery provides the following insights: 1) weight loss is an integrated hormonal process, 2) multiple peptides are involved (Combination) and 3) peptides have fluctuations and in timing and release (Rotation).

Peptide levels that change after gastric bypass contribute to weight loss following the surgery. In accord with methods herein, peptides and/or other therapeutics, including peptide/receptor agonist or antagonists, depending on the target, in combinations that mimic the fluctuations of peptides whose expression or activity is altered after gastric bypass or other weight loss surgery. The combination therapy herein takes into account the integrated processes that occur during weight loss, and seek to mimic the multiple pathways using combinations of therapeutics administered to mimic the fluctuations in timing and release that occur following gastric bypass. A combinatorial therapeutic regimen is described because multiple pathways are involved and because monotherapies are generally ineffective or less effective than gastric bypass. The combination therapies described herein administered with a rotational approach overcome desensitization to the peptides and/or therapeutics that can occur due to receptor downregulation, which occurs over time after monotherapy or long-term combination therapy.

Weight loss surgery patients also experience an increase in muscle mass following surgery; therapeutic agents that alter pathways that affect (i.e., increase) muscle generation to increase muscle mass during the weight (fat) loss protocol. Weight loss surgery patients also experience a decrease in muscle mass following surgery, and it is provided herein to provide such patients with agents that alter pathways that affect (i.e., increase) muscle generation to increase muscle mass during the weight (fat) loss protocol. Obesity is associated with an increased risk of developing insulin resistance and type 2 diabetes. The therapy described herein can include peptides to decrease insulin resistance that can be a comorbidity with overweight or obesity.

For example, in obesity, more than over 20 peptides and hormones are known to orchestrate the cellular milieu. In any of the examples of the combination and rotational combinatorial therapies provided herein the combinations can include peptides that are associated with metabolism, satiety, physiological response to hunger or food ingestion, and/or digestion. These combinations include peptides or therapeutics that target pathways altered following bariatric surgery. The combinations can include therapeutics that impact homeostatic energy balance, including neuronal circuits, some originating in the hypothalamus and brain stem, as well as peripherally derived satiety, hunger and adiposity signals that modulate neural activity and regulate eating behavior. Dysregulation of one or more of these homeostatic components results in obesity. The hormonal signaling network that provides the brain input related to metabolic status and energy stores includes leptin, insulin, cholecystokinin (CCK), glucagon-like peptide-1 (GLP-1), peptide YY₃₋₃₆ (PYY₃₋₃₆), and ghrelin. Any of the peptides, proteins, and pathways detailed below can be targeted for the combination and/or combination rotational therapy described herein. The combinatorial therapies and rotational combinatorial therapies provided herein target a plurality thereof. Components of these pathways, including agonists and antagonists as appropriate, or down- or up-regulators of the pathways can be included in the combination and/or rotational combinatorial therapy to effect weight loss, or to treat or ameliorate comorbidities of overweight or obesity.

The following discussion provides and describes exemplary therapeutics that can be incorporated into a combinatorial weight loss protocol. The combinations can be rotated to produce rotational combinatorial protocols.

### Glucagon Like Peptide-1

The sequence of GLP-1 is set forth in SEQ ID NO:1, and the chemical formula is C₁₈₆H₂₇₅N₅₁O₅₉. The glucagon-like peptide-1 (GLP-1) is released by L-cells in response to food ingestion. Simulation of L-cells increases both GLP-1 and GLP-1 related peptides, such as glicentin, oxyntomodulin intervening peptide-2 and GLP-2. Agents described for use in the rotational combinatorial treatment herein can work by activating the Glucagon-like peptide-1 receptor (GLP-1R) or by inhibiting the breakdown of GLP-1 like dipeptidyl peptidase-4 (DPP-4) inhibitors (see Bruton, S., Int. J of Clin Pract. 68(5):557-567 (2014)). DPP-1 inhibitors and their uses are previously described (see e.g., WO 2002/068420, WO 2004/018467, WO 2004/018468, WO 2004/018469, WO 2004/041820, WO 2004/046148, WO 2005/051950, WO 2005/082906, WO 2005/063750, WO 2005/085246, WO 2006/027204, WO 2006/029769, WO2007/014886; WO 2004/050658, WO 2004/111051, WO 2005/058901, WO 2005/097798; WO 2006/068163, WO 2007/071738, WO 2008/017670; WO 2007/128721, WO 2007/128724, WO 2007/128761, or WO 2009/121945).

Glucagon-like peptide-1 receptor agonists can be formulated as therapeutics in the combinational therapy provided herein. In some examples, a GLP-1R agonist can be formulated in combination with additional receptor agonists (i.e., GIP and/or glucagon receptor agonist(s)). A single biagonist or triagonist peptide in the combinatorial rotational therapy herein can act as an agonist for multiple receptors of the peptides, proteins and/or pathways detailed above, or pathways involving hunger, digestion and/or metabolism. For example, a biagonist or triagonist peptide that acts as an agonist of one or more glucagon-like peptide-1 (GLP-1), glucose-dependent insulinotropic polypeptide (GIP) and glucagon receptors, such as the previously described triagonist peptide can be included in the combination therapy to effect weight loss (Finan et al., Nat Med 21(1):27-36 (2015)).

The peptides and corresponding SEQ ID NOs. are set forth below.

### Adiponectin

Adiponectin is a 244-amino-acid-long polypeptide (SEQ ID NO:2), protein hormone and adipokine, involved in regulating glucose levels as well as fatty acid breakdown (Iglesias, J. J., European Journal of Endocrinology 148 (3): 293-300). Adiponectin is secreted from adipose tissue into the bloodstream and is abundant in plasma relative to several other hormones. High adiponectin levels correlate with a lower risk of diabetes mellitus type 2 (Li et al., JAMA 302(2): 179-188 (2009)). Adiponectin plays a role in suppressing metabolic derangements that can result in type 2 diabetes, obesity, atherosclerosis, non-alcoholic fatty liver disease (NAFLD) and an independent risk factor for metabolic syndrome (Ukkola et al., J Mol Med 80 (11): 696-702 (2002); Iglesias, J. J., European Journal of Endocrinology 148 (3): 293-300). Adiponectin in combination with leptin has been shown to completely reverse insulin resistance in mice (Chen et al., Diabetologia 49 (6): 1292-302 (2006)). Adiponectin enhances insulin sensitivity primarily though regulation of fatty acid oxidation and suppression of hepatic glucose production (Li et al., JAMA 302 (2): 179 (2009)). Adiponectin exerts weight reduction effects via the brain, similar to the action of leptin; and adiponectin and leptin can act synergistically (Coppola et al., International Journal of Cardiology. 134 (3): 414-6 (2009)).

Adiponectin self-associates into larger structures. Initially, three adiponectin molecules bind together to form a homotrimer, and the trimers continue to self-associate and form hexamers or dodecamers. Studies showed that the high-molecular weight form of adiponectin is the most biologically active form regarding glucose homeostasis (Kuo et al., Int J of Obesity 35 (12): 1487-94 (2011)).

Plasma levels of adiponectin are lower in obese subjects than in lean subjects (Ukkola et al., J Mol Med 80 (11): 696-702 (2002)). Adiponectin is secreted into the bloodstream where it accounts for approximately 0.01% of all plasma protein at around 5-10 g/mL (mg/L). Studies show adiponectin is inversely correlated with body mass index in patient populations (Kuo et al. International Journal of Obesity. 35 (12): 1487-94 (2011)); a meta-analysis did not confirm this association in healthy adults (Renaldi et al., Acta Medica Indonesiana. 41 (1): 20-4 (2009)).

Weight reduction significantly increases circulating adiponectin concentrations (Ukkola et al., J Mol Med 80 (11): 696-702 (2002)). High-molecular-weight adiponectin is associated with a lower risk of diabetes with similar magnitude of association as total adiponectin (Kuo et al. International Journal of Obesity 35 (12): 1487-94 (2011)). Coronary artery disease is positively associated with high molecular weight adiponectin, but not with low molecular weight adiponectin (see Fisman et al., Cardiovascular Diabetology 13 (1): 103 (2014)).

### Leptin

Leptin is a 167 amino acid hormone (SEQ ID NO:3) predominantly made by adipose cells and enterocytes in the small intestine that helps to regulate energy balance by inhibiting hunger, which in turn diminishes fat storage in adipocytes. Leptin acts on cell receptors in the arcuate and ventromedial nuclei, as well as other parts of the hypothalamus and dopaminergic neurons of the ventral tegmental area, consequently mediating feeding (Brennan et al., Nature Clinical Practice. Endocrinology & Metabolism (6): 318-27 (2006); Bouret et al., Physiological Reviews 95 (1): 47-82 (2015); Elmquist et al., Neuron. 22 (2): 221-32 (1999)). Leptin regulates energy metabolism via activation of arcuate pro-opiomelanocortin (POMC) neurons in the arcuate nucleus of the hypothalamus. POMC neurons project to the paraventricular nucleus (PVN), where they induce satiety through activation of the brain MC4 receptors.

In the lateral hypothalamus, leptin inhibits hunger by 1) counteracting the effects of neuropeptide Y, a potent hunger promoter secreted by cells in the gut and in the hypothalamus, and 2) counteracting the effects of anandamide, another potent hunger promoter that binds to the same receptors as THC (Elias et al., Neuron. 23 (4): 775-86 (1999)). In the medial hypothalamus, leptin stimulates satiety by promoting the synthesis of α-MSH, a hunger suppressant (Fekete et al., J Neurosci 20 (4): 1550-8 (2000)).

Along with insulin (see, e.g., SEQ ID NO: 30) and amylin, leptin signals to the brain in proportion to the amount of fat that is stored in the body. Although leptin reduces appetite as a circulating signal, obese individuals generally exhibit a higher circulating concentration of leptin than normal weight individuals due to their higher percentage body fat (Considine et al., The New England Journal of Medicine 334 (5): 292-295 (1996)). Leptin also reduces appetite in response to feeding, but obese people develop a resistance to leptin, similar to resistance of insulin in type 2 diabetes patients, where elevated levels fail to control hunger and modulate weight in obese people. An important contributor to leptin resistance is changes to leptin receptor signaling, particularly in the arcuate nucleus, however, deficiency of, or major changes to, the leptin receptor itself are not thought to be a major cause. Triglycerides crossing the blood brain barrier (BBB) can induce leptin and insulin resistance in the hypothalamus (Forny-Germano et al., Front in Neurosci 12:1027 (2019)). Triglycerides can also impair leptin transport across the BBB. id.

Leptin interacts with amylin, a hormone involved in gastric emptying and creating a feeling of fullness. When leptin and amylin were given to obese, leptin-resistant rats, sustained weight loss occurred. A recombinant leptin analog, Metreleptin (see SEQ ID NO: 34), was approved by the US FDA in 2014 and the European Medication Association (EMA) in 2018 for the treatment of lipodystrophy (Müller et al., Nat Rev Drug Discov 21: 201-223 (2022)). While leptin supplementation is effective in individuals with genetic leptin deficiency, leptin supplementation has not been shown to be effective to lower body weight in obese individuals (Müller et al., Nat Rev Drug Discov 21: 201-223 (2022)). Combinations of small molecule leptin sensitizers withaferin A and celastrol with the hormones extendin 4 (see SEQ ID NO:21), FGF21 (see SEQ ID NO:22), or GLP1/glucagon improve leptin responsiveness in preclinical studies. Several MC4R agonists have been developed and LY2112688, MC4-NN-0453, MK-0493, and AZD2820, although clinical development has ceased (Müller et al., Nat Rev Drug Discov 21(3):201-223 (2021)). Due to its apparent ability to reverse leptin resistance, amylin has been suggested as possible therapy for obesity (Roth et al., PNAS 105 (20): 7257-62 (2008)). Leptin can be a target for developing a therapeutic, in combination with any of the peptides or therapeutics described herein, or known to be involved with metabolic pathways, in accord with a rotational regimen, such as, for example, to mimic satiety.

### Oxyntomodulin

Oxyntomodulin is a 37-amino acid peptide (SEQ ID NO:4) hormone produced by the oxyntic (fundic) cells of the oxyntic (fundic) mucosa in the colon.

Oxyntomodulin suppresses appetite; the mechanism by which it does so is not understood. It binds to the GLP-1 receptor and to the glucagon receptor, but it is not known whether the effects of the hormone are mediated through these receptors or through an unidentified receptor.

Oxyntomodulin has been investigated as a blood-glucose regulating agent in connection with diabetes. Oxyntomodulin is a candidate for treating obesity because of its ability to suppress appetite (Shankar et al., Diabetes. 67 (6): 1105-1112 (2018)). In a 4-week study, healthy overweight and obese subjects treated with oxyntomodulin injections had an average weight loss of 2.3±0.4 kg compared to those treated with saline who had an average of 0.5±0.5 kg (Wynne et al., Diabetes. 54 (8): 2390-2395 (2005)).

### Sermorelin Acetate

Sermorelin acetate (GHRH (1-29)) is peptide analog of growth hormone-releasing hormone (GHRH; see SEQ ID NO:29) which is used as a diagnostic agent to assess growth hormone (GH) secretion to diagnose growth hormone deficiency. Sermorelin acetate is a 29-amino acid polypeptide representing the 1-29 fragment from endogenous human GHRH, thought to be the shortest fully functional fragment of GHRH (Prakash et al., BioDrugs 12(2):139-57 (1999); Rang, Dale, Ritter & Moore. Pharmacology. Edinburgh, Churchill Livingstone (2005)).

Studies show that longer term treatment (i.e., 5 months) with sermorelin results in increased GH and IGF-1 and increased lean body mass that were not observed with shorter treatment (2-6 weeks) (Corpas et al., J Clin Endocrinol Metab (1992) 75:530-5; Vittone et al., Metabolism (1997) 46:89-96; Khorram et al., J Clin Endocrinol Metab (1997) 82:1472-9).

### Peptide YY (PYY)

Peptide YY (PYY) is a short (36-amino acid) peptide released from cells in the ileum and colon in response to feeding. In the blood, gut, and other elements of periphery, PYY acts to reduce appetite; similarly, when injected directly into the central nervous system, PYY is also anorexigenic, i.e., it reduces appetite (Woods et al., J Clin Endocrinol Metab. 93 (11 Suppl 1): S37-50 (2008)).

The two major forms of peptide YY (PYY) are PYY₁₋₃₆ and PYY₃₋₃₆ (SEQ ID NO:37), which have PP fold structural motifs. PYY₁₋₃₆ is rapidly cleaved by DPP-IV to its major active form, Peptide YY₃₋₃₆. PYY₃₋₃₆ is a linear polypeptide that contains 34 amino acids with structural homology to NPY and pancreatic polypeptide (Murphy et al., Nature 444 (7121): 854-59 (2006)). PYY₃₋₃₆ is the most common form of circulating PYY, which binds to the NPY receptor type 2 (Y2R). This receptor is highly expressed in parasympathetic and sympathetic neurons of the periphery as well as in several regions of the CNS, including the limbic and cortical areas and the brainstem (Stadlbauer et al., Neuroendocrinol. 38: 1-11 (2015)).

Oxyntomodulin and peptide tyrosine-tyrosine (PYY) are released from intestinal enteroendocrine cells following a meal. These circulating hormones are considered to be satiety signals, as they decrease food intake, body weight, and adiposity in rodents (Wynne et al., Nat Cin Pract Endocrinol Metab. 2(11), 612-20 (2006)).

While some studies have shown that obese persons have lower circulating levels of PYY postprandially, other studies have reported that they have normal sensitivity to the anorectic effect of PYY₃₋₃₆. It is unknown whether reduction in PYY sensitivity contributes to obesity rather than the reduction of leptin sensitivity.

The anorectic effect of PYY is a candidate for a future obesity drug (Murphy et al., Nature (2006) 444 (7121): 854-59). Attempts to use PYY directly as a weight-loss drug have met with some success (Bartolomé et al., Obes Surg. (2002) 12(3):324-7). PYY decreases food intake and body weight in rodents and humans (Batterham et al., Nature (2002) 418: 650-654; Batterham et al., N. Engl. J. Med. (2003) 349:941-948). Researchers noted the caloric intake during a buffet lunch offered two hours after the infusion of PYY was decreased by 30% in obese subjects (P<0.001) and 31% in lean subjects (P<0.001) (Batterham et al., The New England Journal of Medicine (2003) 349 (10):941-8). Several PYY analogues have been developed and have entered clinical trials for weight loss treatment including NN9748, NNC0165-1875, and NNC0165-1875+semaglutide (Poulsen, et al., Pharm. Res. (2021) 8: 1369-1385).

Protein consumption boosts PYY levels, so some benefit was observed in experimental subjects in reducing hunger and promoting weight loss (Batterham et al., Cell Metabolism. 4 (3): 223-233 (2006)). This can partially explain the weight-loss experienced with high-protein diets, but the high thermic effect of protein appears to be the leading cause.

Obese patients undergoing gastric bypass showed marked metabolic adaptations, resulting in frequent diabetes remission 1 year later. When the confounding of calorie restriction is factored out, β-cell function improves rapidly, very possibly under the influence of enhanced GLP-1 responsiveness. Insulin sensitivity improves in proportion to weight loss, with a possible involvement of PYY (Nannipieri et al., J. Clin. Endocrinol. Metab. 98 (11): 4391-9 (2013)).

### Amylin

The Amylin peptide hormone is co-secreted with insulin from the pancreatic β-cells at a ratio of approximately 100:1 (insulin:amylin) into the blood circulation and is cleared by peptidases in the kidney, so it does not occur in the urine (Higham et al., Eur. J. Biochem. 267 (16): 4998-5004 (2000)). Amylin functions as part of the endocrine pancreas and plays a role in glycemic regulation by slowing gastric emptying and promoting satiety, thereby preventing post-prandial spikes in blood glucose levels.

Amylin's metabolic function is well-characterized as an inhibitor of the appearance of nutrients [especially glucose] in the plasma (Pittner et al., J. Cell. Biochem 55 Suppl: 19-28 (1994)). The overall effect is to slow the rate of appearance (Ra) of glucose in the blood after eating; this is accomplished via coordinate slowing down gastric emptying, inhibition of digestive secretion [gastric acid, pancreatic enzymes, and bile ejection], and a resulting reduction in food intake. Appearance of new glucose in the blood is reduced by inhibiting secretion of the gluconeogenic hormone glucagon. These actions, which are mostly carried out via a glucose-sensitive part of the brain stem, the area postrema, can be over-ridden during hypoglycemia. They collectively reduce the total insulin demand (Ratner et al., Diabetic Medicine 21 (11): 1204-12 (2004)).

The clinical application of native amylin in treating obesity is hampered by aggregation and pancreatic islet death (Ling et al., Curr. Protein Pept. Sci. 20, 944-957 (2019)). Several Amylin peptide analogs have been developed and include Pramlintide (see SEQ ID NO:38), Cagrilintide (SEQ ID NO:43), and ZP 8396. A 2008 study reported a synergistic effect of a human leptin (metrelepin) and an amylin analog (pramlintide) for obesity treatment in diet-induced obese rats and human subjects by restoring hypothalamic sensitivity to leptin (Roth et al., PNAS 105(20):7257-62 (2008)). Pramlintide is approved for patients with Type 1 and Type 2 diabetes in combination with insulin, metformin, or sulfonylurea. Cagrilintide is a long-acting amylin analogue amenable for once-weekly dosing. Dual-acting amylin and calcitonin receptor agonists can induce weight loss in pre-clinical models of obesity (Müller et al., Nat Rev Drug Discov 21: 201-223 (2022)). Dual agonists include davalintide (AC2307; SEQ ID NO:27), KBP-088 (see SEQ ID NO:31), KBP-089 (see SEQ ID NO:32), and KBP-042 (SEQ ID NO:42).

### Tesamorelin

Tesamorelin is a 44 amino acid synthetic form of growth-hormone-releasing hormone (GHRH) which is used in the treatment of HIV-associated lipodystrophy, approved initially in 2010. It is produced and developed by Theratechnologies, Inc. of Canada. Tesamorelin is sold under the trademark Egrifta® medication.

Released GH binds with receptors present on various body organs and regulates body composition; the regulation is primarily due to the combination of anabolic and lipolytic mechanisms. The main mechanisms by which tesamorelin reduces body fat mass are lipolysis followed by reduction in triglyceride levels (Benedini et al., BioDrugs. 22 (2): 101-12 (2008)).

### Pancreatic Polypeptide

Pancreatic polypeptide (PP) is a 36 amino acids long peptide (human sequence of mature PP set forth in SEQ ID NO:40 (APLEPVYPGD NATPEQMAQY AADLRRYINM LTRPRY) that regulates pancreatic secretion activities by both endocrine and exocrine tissues. PP is synthesized as a 95 aa polypeptide precursor in the pancreatic islets of Langerhans. PP also affects hepatic glycogen levels and gastrointestinal secretions. PP secretion in humans increases after a protein meal, fasting, exercise, and acute hypoglycemia, and decreases after increased food intake and in patients with anorexia nervosa. On fasting, pancreatic polypeptide concentration is 80 pg/mL; and after meal consumption the levels rise to 640 to 800 pg/mL.

Glucose and fats also induce PP and increase the PP level; however, upon parenteral introduction, the hormone levels do not change. Peripheral administration of PP has been shown to decrease food intake in rodents (Batterham et al., The Journal of Clinical Endocrinology and Metabolism. 88 (8): 3989-92 (2003)). PP inhibits pancreatic secretion of fluid, bicarbonate, and digestive enzymes; stimulates gastric acid secretion; is the antagonist of cholecystokinin; and opposes pancreatic secretion stimulated by cholecystokinin (Washabau, Canine and Feline Gastroenterology. Philadelphia, PA, Saunders (2013)).

### Gastric Inhibitory Polypeptide

Gastric Inhibitory Polypeptide (GIP2, herein) is an endogenous 42-amino acid peptide synthesized by K cells, which are found in the mucosa of the duodenum and the jejunum of the gastrointestinal tract (Costanzo, Physiology. Philadelphia, PA, Saunders/Elsevier (2014)). This endogenous hormone known to be upregulated post Rou-en-Y gastric bypass. GIP is thought to induce insulin secretion, which is stimulated primarily by hyperosmolarity of glucose in the duodenum which has led to referencing GIP as glucose-dependent insulinotropic peptide, while retaining the acronym “GIP” (Thorens, Diabète & Métabolisme. 21 (5): 311-8 (2014)). In addition to its role as an incretin, GIP is known to inhibit apoptosis of the pancreatic beta cells and promote their proliferation.

### Enterostatin

Enterostatin is a peptide previously shown to selectively reduce fat intake, lower cholesterol, reduce angiogenesis, and regulate analgesia. Enterostatin is produced in the gastric mucosa and epithelium of the small intestine, is absorbed from the digestive tract, and functions as a strong anorectic peptide to selectively decrease fat consumption in rodents (D. L. Nelson, D. R. Gehlert, in Comprehensive Medicinal Chemistry II, 2007). Enterostatin interacts with a variety of hormone systems and receptors (Charlotte Erlanson-Albertsson, in Handbook of Biologically Active Peptides (Second Edition), 2013). Enterostatin knock-out mice showed significantly increased serum cholesterol compared to wild-type controls and administration of enterostatin and its peptide fragment DPR reduce serum cholesterol after oral administration in mice (Miller et al., Am J Physiol Endocrinol Metab. 297(4):E856-65 (2009); Takenaka et al., Biosci Biotechol Biochem. 67(7):1620-22 (2003)).

Three enterostatin protein sequences have been identified in rats and humans, designated VPDPR (rat and human) (see SEQ ID NO:20), APGPR (human; SEQ ID NO:18), and VPGPR (rat; SEQ ID NO:19), and have been shown to reduce food intake after chronic ingestion of a high-fat diet in preclinical animal models. In a phase II randomized placebo-controlled trail of 18 obese patients, intravenous enterostatin administration did not impact feelings of hunger, satiety, or food preference.

### Ghrelin

Ghrelin (SEQ ID NO:23) is a stomach-derived peptide hormone that signals to the hypothalamus to stimulate homeostatic food intake and hunger; ghrelin levels increase before a meal and when a person is hungry, and levels decrease after a meal. Daily administration of ghrelin caused weight gain by reducing fat utilization in mice and rats. Strategies to target Ghrelin include suppressing levels of the hormone or antagonizing its receptor, growth hormone secretagogue receptor (GHSR) (Müller et al., Nat Rev Drug Discov 21: 201-223 (2022)). GHSR antagonists include antimicrobial peptide 2 (LEAP2). Approaches to decrease circulating Ghrelin include the peptide vaccine CYT009-GhrQb, the peptide-binding compounds Nox-B11, and the ghrelin analog AZP-531 (SEQ ID NO:15).

Following sleeve gastrectomy, ghrelin levels decreased and remained low for at least 5 years following surgery (Bohdjalian et al., Obes Surg 20:535-540 (2010)). Fasting ghrelin also decreased after laparoscopic sleeve gastrectomy and postsurgical circulating ghrelin decreased following Roux-en-Y gastric bypass, compared to both obese and normal weight control subjects.

The role of ghrelin in satiety and hunger and the changes in ghrelin regulation and expression following weight loss surgery and between normal and overweight individuals indicate the ghrelin pathway can be a target for weight loss and for inclusion in the rotational combinatorial therapy described herein.

### Cholecystokinin (CCK)

Multiple isoforms of CCK are released after food consumption. CCK mediates satiety by acting on CCK receptors distributed throughout the central nervous system. In response to meal initiation, plasma CCK levels have been reported to rise within 15 minutes (Liddle et al., J Clin Invest. 75(4): 1144-1152 (1985)). CCK also has stimulatory effects on the vagus nerve, which can be inhibited by capsaicin (Holzer et al., American Journal of Physiology. Gastrointestinal and Liver Physiology 275 (1): G8-G13 (1998)). The stimulatory effects of CCK oppose those of ghrelin, which has been shown to inhibit the vagus nerve (Kobelt et al., American Journal of Physiology 288 (3): R751-8 (2005)).

CCK was the first gut hormone reported to affect appetite and has been shown to dose-dependently reduce food intake in both rats and humans (Gibbs et al., Nature. 245: 323-325 (1973); Lieverse et al., Gut. 36(2): 176-179 (1995)). The effects of CCK vary between individuals, and are demonstrated in animal studies. For example, in rats, CCK administration significantly reduces hunger in adult males, but is less effective in younger subjects, and less effective in females. The hunger-suppressive effects of CCK also are reduced in obese rats (Fink et al., Experimental Brain Research 123 (1-2): 77-83 (1998)).

Within the GI tract, CCK is predominantly synthesized and released from the duodenum and jejunum, where its local regulatory effects include stimulation of gallbladder contraction and inhibition of gastric emptying (Buffa et al., Gastroenterology. 70(4): 528-532 (1967; Dufresne et al., Physiol Rev. 86(3): 805-847 (2006)). The mechanism for hunger suppression by CCK is thought to be related to a decrease in the rate of gastric emptying (Shillabeer et al., The American Journal of Physiology 252(2): R353-R360 (1987)).

The role of CCK in appetite and food intake and hunger suppression by CCK indicate CCK can be a target for weight loss and for inclusion in the rotational combinatorial therapy described herein.

### Vasoactive Intestinal Peptide

Vasoactive intestinal peptide (VIP) is an endogenous peptide hormone that is 28 amino acid residues. In the digestive system, VIP was shown to induce smooth muscle relaxation (lower esophageal sphincter, stomach, gallbladder), stimulate secretion of water into pancreatic juice and bile, and cause inhibition of gastric acid secretion and absorption from the intestinal lumen (Bowen et al., “Vasoactive Intestinal Peptide,” Pathophysiology of the Endocrine System: Gastrointestinal Hormones. Colorado State University). VIP stimulates intestinal secretion of water and electrolytes and relaxation of enteric smooth muscle, dilating peripheral blood vessels, stimulating pancreatic bicarbonate secretion, and inhibiting gastrin-stimulated gastric acid secretion (“Vasoactive intestinal polypeptide”. General Practice Notebook. Retrieved 2009-02-06). These effects work together to increase motility (Bergman et al., Atlas of Microscopic Anatomy: Section 6—Nervous Tissue. URL:.anatomyatlases.org).

VIP can be a target for weight loss and for inclusion in the rotational combinatorial therapy described herein.

### Glicentin

Glicentin is a 69 amino acid peptide secreted after the processing of proglucagon along with the following peptides listed below:
Pre Proprotein (Proglucagon Preproprotein) (SEQ ID NO:13)
Signal peptide (1-20; SEQ ID NO:39)—removed from preproglucagon to form proglucagon
Glicentin (21-89) (Glicentin 21-29 is set forth in SEQ ID NO:24)
Glicentin-related pancreatic polypeptide (GRPP, 21-50; set forth in SEQ ID NO:25)
Oxyntomodulin (OXY or OXM, 53-89; set forth in SEQ ID NO:4)
Glucagon (53-81; set forth in SEQ ID NO:27)
Glucagon-like peptide 1 (GLP-1, 92-128 set forth in SEQ ID NO:1)—first six residues further cleaved
Glucagon-like peptide 2 (GLP-2, 146-178; set forth in SEQ ID NO:28)
   Thus, Glicentin contains the entire sequence of glucagon and glicentin-related pancreatic polypeptide. There is no known glicentin receptor, so the mechanism of action is not well characterized. However, the concentration of glicentin in plasma is altered in patients experiencing diabetes and obesity, and following foregut surgery. Glucagon regulates systems metabolism including regulating acute and chronic thermogenic affects. (Kleinert et al., Int J Mol Sci. 20(21): 5407 (2019)).

### GDF15

Macrophage inhibitory cytokine 1 (MIC1; also known as GDF15; SEQ ID NO:33) reduces body weight through appetite suppression. Exogenous administration of recombinant GDF15 or GDF15 analogues decreases body weight in diet-induced obese mice and non-human primates (Mullican, S. E. et al., Nat. Med. 23, 1150-1157 (2017)). GDF15 agents in phase 1 clinical studies include LA-GDF15 and the GDF15 agonists LY-3463251 and JNJ-9090/CIN-109.

### Summary

Any of the peptides listed above, and other peptides and therapeutics that are known or thought to impact (i.e., accelerate or initiate) weight loss or that are associated with a pathway involving hunger or digestion or metabolism can be administered in combinations and/or in accord with the rotational protocol set forth herein. In some examples the peptides can be a synthesized peptide that can act as an agonist (or antagonist depending upon the pathway) for a receptor or receptors of any of the peptides and proteins and pathways detailed above, or pathways involving hunger, digestion and/or metabolism. In some examples, a single peptide in the combination and combinatorial rotational therapies provided herein can act as an agonist for multiple receptors of the peptides, proteins and/or pathways detailed above, or pathways involving hunger, digestion and/or metabolism. A combination of any of peptides and/or other therapeutics administered in combination and/or in accord with a rotational regimen, increases the efficacy of weight loss compared to administration of monotherapy or administration of a combination without the rotational component.

a. Therapeutic Combinations and Regimens

This section details how protocols provided herein have been developed and successfully employed for obesity, as an exemplary disease, disorder, or condition, and the working examples include protocols for treatment of overweight and obesity and other diseases, disorders, and conditions.

The protocols described herein and below are based on research that shows changes in hormonal secretions after bariatric surgery (see e.g., Ionut et al., (2015) Obesity 21(6):1093-1103). The gut-brain axis is a major component of appetite regulation. The gut hormones have either anorexic or orexigenic actions on food intake and it is now evident that these gut hormone secretions are altered following bariatric surgery. Some of the key peptides for administering in the combinatorial and/or rotational combinatorial therapy described herein, and their basal hormone levels and levels after bariatric surgery as shown in previous studies, are listed in Table 10, below, and are shown in FIG. 2 (see also Tak et al., Curr Obesity Reports (2021) 10:14-30), with reference to gastro-neuropathology and with their physiologic influences on the gut-brain.

TABLE 10
Exemplary Peptides for Administration for Weight Loss

| | | | Hormone |
|---|---|---|---|
| | | | Level After |
| | | Basal Hormone | Meal |
| peptide | Study Design | Level | stimulus |
| Ghrelin | Cross sectional | Suppressed | Not tested |
| | Prospective | 14% reduced at 6 weeks | Suppressed |
| | Prospective | No change at 18 months | Not tested |
| | Prospective | 44% increased at 6 months and | Not tested |
| | | 62% increase at 12 months | |
| | Cross-sectional | Reduced compared to obese and | No changes |
| | | normal weight controls | |
| | Cross-sectional | 45% lower than obese controls | Suppressed |
| | Prospective | Reduced at 12 months | Not tested |
| | Prospective | 64% increase to 6 months. | Not tested |
| Peptide YY | Cross-sectional | No change | Increased |
| (PYY) | 12 months postop | No change | Increased |
| | 3 years postop | No change | Increased |
| | Prospective | No change | Increased |
| | Prospective | No change | Increased |
| Glucagon-like | 12 months postop | No change | Increased |
| peptide 1 | Preop in 6 months postop | No change | Increased |
| (GLP-1) | Preop in 3 weeks postop | No change | Increased |
| Glucagon | Preop in 3 weeks postop | Reduced and type 2 diabetes | Not tested |
| dependent | | | |
| insulinotropic | | | |
| polypeptide | | | |
| (GIP) | | | |
| Cholecystokinin | Preop and 6 months postop | No change | Not tested |
| (CCK) | Preop in 6 months postop | No change | No change |
| Pancreatic | 12 months postop | No change | No change |
| polypeptide | Preop + 6-9 months postop | No change | Suppressed |
| (PP) | | | |

The data presented herein demonstrate an integrated model for the roles of these gut/adipose/brain peptides and others to regulate appetite in a synchronous fashion, similar to the suppression demonstrated in surgical gastric bypass patients. Due to downregulation of receptors and tolerance that develops almost inevitably with a pharmacotherapeutic agent, regimens provided herein not only include combinations of therapeutics, but also can include a rotation of differing therapeutics, such as, for example, therapeutic peptides, such as rotating a peptide or combinations of peptides every 3 months. In other examples, the combination therapy can be rotated every 1, 2, 3, 4, 5, 6, 7, 8, 9, 10, 11, 12, 13, 14, 15, 16, 17, 18, 19, 20, 21, 22, 23, 24 weeks or longer, such as every 3, 6, 9, 12, 15, 18, 21, 24, 27, 30, 33, 36 months, or more. In some examples, the regimen can include rotation of different peptides every three months. This rotation will continue to drive weight loss as different receptors even among peptides within the same families will be activated.

b. Combination Drug Therapy to Mimic Gastric Bypass

Various hormone levels increase following bariatric surgery, are thought to contribute to the weight loss following bariatric surgery, and the modified levels persist and are long lasting. The combination therapy described herein can be designed to mimic the hormonal and other signaling changes that occur after bariatric surgery, such as Roux-en-Y (Ochner et al., Int J Obes 35(2):153-166 (2011)). The table below includes targets/pathways for targeting for weight loss, exemplary medications for targeting (i.e., activating or inhibiting) the pathways, the function of the administered medication, and exemplary references detailing the target pathways and/or medications for use for targeting the pathways. Exemplary combinations for mimicking changes that occur following gastric bypass include exemplary medications for 2 or more of the targets listed in the table below:

| Target | Exemplary Medications | Function | Exemplary Reference(s) |
|---|---|---|---|
| PYY | PYY peptide; PYY agonist; | Elevate PYY or | Karra et al., J Physiol. 2009 |
| | PYY-receptor agonist (e.g., | activate PYY-R(s) | Jan 1; 587(Pt 1): 19-25; |
| | hypothalamic neuropeptide Y2 | | Chelikani et. al., Am J |
| | receptor (NPYR2) or NPYR1 | | Physiol Regul Integr Comp |
| | agonist) | | Physiol. (2007) 293: R39- |
| | | | R46; Chelikani et al., Am J |
| | | | Physiol Regul Integr Comp |
| | | | Physiol. (2007) 293: R39- |
| | | | R46; Koegler et al., Diabetes |
| | | | (2005) 54: 3198-3204. |
| GLP-1 | Dulaglutide (Trulicity) | Elevate GLP-1 or | Laferrère, B. Diabetes and |
| | (weekly) | activate GLP-1R | Metaboism (2009) 35(2): 513- |
| | Exenatide extended release | (GLP-1R agonist) | 517; |
| | (Bydureon bcise) (weekly) | | |
| | Exenatide (Byetta) (2x daily) | | |
| | Semaglutide (Ozempic) | | |
| | (weekly) | | |
| | Liraglutide (Victoza, Saxenda) | | |
| | (daily) | | |
| | Lixisenatide (Adlyxin) (daily) | | |
| | Semaglutide (Rybelsus) (by | | |
| | mouth 1x day) | | |
| | Tirzepatide (GLP-1/GIP dual | | |
| | agonist) | | |
| GIP | Tirzepatide (GLP-1/GIP dual | Elevate GIP or | Laferrère, B. Diabetes and |
| | agonist) | activate GIP-R | Metabolism (2009) |
| | | (GIPR agonist) | 35(2): 513-517; Mroz et al., |
| | | | Mol. Metab. 20, 51-62 |
| | | | (2019); Zhang et al., Cell |
| | | | Metab. 33: 833-844.e5 (2021) |
| Cholecystokinin | CCK peptide | Elevate CCK | Mumphrey et al., |
| (CCK) | | | Neurogastroenterol Motil |
| | | | (2013) 25(1): e70-e79; |
| | | | Peterli et al. (2012) Obes. |
| | | | Surg., 22 (5): |
| | | | 740-748 |
| Pancreatic | PP; PP analogs; synthetic PP | Elevate PP; | |
| polypeptide (PP) | | decrease PP | |
| | | degradation or | |
| | | inactivation | |
| adiponectin | Adiponectin peptide | | Unamuno et al., Nutrients |
| | | | (2019) 11(9): 2069); Vendrell |
| | | | et al. (2004) Obes |
| | | | Res. 12: 962- |
| | | | 971; Garcia de la Torre et al. |
| | | | (2008) J Clin Endocrinol |
| | | | Metab. 93: 4276-4281; Faraj |
| | | | et al., (2003) J Clin |
| | | | Endocrinol Metab. 88: 1594- |
| | | | 1602; Kotidis et al., (2006) |
| | | | Obes Surg. 16: 1425-1432. |
| leptin | Leptin | increase leptin | Garcia de la Torre et al. |
| | | levels | (2008) J Clin Endocrinol |
| | | | Metab. 93: 4276-4281; |
| | | | Faraj et al., (2003) J Clin |
| | | | Endocrinol Metab. |
| | | | 88: 1594-1602; Kotidis et |
| | | | al., (2006) Obes Surg. |
| | | | 16: 1425-1432. |
| oxyntomodulin | Oxyntomodulin peptide | Increase | Borg et al., (2006) Br J |
| | | oxyntomodulin | Surg. 93: 210-215; Kellum |
| | | levels | et al., (1990) Ann Surg. |
| | | | 211: 763-770. discussion |
| | | | 770-1; Barry et al., (1977) |
| | | | Am J Clin Nut 30: 32-42; |
| | | | Holdst et al., (1979) Scand |
| | | | J Gastroenterol 14: 205- |
| | | | 207 |
| Amylin | long-acting amylin analog | Increase amylin | Larsen et al., (2022) |
| | (cagrilintide, AM833, or | | “Does receptor balance |
| | NNC0174-0833); Amylin | | matter? -- comparing the |
| | agonists; amylin peptides | | efficacies of the dual |
| | | | amylin and calcitonin |
| | | | receptor agonists |
| | | | cagrilintide and KBP-336 |
| | | | on metabolic parameters |
| | | | in preclinical models,” |
| | | | Biomedicine & |
| | | | Pharmacotherapy |
| | | | 156: 113842. |
| Glicentin | Glicentin peptide | Increase glicentin | Nielsen et al., (2020) J |
| | | | Clin Endocrinol Metab |
| | | | 105(4): dgaa061; Poitou et |
| | | | al., (2018) Surgery for |
| | | | Obesity and Related |
| | | | Diseases 14(7): 929-935; |
| | | | Raffort et al., (2017) |
| | | | Obesity Surgery 27: 1581- |
| | | | 1588 |

Additional medications targeting pathways modified after bariatric weight loss surgery also can be added to the combinatorial regimen described herein. Such medications include, for example, those that are not used because they are ineffective as a monotherapy, but as described herein can be effective, or efficacy can be increased compared to monotherapy, when administered in combination with other medications, for example in a combinatorial therapy, or a combinatorial rotational therapy. For example, ghrelin receptor antagonists, medications that decrease circulating ghrelin, anti-ghrelin vaccines (e.g., the peptide vaccine CYT009-GhrQb), ghrelin receptor inverse agonists (GHSR-IA), the peptide-binding compounds (e.g., Nox-B11), and any other medication(s) or therapy that decrease ghrelin or ghrelin-associated activation pathways can be included in the combination therapy herein. In other examples, receptor agonists that mimic the action of peptides that are increased after bariatric surgery can be included (i.e., PYY, CCK, and/or PP receptor agonists). In some examples, drugs for inclusion in the combination therapy herein also can include neuropeptide Y (NPY) antagonists, melanocortin-4 receptor (MC4R) agonists, cannabinoid-1 receptor antagonists, and agonists and antagonists as appropriate for increasing weight loss and/or decreasing the comorbidities of overweight and/or obesity.

### G. PHARMACEUTICAL PRODUCTION, COMPOSITIONS, AND FORMULATIONS

### 1. Formulation and Administration of the Combinatorial Therapy

The combinations containing the therapeutics for the combination and/or rotational combinatorial therapy described herein can be formulated as pharmaceutical compositions provided for administration by a desired route, such as oral, mucosal, intravenous, and others. Pharmaceutically acceptable compositions are prepared in view of approvals for a regulatory agency or other agency prepared in accordance with generally recognized pharmacopeia for use in animals and in humans, and also, for agricultural applications, for plants. Typically, compounds are formulated into pharmaceutical compositions using techniques and procedures well-known in the art (see e.g., Ansel Introduction to Pharmaceutical Dosage Forms, Fourth Edition, 1985, 126).

The primary advantages of oral drug delivery compared with injection or nasal spray administration are greater acceptability, ease of administration and convenience for patients. With the protocol described herein, oral delivery of a combination of medications (i.e., therapeutic peptides) would increase compliance over frequent subcutaneous injections.

Oral delivery systems can pose a challenge due to proteolytic cleavage in the digestive tract, limited intestinal uptake of the intact molecule, and absorption of peptides that can be hindered by their physical characteristics, including the polarity and size of the dosage form, and their susceptibility to aggregation or degradation by the local pH in the tract.

To overcome the barriers mentioned above, various strategies have been adopted to increase bioavailability of proteins and peptides. Different approaches can be used to enhance systemic exposure of peptides following oral delivery of the combination therapy described herein. In some examples, one or more of the following mechanisms can be utilized to enhance delivery of the combination of therapeutics described: 1) permeation enhancers with added glycosylation, PEGylation or lipidation to increase uptake across cell membranes; 2) enzyme inhibitors such as, for example, carnitine esters, soybean trypsin inhibitors, deoxycholic acids and organic acids to prevent degradation in the gut; 3) modulation of protein to prevent degradation, such as, for example, by adding stabilizing side chains to reduce degradation; 4) additional of particulate systems, polymeric micro and nanoparticles, liposomes, micro-emulsification or polymeric micelles; 5) addition of multifunctional polymers, to include, for example, Poly(alkycyanacrylate) chitosan to assist with mucosal adhesion and bypass tight junctions; and 6) modification of ligand-specific binding and uptake, to include binding to B12, biotin, lecithin, and folate.

The therapeutic molecules in the combinations can be administered in forms that increase half-life. For example, the therapeutic molecules in the combinations can be provided as part of a liposome or multicellular laminar vesicle or other such delivery vehicle.

The therapeutic molecule provided in the combinations herein can be modified by a polymer prior to administration. In some examples, the polymer is a polyalkylene glycol, dextran, pullulan or cellulose. Polyalkylene glycol polymers, which can modify the therapeutic molecule include polyethylene glycol (PEG) and methoxypolyethylene glycol (mPEG). In examples where the therapeutic molecule is modified by PEG, the PEG can by branched or linear. In some embodiments, the polymer can be produced by reaction with methoxy-poly(ethylene glycol)-succinimidyl butanoate (mPEG-SBA) (5 kDa); methoxy-poly(ethylene glycol)-succinimidyl butanoate (mPEG-SBA) (20 kDa); methoxy-poly(ethylene glycol)-succinimidyl butanoate (mPEG-SBA) (30 kDa); methoxy-poly(ethylene glycol)-succinimidyl α-methylbutanoate (mPEG-SMB) (20 kDa); methoxy-poly(ethylene glycol)-succinimidyl α-methylbutanoate (mPEG-SMB) (30 kDa); methoxy-poly(ethylene glycol)-butyraldehyde (mPEG-butyraldehyde) (30 kDa), methoxy-poly(ethylene glycol)-succinimidyl propionate (mPEG-SPA) (20 kDa); methoxy-poly(ethylene glycol)-succinimidyl propionate (mPEG-SPA) (30 kDa); (methoxy-poly(ethylene glycol))₂-N-hydroxysuccinimide ester (mPEG₂-NHS) (10 kDa branched); (methoxy-poly(ethylene glycol))₂-N-hydroxysuccinimide ester (mPEG₂-NHS) (20 kDa branched); (methoxy-poly(ethylene glycol))₂-N-hydroxysuccinimide ester (mPEG₂-NHS) (40 kDa branched); (methoxy-poly(ethylene glycol))₂-N-hydroxysuccinimide ester (mPEG₂-NHS) (60 kDa branched); biotin-poly(ethylene glycol)-N-hydroxysuccinimide ester (biotin-PEG-NHS) (5 kDa biotinylated); poly(ethylene glycol)-p-nitrophenyl carbonate (PEG-p-nitrophenyl-carbonate) (30 kDa); or poly(ethylene glycol)-propionaldehyde (PEG-propionaldehyde) (30 kDa).

Any of the above approaches can be used to formulate the combination(s) of therapeutics described herein.

The combinations can be used for therapeutic, prophylactic, cosmetic, and/or diagnostic applications. The combinations containing the therapeutics for the rotational combinatorial therapy described herein can be formulated with a pharmaceutical acceptable carrier or diluent. Generally, such pharmaceutical combinations of compositions include components that do not significantly impair the biological properties or other properties of the cargo.

Each component in the combination is pharmaceutically and physiologically acceptable so that it is compatible with the other ingredients and not injurious to the subject to whom it is to be administered.

The formulations can be provided in unit dosage form and can be prepared by methods well-known in the art of pharmacy, including but not limited to, tablets, pills, powders, granules, sterile parenteral solutions or suspensions, oral solutions or suspensions, oil water emulsions, liquid solutions or suspensions (e.g., including injectable, ingestible and topical formulations (e.g., eye drops, gels, pastes, creams, or ointments)), aerosols (e.g., nasal sprays, and inhalers), liposomes, suppositories, pessaries, injectable and infusible solution and sustained release forms. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY. Each unit dose contains a predetermined quantity of therapeutically active compound or compounds sufficient to produce the desired therapeutic effect, in association with the required pharmaceutical carrier, vehicle or diluent. Examples of unit dose forms include ampoules. When administered systemically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well-known or will be apparent to those skilled in the art and are described in more detail in, e.g., “Remington: The Science and Practice of Pharmacy (Formerly Remington's Pharmaceutical Sciences)”, 19th ed., Mack Publishing Company, Easton, Pa. (1995).

Pharmaceutical combinations provided herein can be in various forms, e.g., in solid, semi-solid, liquid, powder, aqueous, and lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, and powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20th Edition. Baltimore, MD: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

The therapeutics for the rotational combinatorial therapy described herein can be prepared in a mixture with a pharmaceutically acceptable carrier. Techniques for formulation and administration of the compounds are known to one of skill in the art (see e.g., “Remington's Pharmaceutical Sciences,” Mack Publishing Co., Easton, Pa.). This therapeutic composition can be administered intravenously or through the nose or lung, such as a liquid or powder aerosol (lyophilized). The composition also can be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

### 2. Dosage Forms

Therapeutic formulations can be administered in many conventional dosage formulations. Dosage formulations of therapeutics for the rotational combinatorial therapy described herein can be prepared for storage or administration by mixing the compound having the desired degree of purity with physiologically acceptable carriers, excipients, or stabilizers. Such materials are non-toxic to the recipients at the dosages and concentrations employed, and can include buffers such as Tris HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN (polysorbates), Pluronic, polyethylene glycol, and others.

When used for in vivo administration (i.e., to patients), the formulation should be sterile and can be formulated according to conventional pharmaceutical practice. This is readily accomplished by filtration through sterile filtration membranes, prior to or following lyophilization and reconstitution. For example, a therapeutic peptide for a rotational combination therapy described herein can be provided as lyophilized powder that is reconstituted with a suitable solution to generate a single dose solution for injection. The therapeutics for the rotational combinatorial therapy described herein can be stored in lyophilized form or in solution; they can be frozen or refrigerated. In some embodiments, the lyophilized powder can contain the combination of therapeutics, such as a combination of therapeutic peptides, and additional components, such as salts, such that reconstitution with sterile distilled water results in a combination of therapeutics in a buffered or saline solution.

Unit dose forms can be administered in fractions or multiples thereof. A multiple dose form is a plurality of identical unit dosage forms packaged in a single container to be administered in segregated unit dose form. Examples of multiple dose forms include vials and syringes. Hence, multiple dose form is a multiple of unit doses that are not segregated in packaging.

The therapeutics for the rotational combinatorial therapy described herein can be provided at a concentration in the composition that is approved by regulatory agencies. For example, for FDA approved pharmaceuticals, the pharmaceuticals are formulated and provided at a concentration for which the pharmaceuticals are approved (i.e., as monotherapy or dual therapy). In some examples, the pharmaceutical composition contains multiple therapeutics in a combination and multiple different therapeutics for the rotational combinatorial therapy described herein. In some examples, the combination therapy described herein includes one or more additional agents, such as treatment of adverse side effects, or other therapeutic, for combination therapy.

### 3. Dosage and Administration

Pharmaceutical compositions suitable for use include compositions wherein the therapeutics for the rotational combinatorial therapy described herein are contained in an amount effective to achieve their intended purpose. For example, previously FDA approved medication can be in an amount or dosage as previously approved or recommended by the FDA or any other regulatory agency. In other examples, the FDA approved medication used in a combination described herein can be provided in a lower amount or dosage than approved or recommended by the FDA or other regulatory agency. In some examples, multiple medications are provided in the combination in an amount or dosage that is approved or recommended by FDA. In other examples, multiple medications are provided in the combination in an amount or dosage that is less than the amount or dosage approved or recommended by FDA. Determination of a therapeutically effective amount is well within the capability of those skilled in the art. Therapeutically effective dosages can be determined by using in vitro and in vivo methods, and/or by a skilled person.

Dosages generally start at FDA approved or recommended dosage. The lowest effective dosage can be indicated in order to decrease side effects. Dosages can be adjusted throughout the therapeutic period in accord with the regimen herein and also based on other factors. For example, dosage can be modified based on the particular subject's response and/or progress (i.e., weight loss), tolerability, volume of distribution, subject weight and/or body size, and at the discretion of the treating physician. Metabolic parameters, cardiac markers for cardiac patients (i.e., CRP, ASR), inflammatory markers (i.e., myeloperoxidase (MPO)), renal clearance also can be assessed when determining dosage; dosages to decrease inflammation, decrease cardiac risk and that are renal protective would be preferred so long as the therapeutic efficacy is maintained.

Any of the therapeutics for the rotational combinatorial therapy or combinations described herein can be formulated for single dosage or multiple dosage administration for use in the rotational therapy provided herein; generally, the therapeutics are formulated for multiple dosage. In some examples, an approved (i.e., FDA approved) therapeutic that is approved for single dosage administration is formulated for single dosage administration in the combinatorial therapy provided herein. In other examples, an approved (i.e., FDA approved) therapeutic that is approved for multiple dosage administration is formulated for multiple dosage administration in the combinatorial therapy provided herein. For example, a therapeutic that is approved for daily administration can be administered daily in the rotational combinatorial regimen described herein. In some examples of multiple dosage administration, a therapeutic can be included in a first combination and rotated into the regimen as part of a later combination (i.e., fourth or fifth combination). In other examples, a therapeutic is provided in a combination and is not rotated into the regimen again; such as a therapeutic that is provided in a first combination only, and is not included in a later combination in the regimen.

The range of doses of any of the therapeutics in the provided combinations can be formulated per kg of body weight of the subject or patient. The dose can be administered a single time during each rotation, or can be administered a multiple times. Appropriate dose amount can be determined by one of skill in the art, based on the regimen of administration. Total dose over a specific period of time can also be selected by one of skill in the art. In a rotational combinatorial therapy herein the dose can be increased over time, during the regimen. In some examples the dose can be increased or decreased during the rotation, compared to the previously administered dose.

The dose range for each of the individual therapeutics in the combinations provided herein, can be adjusted by plasma monitoring or by monitoring of symptoms or by monitoring of adverse side effects. The dose of administration can be such that the subject will maintain a plasma level to effect amelioration of symptoms of the disease, disorder, or condition for which treatment is administered. The dose of an individual therapeutic can vary depending on the other therapeutics in the combination, and can vary depending on the doses of the therapeutics in the other combinations in the rotation.

The therapeutics and combinations are included in amounts sufficient to exert a therapeutically useful effect in the absence of undesirable side effects, or with minimal or decreased side effects on the patient treated. A therapeutically effective concentration of a therapeutic(s) for treatment of any condition with a previously characterized mechanism of action or pathway can be determined empirically by testing the polypeptides in known in vitro and in vivo systems such as by using the assays provided herein or known in the art and then extrapolated therefrom for dosages for humans.

The precise amount or dose of the therapeutic agent administered depends on the particular agent, the route of administration, the other agents in the combination, the other agents previously administered in the rotational regimen, the amounts (i.e., dosages) of the other agents previously administered in the rotational regimen, and other considerations, such as the severity of the disease and the weight and general state of the subject. Local administration of the therapeutic agent will typically require a smaller dosage than any mode of systemic administration, although the local concentration of the therapeutic agent can, in some cases, be higher following local administration than can be achieved with safety upon systemic administration. If necessary, a particular dosage can be empirically determined or extrapolated. In some examples, the dosage of an approved (i.e., FDA approved) medication is the approved dosage or amount, or a standard dosage or amount, or a dosage or amount provided in applicable clinical guidelines (i.e., clinical guideline from the American Society for Radiation Oncology (ASTRO) provides guidance on the use of radiation therapy). In some examples, the dosage of a therapeutic in a combination is less than the dosage or amount approved by a regulatory agency. For example, the dosages of a therapeutic (i.e., medication) in a combination can be decreased compared to the dosage of the therapeutic when administered as a monotherapy.

The amount of any therapeutic in the combinations provided herein to be administered for the treatment of a disease or condition can be determined by standard clinical techniques. In addition, in vitro assays and animal models can be employed to help identify dosage ranges for administration. For drugs that do not have established dosages (i.e., FDA approved dosages) animal models can be used to establish an effective dosage. One of skill in the art can determine the human equivalent dose (HED), based body weight for humans. Appropriate HED can be calculated using body weight-based conversion (Reagan-Shaw et al. (2008) The FASEB Journal 22(3):659-661). The precise dosage, which can be determined empirically or based on previous use of the therapeutic (i.e., the standard dosage), can depend on the particular agent, the route of administration, the type of disease to be treated, the particular disease or condition, the particular formulation, the seriousness of the disease or condition, and other factors within the level of a skilled artisan. In some embodiments, the combinations administered can contain therapeutic peptides that mimic the peptides released or increased following bariatric weight loss surgery provided herein, such as, for example, gastrointestinal hormones, PYY and/or GLP-1. In examples, the therapeutic peptides are administered in a dosage that effects levels (i.e., circulating levels) of the peptides similar to the levels following bariatric weight loss surgery. In some examples, the dosage of an individual therapeutic in the combination is the dosage approved by a regulatory agency for administering an approved therapeutic as a monotherapy.

It is understood that the precise dosage and duration of treatment is a function of the disease being treated and the dosage and duration of treatment can follow a previously established schedule for administration of an approved therapeutic (i.e., drug). Therapeutic dosage and duration of treatment also can be determined empirically using known testing protocols or by extrapolation from in vivo or in vitro test data. One of skill in the art can, in a clinical setting, and in accord with the rotational methods and combinations described herein, determine the dosage of individual therapeutics in the combinations provided herein for administration. Dosage and duration of treatment also can be modified considering the other therapeutics in the combination, and in the rotational therapy. Concentrations and dosage values also can vary with the severity of the condition to be alleviated. The dosages can depend on the clinical response and side effect profile of an individual subject, such as a human or an animal.

For any particular subject, specific predetermined dosage regimens can be adjusted over time according to the individual need and the professional judgment of the person administering or supervising the administration of the combination therapy. The concentration ranges set forth herein are exemplary only and are not intended to limit the scope or use of combinations containing them. Generally, dosages and dosage regimens for the combination therapy are chosen to limit toxicity and/or adverse side effects. For example, the therapeutic can initially be administered at a low dosage and increased gradually according to patient's response. In another example, the therapeutic can initially be administered at the standard dosage and can be increased gradually according to the patient's response.

The combinations can be administered hourly, daily, bi-weekly, weekly, monthly, once, twice daily, three times a day, four times a day, five times a day, or more. In some examples, different therapeutics in a combination are administered with the same frequency. For example, three therapeutics in a first combination are administered once daily. In another example, four therapeutics in a first combination are administered twice daily. The frequency of administration can vary as long as all of the therapeutics are administered with the same frequency (i.e., at the same time). In other examples, therapeutics in a combination are administered on distinct schedules (i.e., with different frequency). For example, one therapeutic in a first combination is administered twice daily, and a second therapeutic in the first combination is administered once daily, and a third therapeutic in the first combination is administered bi-weekly. Generally, the therapeutics are administered in accord with their approved administration monotherapy timing. For example, therapeutics that are administered daily as a monotherapy are administered daily in the combination regimen provided herein. In other examples, therapeutics that are administered weekly as a monotherapy are administered weekly in the combination regimen provided herein. In other examples, therapeutics that are administered bi-weekly as a monotherapy are administered bi-weekly in the combination regimen provided herein.

### 4. Dosage and Administration for Treating Obesity and Overweight

In some examples of combination or rotational combinatorial therapy for treating obesity or overweight, the combinations can be formulated using previously approved (i.e., approved by the US Food and Drug Administration) dosages. For example, GLP-1 agonists can be formulated at the starting dosage in the combinations that is the starting dosage of the GLP-1 agonist administered as a monotherapy. For example, dulaglutide (Trulicity®) GLP-1 agonist can be formulated in the combination at a starting dosage of 0.75 mg administered once weekly, exenatide (sold under the trademark BYDUREON Bcise®) GLP-1 agonist can be formulated in the combination at a starting dosage of 2 mg administered once weekly, and semaglutide (soled as Ozempic®) GLP-1 agonist can be formulated in the combination at a starting dosage of 0.5 mg administered once weekly. Exemplary medications and their starting dosages are set forth in Table 11, below.

TABLE 11
Exemplary Medications, Administration and Starting Dosage for Weight Loss

| Medication | | Route | Starting Dosage | |
|---|---|---|---|---|
| i. | GLP-1 agonist | | | |
| | Trulicity ® GLP-1 agonist | SQ | 0.75 | mg per week |
| | BYDUREON Bcise ® GLP-1 agonist | SQ | 2 | mg per week |
| | Ozempic ® GLP-1 agonist | SQ | 0.5 | mg per week |
| | Victoza ® GLP-1 agonist | | | |
| ii. | Phentermine | PO | 37.5 | mg per day |
| ii. | Liothyronine | PO | 25 | mg per day |
| iv. | topiramate (Topamax ® carbonic anhydrase | PO | 25 | mg per week |
| | inhibitor) | | | |

| | | | |
|---|---|---|---|
| v. | Acarbose | PO | 25 mg-50 mg 3 times per day |
| vi. | Sitagliptin (Januvia ® dipeptidyl peptidase-4 | PO | 25 mg-100 mg per day |
| | (DPP-4) inhibitor) | | |
| vii | Canagliflozin (Invokana ® sodium-glucose | PO | 100 mg-300 mg per day |
| | co-transporter 2 (SGLT2) inhibitor) | | |
| viii. | Dapagliflozin (Farxiga ® SGLT2 inhibitor) | PO | 5 mg-10 mg per day |
| ix. | Sermorelin | SQ qhs | 20 units and 1 mg/ml |

SQ: Subcutaneous injection administration
PO: Oral administration

In some examples of combinations for rotational combinatorial therapy for treating obesity or overweight, the combinations can be formulated and administered in the therapeutic amounts or ranges set forth in Table 12, below:

TABLE 12
Exemplary Medications, Administration and Dosage Range

| Therapeutic | Range | |
|---|---|---|
| semaglutide | 3-250 | nmol/mL |
| adiponectin | 3-7 | ug/mL |
| leptin | 6-20 | ng/ml |
| oxyntomodulin | 12-30 | pmol/ |
| sermorelin | inject 200 mcg daily bid | |
| PYY | 4-370 | pg/ml |
| Amylin | 20-200 | ng/ml |
| Tesamorelin | 1 mg-2 mg/day | |
| Pancreatic polypeptide | 200-350 | pg/mL |
| Enterostatin/GIP | 250 pg/mL-370 pg/mL | |
| CCK | 6-50 | pg/mL |
| VIP | <70 | pg/mL |
| Glicentin | 8-40 | pmol/L |

Also described herein is a pharmaceutical combination for treating overweight, obesity and/or for weight loss including FDA approved medications. In some examples, one or more of the following is included the combination: 1) a GLP-1 agonist (sold under trademarks Trulicity®, B-cise®, Ozempic®, and Victoza®), 2) Phentermine, 3) Liothyronine, 4) topiramate (sold under the trademark Topamax® carbonic anhydrase inhibitor), 5) Sermorelin, and 6) tirzepatide (sold under the trademark Mounjaro™). In other examples, one or more of the following is administered: one or more GLP-1 agonists, such as, for example Trulicity®, BYDUREON Bcise®, Ozempic®, Victoza® GLP-1 agonist; Phentermine; Liothyronine; topiramate (such as Topamax® carbonic anhydrase inhibitor); Acarbose; Sitagliptin (Januvia® dipeptidyl peptidase-4 (DPP-4) inhibitor); Canagliflozin (Invokana® sodium-glucose co-transporter 2 (SGLT2) inhibitor); Dapagliflozin (Farxiga® SGLT2 inhibitor); tirzepatide; and Sermorelin. In some examples, one or more of the medications in the pharmaceutical combination is discontinued and restarted. For example, one medication (i.e., phentermine) is discontinued for a period (i.e., one month) and another medication (i.e., Liothyronine) replaces the first medication, and after a time period (i.e., 1 month) the first medication is resumed and the replacement medication is stopped. In some examples a medication or medications is started after the first medication(s) is/are administered (the regimen is started), for example 1 week, 2 weeks, 3 weeks, 4 weeks, 5 weeks, 6 weeks, 2 months, 3 months, 4 months, 5 months, 6 months or more after the regimen is started.

Accordingly, the methods and regimens provided herein include a method of treating an obese subject with a rotational combination therapy comprising administering a plurality of combinations of therapeutics to the subject. An effective amount of the therapeutics in the combinations readily is determined by one of skill in the art to effect weight loss in the subject. Doses of the therapeutics in the combinations or the timing of the treatment regimen can be varied or adjusted based on the susceptibility of the patient to the treatment, as determined by one of skill in the art, such as by using the methods provided herein.

When an individual therapeutic provided in a combination herein is co-formulated or co-administered with another therapeutic agent or agents in the combination, dosages can be provided as a ratio of the amount of one of the therapeutics in the combination to the amount of the other therapeutic agent(s) administered.

### 5. Routes of Administration of the Combinations

The therapeutics in the combination therapy and the combinations provided herein can be formulated for any route known to those of skill in the art including, but not limited to, subcutaneous, intramuscular, intravenous, intradermal, intralesional, intraperitoneal injection, epidural, vaginal, rectal, local, optic, transdermal administration, or any route of administration. For subcutaneous administration the single dosage is administered in volume that is generally 10 ml or less, such as 1-5 ml, 1-2 ml, 2-10 ml, no more than 4, 5, 6, 7, 8, or 9 ml. Formulations suited for such routes are known to one of skill in the art. Formulations of previously characterized (i.e., FDA approved) therapeutics can be administered in the combinations herein in accord with the previously characterized administration route. Therapeutic compositions also can be administered concomitantly with other therapeutics in the combination. Therapeutic compositions also can be administered with other biologically active agents including therapeutics in the combination or other biologically active agents, either sequentially, intermittently or in the same composition. The combination therapy can be administered by any suitable route, and for any use for which the combination of therapeutics are used, including treatment of obesity or overweight.

Pharmaceutical compositions can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,660; 4,769,027; 5,059,595; 5,073,543; 5,120,548; 5,354,556; 5,591,767; 5,639,476; 5,674,533 and 5,733,566). Various delivery systems are known and can be used to administer selected compositions, are contemplated for use herein, and such particles can be easily made.

The route of administration is in accord with known methods, e.g., injection or infusion by intravenous, intraperitoneal, intracerebral, intramuscular, subcutaneous, intraocular, intraarterial, intrathecal, inhalation or intralesional routes, topical, rectal, mucosal, and by sustained release systems. The combinations containing the therapeutics for the rotational combinatorial therapy described herein can be administered continuously by infusion or by bolus injection. The skilled physician can administer the combinations containing the therapeutics for the rotational combinatorial therapy described herein in a local or systemic manner.

### 6. Administration of Combinatorial Treatments for Improved Weight Loss

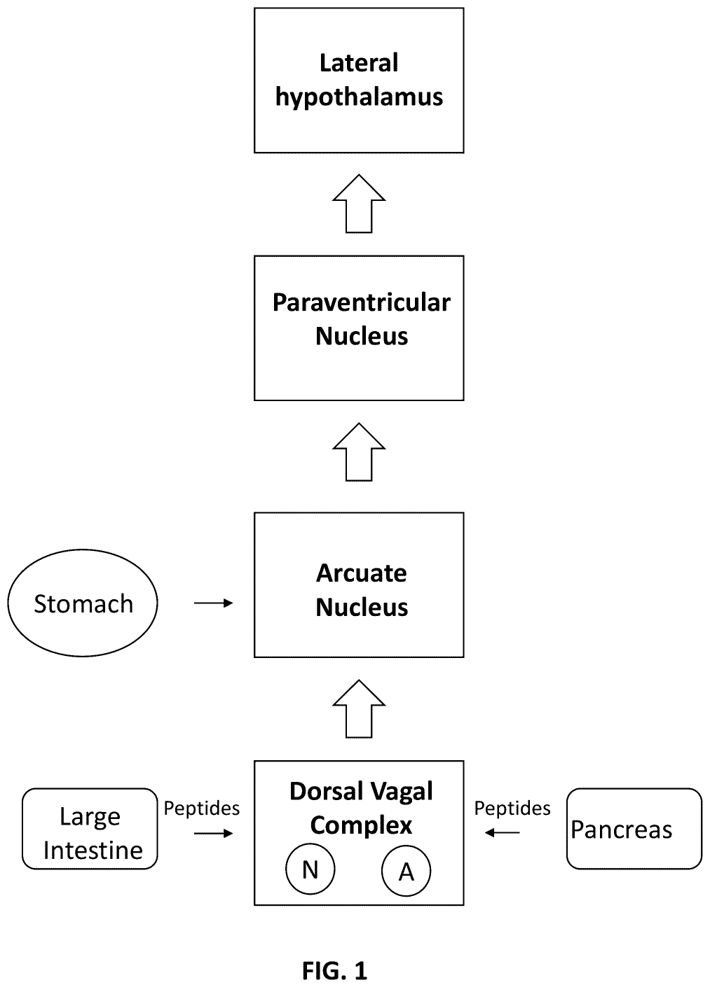

Described herein is an improved pharmacotherapeutic cocktail that mimics bariatric weight loss surgery and includes combining the selected peptides at the right physiologic time (i.e., prior to meals, or early or later in the day) and developing stable and soluble peptides that will be easily administered through subcutaneous injection, or intramuscular injection, or by oral formulation. Each of the peptides described herein form a part of the message which becomes integrated at different levels of the brain as depicted in FIG. 1 and produces an outcome of either feed or do not feed.

Table 13 below sets forth an exemplary protocol for administering a combinatorial therapy, including 6 different combinations, each comprising more than one therapeutics, where the combinations are rotated after 4 months of administration. The therapeutics in the combinations target different pathways, for different effects.

TABLE 13
Exemplary Timing for Peptide Administration

| Timing of | Peptide | |
|---|---|---|
| Administration | Combination | Function |
| Months 1-3 | | |
| AM | Peptide #1 | Appetite suppression |
| | Peptide #2 | Increase insulin sensitivity |
| | Peptide #3 | Accelerate Lipolysis and |
| | Peptide #4 | Glycogenolysis |
| PM | Peptide #5 | Lipolysis and muscle |
| | Peptide #11 | enhancement |
| Months 4-6 | | |
| AM | Peptide#6 | Appetite suppression |
| | Peptide #2 | Increase insulin sensitivity |
| | Peptide #3 | Accelerate Lipolysis and |
| | Peptide#7 | Glycogenolysis |
| PM | Peptide #8 | Lipolysis and muscle |
| | Peptide #12 | enhancement |
| Months 7-9 | | |
| AM | Peptide#9 | Appetite suppression |
| | Peptide #2 | Increase insulin sensitivity |
| | Peptide #3 | Accelerate Lipolysis and |
| | Peptide#10 | Glycogenolysis |
| PM | Peptide #5 | Lipolysis and muscle enhancement |
| | Peptide #13 | |

Peptide1 = GLP-1
Peptide 2 = Adiponectin
Peptide 3 = Leptin
Peptide 4 = Oxyntomodulin
Peptide 5 = Sermorelin
Peptide 6 = PYY
Peptide 7 = Amylin
Peptide 8 = tesamorelin
Peptide 9 = Pancreatic peptide
Peptide 10 = Enterostatin/GIP (Gastroinhibitory Polypeptide)
Peptide 11 = CCK Peptide
Peptide 12 = Vasoactive Intestinal Peptide
Peptide 13 = Glicentin CCK, Vasoactive Intestinal Peptide (VIP)

### 7. Articles of Manufacture and Kits

Pharmaceutical compositions containing the combinations for the rotational therapy described herein can be packaged as articles of manufacture containing packaging material, a pharmaceutical composition which is effective for treating a disease or condition that can be treated by rotational administration of the particular combinations, such as the diseases and conditions described herein or known in the art, and a label that indicates that the cargo, such as a drug or other therapeutic (i.e., a weight loss medication), is to be used for treating the condition, disease or disorder. The pharmaceutical compositions can be packaged in unit dosage forms containing an amount of the pharmaceutical composition for a single dose or multiple doses. The packaged compositions can contain a lyophilized powder of the pharmaceutical compositions containing the combinations for the rotational therapy which can be reconstituted (e.g., with water or saline) prior to administration in accord with the rotational regimen.

The articles of manufacture provided herein contain packaging materials. Packaging materials for use in packaging pharmaceutical products are well-known to those of skill in the art (see, e.g., U.S. Pat. Nos. 5,323,907, 5,052,558 and 5,033,252). Examples of pharmaceutical packaging materials include, but are not limited to, blister packs, bottles, tubes, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment. For example, the combinations provided herein can be packaged in accord with the packaging of the individual medications as provided individually, or can be packaged in a different packaging that contains all or some of the components of the combination

The combinations for rotational therapy can be packaged as kits. Kits optionally can include one or more components such as instructions for use, devices, and additional reagents (e.g., sterilized water or saline solutions for dilution of the compositions and/or reconstitution of lyophilized protein), and components, such as tubes, containers and syringes for practice of the methods. Exemplary kits can include the therapeutic(s) in the combinations for rotational therapy provided herein, and can optionally include instructions for use, a device, such as a syringe or other injector, for administering the combinations for rotational therapy to a subject, and a device for administering an additional therapeutic(s) agent to a subject.

The kit can, optionally, include instructions. Instructions typically include a tangible expression describing the combinations for rotational therapy, and, optionally, other components included in the kit, and methods for administration, including methods for determining the proper state of the subject, the proper dosage amount, dosing regimens, and the proper administration method for administering the combinations for rotational therapy. Instructions also can include guidance for monitoring the subject over the duration of the treatment time.

Kits also can include a pharmaceutical composition described herein and an item for diagnosis. For example, such kits can include an item for measuring the concentration, amount, or activity of therapeutic(s) in the combination for rotational therapy, in a subject.

Kits provided herein also can include a device for administering the combinations to a subject. Any of a variety of devices known in the art for administering medications to a subject can be included in the kits provided herein. Exemplary devices include, but are not limited to, a hypodermic needle, an intravenous needle, a catheter, a nebulizer, and an inhaler. Typically, the device for administering the compositions is compatible with the desired method of

### H. METHODS OF ASSESSING ACTIVITY, BIOAVAILABILITY AND PHARMACOKINETICS

### Pharmacokinetics and Tolerability

Pharmacokinetic and tolerability studies can be performed using animal models or can be performed during clinical studies with patients to assess the effect of the combinations provided herein. Animal models include, but are not limited to, mice, rats, rabbits, dogs, guinea pigs and non-human primate models, such as cynomolgus monkeys or rhesus macaques. In some instances, pharmacokinetic and tolerability studies are performed using healthy animals. In other examples, the studies are performed using animal models of a disease for which therapy with a combination herein is considered, such as animal models of obesity, or animals with metabolic disorders (i.e., transgenic animals that model or show characteristics of metabolic disorders). In other examples, animal models of diabetes (e.g., diabetes mellitus type 2), cardiovascular disease (i.e., cardiovascular disease leading to heart attack or stroke), high blood pressure, high blood cholesterol, high triglyceride levels, persisting neurodegenerative disorders, such as Parkinson's disease, metabolic syndrome, obstructive sleep apnea, cancer, osteoarthritis, depression, and/or non-alcoholic fatty liver disease can be used to assess pharmacokinetics of the combinations herein.

The pharmacokinetic properties and activities of therapeutics in the rotational combinatorial therapy herein can be assessed by direct and indirect methods. For example, the concentration of the therapeutics (i.e., drug), such as, for example, in the blood or serum or other body fluid, can be assessed after therapeutic administration or at a time point following administration, such as, for example, at or about 1 hour, 2 hours, 3 hours, 6 hours, 12 hours, 24 hours, 2 days, 3 days, 4 days, 5 days, 6 days, 7 days, 2 weeks, 3 weeks, 4 weeks or more after administration of the rotational combinatorial therapy. For example, the concentration of the active drug ingredient in the blood can be assessed. In other examples, the equation of bioavailability can be used for assessing the therapeutic effect. In some examples the following formula is used for evaluation: [AUC]ₜₑₛₜ D_{std} τₜₑₛₜ/[AUC]_{std} Dₜₑₛₜ τ_{std}, where D is the dose administered, test and std are the test and standard doses of the same drug to determine the relative availability and AUC=area under the curve.

Indirect methods also can be used to assess the pharmacokinetic properties and activities of therapeutics, and the therapeutic effect(s). For example, the rate of urinary excretion, such as cumulative urinary excretion, can be measured or by the pharmacological effects can be assessed. For example, the drug pharmacokinetics can be assessed by evaluating drug absorption, distribution, bioavailability, metabolism and elimination.

A range of doses and different dosing frequency of dosing can be administered in the pharmacokinetic studies to assess the effect of increasing or decreasing concentrations of the therapeutics in the combinations. Pharmacokinetic properties, such as bioavailability, of individual drugs in the combination also can be assessed with co-administration of other therapeutic(s) in the combination. For example, animal models can be administered the therapeutics in a first combination, using one or more routes of administration. Such studies can be performed to assess the effect of co-administration of the plurality of therapeutics (i.e., drugs) in the combination.

Studies to assess safety and tolerability also are known in the art and can be used herein. Following administration of a the combination and compositions herein, the development of any adverse reactions can be monitored. Adverse reactions can include, but are not limited to, injection site reactions, such as edema or swelling, headache, fever, fatigue, chills, flushing, dizziness, urticaria, wheezing or chest tightness, nausea, vomiting, rigors, back pain, chest pain, muscle cramps, seizures or convulsions, changes in blood pressure and anaphylactic or severe hypersensitivity responses. Typically, a range of doses and different dosing frequencies are be administered in the safety and tolerability studies to assess the effect of increasing or decreasing concentrations of chemotherapeutic agent and/or anti-hyaluronan agent, such as a polymer-conjugated hyaluronan-degrading enzyme, and/or tumor-targeted taxane in the dose.

### I. EXAMPLES

The following examples are included for illustrative purposes only and are not intended to limit the scope of the invention.

### Example 1

### Evaluation of FDA Approved Weight Loss Medications

### Comparative Example

This Example provides weight loss and other data achieved with existing weight loss drugs/medications and protocols for comparison with the results achieved using the combinatorial method provided herein in which combinations of drugs are administered, and in which the combinations can be rotated.

For comparison with results set forth in Example 2, and described throughout, weight loss in subjects treated with a variety of FDA approved weight loss medications and off label use of other medications that result in weight loss, and bariatric weight loss surgery (i.e., gastric bypass surgery) are described. Results are shown in Table 14, which sets forth, the length of the protocol, the average weight loss in percent of total weight, and the percentage of participants who achieved either greater than 5% and 10% of total weight loss at the end of the study. The results for each medication and gastric bypass surgery are shown. The highest weight loss percentage was achieved after gastric bypass surgery. Treatment with the various FDA approved weight loss medications and off label use of other medications resulted in weight loss, ranging from approximately 6% to 11% loss.

TABLE 14
Weight reduction comparison among FDA
approved weight loss medications

| | | Weight | 5% and | 10% and |
|---|---|---|---|---|
| Medication | Duration | loss | more | more |
| Phentermine | 12 weeks | 7% | — | |
| | 20 weeks | 9% | — | |
| | 28 weeks | 6% | — | |
| | 12 weeks | 8% | 95% | 62.5% |
| Orlistat | 56 weeks | 6.2% | 48% | 17.9% |
| | 56 weeks | 4% | — | |
| | 56 weeks | 8.5% | — | |
| Qysimia | 56 weeks | 10.2% | 70% | 48% |
| | 56 weeks | 10.9% | 66% | 47% |
| Locasarin | 56 weeks | 5.8% | | |
| | 56 weeks | 5.8% | | |
| Naltrexone- | 56 weeks | 6.1% | 48% | — |
| Bupropion | 56 weeks | 9.3% | 66% | 41.5% |
| Liraglutide | 56 weeks | 5.8% | 50.5% | |
| | 56 weeks | 8.4% | 54.3% | 25.2% |
| Gastric-bypass | 12 weeks | 13.7%14.5% | 100% | 90% |

Of these treatments, gastric bypass is the most effective weight loss protocol. It, however, is fraught with adverse side-effects, including, for example, chronic malabsorption, risk of internal hernias, dumping syndrome, and weight rebound. The combinatorial methods provided herein as exemplified in the Examples, provide results comparable to gastric bypass surgery, but without the need for invasive surgery.

### Example 2

### Weight loss and Safety Regimen using a Combination Pharmacological Treatment

This example describes and details exemplary combinatorial weight loss protocols and the results achieved. In general, the protocols are designed mimic the metabolic and physiological effects of bariatric weight loss surgery (i.e., gastric bypass surgery) by selecting combinations medications and treatments that mimic the metabolic and physiological effects of bariatric weight loss surgery (i.e., gastric bypass surgery).

To achieve this a set of weight loss drugs are selected and combinations of the drugs are administered. The combinations of drugs are a subset of the selected drugs. Different sets of combinations of the drugs can be rotated. By virtue of the combinatorial methods herein development of tolerance or desensitization to the medications/drugs is avoided. As a result, the ceiling of about a 5% to 10% weight loss that occurs with monotherapy protocols, is avoided. Weight loss comparable to or more than weight loss surgery, such as gastric bypass, is achieved.

The combinatorial protocol exemplified below and described herein employs combinations of drugs include that peptides that are involved in regulation of the gut-brain axis. As shown herein, these protocols are more effective than the monotherapies.

A weight loss regimen, described below, in which combinations of medications described below, were administered to human subjects. The protocol was conducted in Punta Gorda, Florida. 96 subjects signed informed consent documents and were enrolled in the weight loss regimen.

a. Subjects

The protocol was performed with the consent of each participant. Each participant understood that medications provided were being used “off label” and that they could opt out for standard of care, including monotherapy or counseling on diet and exercise. Benefits of the protocol were explained to the participants prior to initiation of the protocol, participants were counseled on general exercise, nutrition, and portion control, and medications were adjusted as needed. All subjects had failed standard of care protocols for weight management, which included intervention (i.e., surgical intervention) in some subjects.

67 female participants ages ranging from 35-78 years of age and 29 male participants ranging from 46-75 years of age finished the initial 3-month period (Table 15). Thus, of the 96 total participants, about 70% were female and 30% were male, and the average age was 59 years old. For comparison, the average age of a bariatric subject is 42 years of age and 83% of subjects are female (see, American Journal of Nursing (2012) 112(9): 26-36; Turchiello da Silva et al., Arq Bras Cir Dig. 28(4): 270-273 (2015)).

Subjects were seen once weekly for the first 4 weeks, and then once every two weeks, for six visits, for a total of 10 visits over 16 weeks. Vital signs and side effects were assessed at each visit. No participants stopped the regimen during the initial 3-month period due to side effects. The most reported side effects include constipation (40% of participants), dry mouth (35% of participants), tremulousness (10%) and insomnia (5%). One male and one female were discontinued prior to completion of the first month due to social circumstances that prevented timely follow-ups. These subjects were excluded from the demographic information, set forth in Table 15, below.

TABLE 15
Subjects' Demographics for first 12 weeks of the regimen

| | | | | | Weight | Avg. |
|---|---|---|---|---|---|---|
| | Total | | Age | Avg. | Range | Weight |
| | participants | Withdrew | range | Age | (kg) | (kg) |
| female | 67 | 1 | 35-78 | 61 | 59.2-154 | 120 |
| Male | 29 | 1 | 46-75 | 58 | 82-164 | 131 |

38% of subjects who completed the initial weeks of the regimen withdrew after the initial 12 weeks. Most participants who decided not to progress past the initial 3-month period did so because of limited finances. For example, one couple did very well in the initial 12-week period and decided they no longer needed any further follow ups as they had acquired all the information they needed to maintain their weight loss. The number of subjects who completed the nine-month (36 week) period is set forth in Table 16, below:

TABLE 16
Percent of Subjects Continuing to the
End of the Protocol (at 36 weeks)

| | % of subjects continuing from 12 weeks to 36 weeks |
|---|---|
| Female | 63% (42/67) |
| Male | 79% (23/29) |

Because the participants were approximately 20 to 25 years older than the typical surgical weight loss subject, they had additional medical issues more common to a geriatric population. These issues can hinder weight loss or make it more challenging to lose weight, and include, for example: 1) the fact that specific habits for weight gain have had longer times to set; 2) sarcopenia due to aging that significantly slows down “metabolism;” and 3) orthopedic issues, such as osteoarthritis, which can limit mobility, which is important in weight loss and weight loss maintenance.

b. Subjects' Diet and Exercise

Subjects received exercise and nutrition counseling, adjustment of medications and assessment of vital signs at each visit for the first three months (12 weeks) of the protocol.

### 1) Diet

Subjects were instructed to adhere to a liberal diet that included low carbohydrate and high protein in the ratio of 20% carbohydrates, 50% protein, and 30% fats, when possible. After 12 weeks, subjects were instructed to increase the proportion of carbohydrates. Subjects were not instructed to count calories; subjects were instructed to be cognizant of portion size/control. Estimated calorie count for the average subject ranged from approximately 1500-2000 kcal/day.

### 2) Exercise

About 70% of female and 75% of male subjects had some advanced orthopedic limitation that reduced the time and intensity of exercise. About 50% of subjects, due to advance age and musculoskeletal issues, had to find exercise alternatives and may have reduced exercise times. Subjects were instructed to start slowly and minimally complete at least 20 minutes of exercise, three times per week. About 50% of male subjects were supplemented with testosterone. All of the subjects were administered sermorelin to increase release of growth hormone, including in the combinatorial therapy as a muscle sparing/enhancing peptide.

c. Assessment of Weight Reduction and Body Changes

Weight and BMI are poor discriminators of health. Body weight, and, thus, weight loss can be composed of water, fat and muscle and at different ratios. The higher proportion of fat relative to muscle indicates an unhealthy state that can promote inflammation and insulin resistance. Conversely, having a higher ratio of muscle mass is associated with lower insulin resistance and improved insulin sensitivity (Srikanthan et al., J Clin Endocrinol Metab. (2011) 96(9):2898-903).

Unlike previous studies and programs, which follow weight loss (i.e., total pounds lost) and body mass index (BMI: kg/m2), which are clinical measurements used to determine if subjects are overweight or obese, the combination therapy protocol in the Example assessed total weight loss, fat loss, and muscle loss/gain, which, in combination, is a more accurate predictor of health status (Gömez-Ambrosi, et al., Obesity 19, 1439-1444 (2011); G6mez-Ambrosi et al., Obesity 19, 1439-1444 (2011); Kim et al., Diabetes Care 34:504-506 (2011)).

Weight, fat and muscle mass were assessed using the InBody 520™ body composition analyzer (research grade). The data presented below are based on analyses conducted with data from the InBody 520™ body composition analyzer (Biospace, Inc.; Los Angeles, CA USA), which assesses shifts of muscle to fat mass and shifts in water weight. The InBody 520™ analyzer assesses weight, lean body mass, body fat mass, body water balance, body mass index (BMI), percentage of body fat, and muscle mass, using bioelectrical impedance. These parameters were assessed during the 36 weeks of the protocol. Subjects were weighed and assessed using InBody 520™ body composition analyzer at weekly intervals for the first four weeks, then once every two weeks for the completion of the initial 3-month period.

d. Combinatorial Pharmacological Regimen

Subjects were administered or self-administered the medications in the protocol by the appropriate route (i.e., subcutaneous injection or oral tablet/capsule) for 36 weeks. Subjects initially were administered a combination of: 1) a GLP-1 agonist (sold under trademarks Trulicity®, B-cise®, Ozempic®, and Victoza®), 2) Phentermine, 3) Liothyronine, 4) topiramate (sold under the trademark Topamax® carbonic anhydrase inhibitor), and 5) Sermorelin. The starting dosages are listed below in Table 17. The dosages were adjusted throughout the treatment period based on the subject response (i.e., weight loss), tolerability, volume of distribution, subject weight, and at the discretion of the treating physician. For example, the dosage of the GLP-1 agonists Trulicity® and Ozempic® was adjusted depending on the response to the medications. The dosage of the Bydureon BCcise® GLP-1 agonist was not adjusted since it is only produced in a 2 mg injectable dose. Phentermine was increased depending on subject response, to, for example, one 37.5 mg tablet in the morning and one-half tablet (18.75 mg total) at 2 p.m. Liothyronine, which had a starting dosage of 25 mcg, was increased to up to 100 mcg. Topamax® carbonic anhydrase inhibitor, which had a starting dosage of 25 mg, was increased to 100 mg as deemed appropriate. If no progression was noted on the initial medications listed above, then the following medications were added to the protocol: Acarbose, Januvia® dipeptidyl peptidase-4 (DPP-4) inhibitor, Invokana® sodium-glucose co-transporter 2 (SGLT2) inhibitor, and Farxiga® (dapagliflozin) in the dosages listed in Table 17, below. For example, if weight loss progress was slow and a subject was consuming large amounts of carbohydrate-rich foods, the alpha-glucosidase inhibitor, Acarbose, was added to the regimen. In another example, if the subject had normal renal function, then a SGLT2 agonist (such as a SGLT2 agonist sold under the trademark Invokana® or Farxiga® or Januvia®) was administered (SGLT2 is a low affinity sodium/glucose transporter). This was added in addition to medications i-iv and ix, below. The regimen provided a minimum of four medications, and more medications were added and/or the dosages were modified as required in subsequent visits.

TABLE 17
Exemplary medications for regimens

| Medication | | Route | Starting Dosage | |
|---|---|---|---|---|
| i. | GLP-1 agonist | | | |
| | Trulicity ® GLP-1 agonist | SQ | 0.75 | mg per week |
| | BYDUREON Bcise ® GLP-1 agonist | SQ | 2 | mg per week |
| | Ozempic ® GLP-1 agonist | SQ | 0.5 | mg per week |
| | Victoza ® GLP-1 agonist | SQ | 0.6 | mg daily |
| ii. | Phentermine | PO | 37.5 | mg per day |
| iii. | Liothyronine | PO | 25 | mcg per day |
| iv. | topiramate (Topamax ® carbonic anhydrase | PO | 25 | mg per day |

| | | | |
|---|---|---|---|
| | inhibitor) | | |
| v. | Acarbose | PO | 25 mg-50 mg 3 times per day |
| vi. | Sitagliptin (Januvia ® dipeptidyl peptidase-4 | PO | 25 mg-100 mg per day |
| | (DPP-4) inhibitor) | | |
| vii. | Canagliflozin (Invokana ® sodium-glucose co- | PO | 100 mg-300 mg per day |
| | transporter 2 (SGLT2) inhibitor) | | |
| viii. | Dapagliflozin (Farxiga ® SGLT2 inhibitor) | PO | 5 mg-10 mg per day |
| ix. | Sermorelin | SQ qhs | 20 units (1 mg/ml) |

SQ: Subcutaneous injection administration
PO: Oral administration

i. Exemplary Regimens

In some examples, medications i-iv and ix, listed above, were given initially and one or more of the initial medications was discontinued; Topamax® carbonic anhydrase inhibitor was the least tolerated, and was discontinued in some cases. In other examples, the regimen began with administration of medications i-iv and ix and other medications were added; additional medications were administered at week 2 or week 7, for example, if the subject was not responding and needed an extra pharmacological ‘push.’

As noted above, the medication dosages were adjusted throughout the treatment period as needed, and as agreed to between the treating physician and the subject. For example, if a subject was losing at least four pounds per week, typically, no dosage change was made and no new medications were added. For larger subjects (i.e., starting weight over 300 pounds) who were losing less than 6 pounds per week, medication dosages were increased or additional medications were added to the regimen. The hierarchy of decision making included 1) increase the dosage of the medications to the maximum allowable dosage or to the level of tolerability of the medication; and then 2) add a fifth or sixth medication, as needed to increase weight loss.

ii. Representative Subjects

The regimens of a representative subset of subjects are set forth below. The exemplary regimens also include examples of modifications made to the combination therapy, including the modifications described above.

For Example, subject 1, a male, received the medications set forth in Table 18, below (dosages are per day):

TABLE 18

| | | | Time on | | | Time on | | Time on | |
|---|---|---|---|---|---|---|---|---|---|
| | Starting | | starting | Modified | | modified | Modified | modified | Side |
| Medication | dosage | | dosage | dosage/day | | dosage | dosage | dosage | effect(s) |
| Phentermine | ½ tablet | | 2 weeks | 1 tablet | | 2 weeks | 1.5 tablets | Rest of | Dry mouth |
| | (18.75 mg) | | | (37.5 mg) | | | (56.25 mg) | protocol | |
| Liothyronine | 25 | mcg | 4 weeks | 50 | mcg | 2 weeks | 50 mcg | Rest of | Mild |
| | | | | | | | twice a | protocol | jitteriness |
| | | | | | | | day | | |
| Topiramate | 25 | mg | 4 weeks | 50 | mg | Rest of | — | — | None |
| | | | | | | protocol | | | |

| Sermorelin | 20 units | 36 weeks | — | | — | — | — | None |
|---|---|---|---|---|---|---|---|---|
| | (bedtime) | | | | | | | |
| GLP-1 agonist | 0.75 mg | 4 weeks | 1.5 | mg | Rest of | | | constipation |
| (sold as | per week | | | | protocol | | | |
| Trulicity ®) | | | | | | | | |

Weight loss medications, phentermine, liothyronine, and a GLP-1 agonist (sold under the trademark Trulicity®), were increased based on slower initial weight loss than desired. For example, phentermine was increased from ½ tablet per day to 1 tablet per day after 2 weeks due to less than desired weight loss. Phentermine dosage was further increased to 1½ tablets per day

Subject 1 had a starting weight of 385.6 pounds and weighed 241.5 pounds at the end of the 36-week weight loss protocol. Weight (pounds), fat and muscle loss of subject 1 after 12 weeks and 36 weeks on the weight loss protocol are set forth in Table 19 below:

TABLE 19

| | | | |
|---|---|---|---|
| 12 weeks | Weight | −61.3 | −15.90% |
| | Fat | −51.6 | −29.55% |
| | Muscle | −2 | −3.64% |
| 36 weeks | Weight | −144.1 | −37.37% |
| | Fat | −133.1 | −76.23% |
| | Muscle | −2 | −3.64% |

In another example, subject 2, a female, received the medications set forth in Table 20, below:

TABLE 20

| | | Time on | | | | Time on | | Time on | |
|---|---|---|---|---|---|---|---|---|---|
| | Starting | starting | | Modified | | modified | Modified | modified | Side |
| Medication | dosage | dosage | | dosage | | dosage | dosage | dosage | effect(s) |
| Phentermine | ½ tablet | 2 | weeks | 1 | tablet | 2 weeks | 1.5 tablets | 8 weeks | Dry mouth |
| Diethylproprion | 25 mg; | 4 | weeks | | | | | | None |
| | started at | | | | | | | | |
| | 12 weeks | | | | | | | | |
| Phentermine | | | | | | | 1.5 tablets | Rest of | Dry mouth |
| | | | | | | | Start at 17 | protocol | |
| | | | | | | | weeks | | |
| Liothyronine | 25 mcg | 4 | weeks | 50 | mcg | 2 weeks | 50 mcg | Rest of | Dizziness |
| | | | | | | | twice a | protocol | |
| | | | | | | | day | | |

| | | | | | | | | |
|---|---|---|---|---|---|---|---|---|
| Empagliflozin | 10 mg po | 36 | weeks | — | — | — | — | No UTI, |
| (sold under the | qod | | | | | | | but at high |
| trade name | | | | | | | | risk for one |
| Jardiance ®) | | | | | | | | |
| Sermorelin | 20 units at | 36 | weeks | — | — | — | — | None |
| | bedtime | | | | | | | |
| Semaglutide | 1 mg sq | 36 | weeks | — | — | — | — | constipation |
| (sold under the | per week | | | | | | | |
| trade name | | | | | | | | |
| Ozempic ®) | | | | | | | | |

po = taken orally
qod = taken every other day
sq = administered by subcutaneous injection

Medications, such as phentermine, and liothyronine were increased based on slower than desired initial weight loss. After 8 weeks on phentermine, phentermine was stopped and diethylpropion was rotated in, and subject took 25 mg diethylpropion per day for 4 weeks (weeks 13-16). At week 17, subject ceased taking Diethylpropion and resumed Phentermine treatment at 1.5 tablets per day, for the rest of the protocol.

Subject 2 previously had received a sleeve gastrectomy procedure for treatment of obesity. Following the procedure, Subject 2 regained weight and participated in the instant protocol to lose additional weight. Subject 2 had a starting weight of 168 pounds and weighed 142.6 pounds at the end of the 36-week weight loss protocol. Weight (pounds), fat and muscle loss of subject 2 after 12 weeks and 36 weeks on the weight loss protocol are set forth in Table 21 below:

TABLE 21

| | | | |
|---|---|---|---|
| 12 weeks | Weight | −13.5 | −8.04% |
| | Fat | −8.2 | −11.85% |
| | Muscle | −1.5 | −5.73% |
| 36 weeks | Weight | 25.4 | 15.12% |
| | Fat | 19.4 | −28.03% |
| | Muscle | 1.5 | −5.73% |

In another example, subject 3, a male, received the medications set forth in Table 22, below:

TABLE 22

| | | Time on | | | | Time on | | Time on | |
|---|---|---|---|---|---|---|---|---|---|
| | Starting | starting | | Modified | | modified | Modified | modified | Side |
| Medication | dosage | dosage | | dosage | | dosage | dosage | dosage | effect(s) |
| Phentermine | ½ tablet | 2 | weeks | 1 | tablet | 2 weeks | 1.5 tablets | 8 weeks | Dry mouth |
| Mirabegron (a beta- | 25 mg | 4 | weeks | — | | — | — | — | — |
| 3 adrenergic | po qod; | | | | | | | | |
| agonist; sold under | started at | | | | | | | | |
| the trademark | 12 weeks | | | | | | | | |
| Myrbetriq ®) | | | | | | | | | |
| Phentermine | | | | | | | 1.5 | Rest of | None |
| | | | | | | | tablets | protocol | |
| | | | | | | | Start at | | |
| | | | | | | | 17 weeks | | |
| Liothyronine | 25 mcg | 4 | weeks | 50 | mcg | 2 weeks | 50 mcg | Rest of | None |
| | | | | | | | twice a | protocol | |
| | | | | | | | day | | |

| | | | | | | | | |
|---|---|---|---|---|---|---|---|---|
| Empagliflozin (sold | 10 mg po | 36 | weeks | — | — | — | — | None |
| under the trademark | qod | | | | | | | |
| Jardiance ®; SGLT2 | | | | | | | | |
| inhibitor) | | | | | | | | |
| Sermorelin | 20 units | 36 | weeks | — | — | — | — | None |
| | (bedtime) | | | | | | | |
| GLP-1 agonist (sold | 1 mg sq | 36 | weeks | — | — | — | — | constipation |
| as Trulicity ®, | per week | | | | | | | |
| Saxenda ®, or | | | | | | | | |
| Ozempic ®, | | | | | | | | |
| depending on what | | | | | | | | |
| was available) | | | | | | | | |

po = taken orally
qod = taken every other day
sq = administered by subcutaneous injection

Weight loss medications (i.e., phentermine, liothyronine) were increased based on slower than desired initial weight loss. After 8 weeks on phentermine, phentermine was stopped and Mirabegron was rotated in, and subject took 25 mg Mirabegron every other day for 4 weeks (weeks 13-16). At week 17, the subject ceased taking Mirabegron and resumed Phentermine treatment at 1.5 tablets per day, for the rest of the protocol.

Subject 3 had a starting weight of 251.5 pounds and weighed 197.3 pounds at the end of the 36-week month weight loss protocol. Weight (pounds), fat and muscle loss of subject 3 after 12 weeks and 36 weeks on the weight loss protocol are set forth in Table 23 below:

TABLE 23

| | | | |
|---|---|---|---|
| 12 weeks | Weight | −37.9 | −15.07% |
| | Fat | −32.2 | −31.48% |
| | Muscle | −1.3 | −3.37% |
| 36 weeks | Weight | −54.2 | −21.55% |
| | Fat | −52.7 | −51.52% |
| | Muscle | 0.2 | 0.52% |

In another example, subject 4, a male, received the medications set forth in Table 24, below:

TABLE 24

| | | Time on | | | | Time on | | Time on | |
|---|---|---|---|---|---|---|---|---|---|
| | Starting | starting | | Modified | | modified | Modified | modified | Side |
| Medication | dosage | dosage | | dosage | | dosage | dosage | dosage | effect(s) |
| Phentermine | ½ tablet | 2 | weeks | 1 | tablet | 2 weeks | 1.5 tablets | Rest of | None |
| | | | | | | | | protocol | |
| Bupropion (sold as | 150 mg | 4 | weeks | — | | — | — | — | None |
| Wellbutrin ®) | po xr qd | | | | | | | | |
| Liothyronine | 25 mcg | 4 | weeks | 50 | mcg | 2 weeks | 50 mcg | Rest of | None |
| | | | | | | | twice a | protocol | |
| | | | | | | | day | | |

| | | | | | | | | |
|---|---|---|---|---|---|---|---|---|
| Empagliflozin/metformin | 10/1000 | 36 | weeks | — | — | — | — | No UTI but |
| (sold as Synjardy ®) | mg po | | | | | | | high risk |
| | qod | | | | | | | for UTI |
| Sermorelin | 20 units | 36 | weeks | — | — | — | — | None |
| | (bedtime) | | | | | | | |
| Acarbose | 50 mg po | 32 | weeks | — | — | — | — | Mild |
| | TID; | | | | | | | stomach |
| | started 4 | | | | | | | bloating |
| | weeks into | | | | | | | |
| | protocol | | | | | | | |
| GLP-1 agonist (sold as | 1 mg sq | 36 | weeks | — | — | — | — | constipation |
| Trulicity ®) | per week | | | | | | | |

po = taken orally
qod = taken every other day
tid = taken 3 times per day
qd = taken each day
xr = Extended release
sq = administered by subcutaneous injection

Weight loss medications (i.e., phentermine, liothyronine) were increased based on slower than desired initial weight loss.

Subject 4 had a starting weight of 489 pounds and weighed 395 pounds at the end of the 36-week month weight loss protocol. Weight, fat, and muscle loss of subject 1 after 12 weeks and 36 weeks on the weight loss protocol are set forth in Table 25 below:

TABLE 25

| | | | |
|---|---|---|---|
| 12 weeks | Weight | −53.2 | −10.88% |
| | Fat | −30.5 | −11.62% |
| | Muscle | −5.5 | −9.45% |
| 36 weeks | Weight | −94 | −19.22% |
| | Fat | −57 | −21.72% |
| | Muscle | −8.4 | −14.43% |

In another example, subject 5, a female, received the medications set forth in Table 26, below:

TABLE 26

| | | Time on | | | | Time on | | Time on | |
|---|---|---|---|---|---|---|---|---|---|
| | Starting | starting | | Modified | | modified | Modified | modified | Side |
| Medication | dosage | dosage | | dosage | | dosage | dosage | dosage | effect(s) |
| Phentermine | ½ tablet | 2 | weeks | 1 | tablet | 2 weeks | 1.5 tablets | Rest of | None |
| | | | | | | | | protocol | |
| Phendimetrazine | 25 mg po | 4 | weeks | — | | — | — | — | None |
| | TID | | | | | | | | |
| | Started at | | | | | | | | |
| | week 12 | | | | | | | | |
| Phentermine | | | | | | | 1.5 tablets | Rest of | None |
| | | | | | | | Start at 17 | protocol | |
| | | | | | | | weeks | | |
| Topiramate | 25 mg po | 2 | weeks | | | | | | Brain fog, |
| | qd | | | | | | | | nausea |
| Liothyronine | 25 mcg | 4 | weeks | 50 | mcg | 2 weeks | 50 mcg | Rest of | None |
| | | | | | | | twice a | protocol | |
| | | | | | | | day | | |

| | | | | | | | | |
|---|---|---|---|---|---|---|---|---|
| Empagliflozin/metformin | 10/1000 | 2 | weeks | — | — | — | — | Yeast |
| (sold as Synjardy ®) | mg po | | | | | | | infection |
| | qod | | | | | | | |
| Sermorelin | 20 units | 36 | weeks | — | — | — | — | None |
| | (bedtime) | | | | | | | |
| GLP-1 agonist (sold as | 1 mg sq | 36 | weeks | — | — | — | — | constipation |
| Trulicity ®) | per week | | | | | | | |

po = taken orally
qod = taken every other day
tid = taken 3 times per day
qd = taken each day
xr = Extended release
sq = administered by subcutaneous injection

Weight loss medications (i.e., phentermine, liothyronine) were increased based on slower than desired initial weight loss. After 8 weeks on phentermine, phentermine was stopped and Phendimetrazine was rotated in, and subject took 25 mg Phendimetrazine every day for 4 weeks (weeks 13-16). At week 17, subject ceased taking Phendimetrazine and resumed Phentermine treatment at 1.5 tablets per day, for the rest of the protocol.

Subject 5 had previously received a gastric bypass procedure for treatment of obesity. Following weight loss from the procedure, Subject 5 regained weight and participated in the instant protocol to lose additional weight. Subject 5 had a starting weight of 188.5 pounds and weighed 149.3 pounds at the end of the 36-week weight loss protocol. Weight (pounds), fat and muscle loss of subject 1 after 12 weeks and 36 weeks on the weight loss protocol are set forth in Table 27 below:

TABLE 27

| | | | |
|---|---|---|---|
| 12 weeks | Weight | −16.5 | −8.75% |
| | Fat | −12.3 | −12.85% |
| | Muscle | −0.9 | −3.64% |
| 36 weeks | Weight | −39.2 | −20.80% |
| | Fat | −31.3 | −32.71% |
| | Muscle | −2 | −8.10% |

e. Study Results

Subjects administered the combinatorial therapy were assessed for total weight loss, fat loss, and muscle composition using the InBody 520™ body composition analyzer (Biospace, Inc.; Los Angeles, CA USA). The results are set forth in tables 28 and 29 and are detailed below.

The combined average body weight for males and females at the start of the protocol, before administration of the combinatorial pharmacological therapy, was 122 kg. The results show that the average subject lost 31.5% fat at 36 weeks following the start of treatment. Female subjects lost less total fat and percent fat than male subjects. 36 weeks after the start of treatment, female subjects lost an average of 12.1 kg, and 30.3% fat, and male subjects lost an average of 20.4 kg, and 31.5% fat. Weight loss progressed over time in male and female subjects; total body fat loss continued to decrease from 21.4% and 22% at 12 weeks to 30.3% and 31.5% at 36 weeks for females and males, respectively. Subjects lost approximately 10 to 15 kg in the first 12 weeks; several male subjects lost 7-8 pounds (about 3-4 kg) of body fat in one week.

The results show that subjects lost an average of 22% fat mass after 12 weeks of treatment with the pharmaceutical combination administered in accord with the protocol/regimen described herein; this increased to 31.5% fat mass lost after 36 weeks. Given the differences in the demographics (i.e., age) of a typical vertical sleeve gastrectomy subject and the subjects, the weight loss induced by the regimen described herein is similar to or greater than the 2-3 pounds (about 0.9-1.3 kg) of weight loss per week or approximately 30 to 40 pounds (about 13-23 kg) the first 12 weeks after weight loss surgery. Thus, the instant regimen shows results similar to a vertical sleeve gastrectomy (26.8 kg vs. 13-23 kg) in a population of subjects that are older and, thus, can have more challenges in losing body fat. The instant regimen shows results that are better than mono- or dual therapy for treatment of obesity (see, e.g., Examples 1 and 3).

Total body weight lost, detailed above, includes fat weight, muscle weight and water weight. The studies conducted herein also assessed the amount of fat and muscle loss, using the InBody 520™ body composition analyzer. Analysis of the bioelectrical impedance data shows that the percent body weight lost after treatment with the combinatorial therapy was not due to a concomitant loss in muscle mass. Subjects treated with the combinatorial therapy described herein lost 14% and 19% total body weight at 12 and 36 weeks, respectively. The muscle loss was only 2% and 3% at these time points, whereas the fat loss was 22% and 31.5% at these time points. These results indicate that at 12 and 36 weeks, the fat loss accounted for a greater percent of the total weight lost. Taken together, these results show that the combinatorial therapy for weight loss described herein, showed high overall weight loss and preferentially decreased fat compared to muscle.

Assessment of all of these metrics (e.g., total weight loss, fat loss, and muscle loss), more accurately reflects improvement in health achieved with this combinatorial therapy. Preserving muscle mass is important for improving health, as it aids increased insulin sensitivity and increases the overall well-being of the subject. The results show that females in the instant protocol, who, as a group, had a higher average age from those in previously assessed weight loss trials, showed improved percent muscle mass between 12 and 36 weeks; the muscle loss at 12 weeks was 6% and at 36 weeks was 5%. The results also show that males in the instant protocol, who, as a group, had a higher average age than in previously assessed weight loss trials, showed improved percent muscle mass between 12 and 36 weeks; the muscle loss at 12 weeks was 5% and at 36 weeks was 3%. Most subjects had limited resistance training education but were encouraged to do resistance training at least three times per week.

Older people experience sarcopenia, a progressive skeletal muscle disorder which accelerates muscle mass loss, more commonly due to the lack of anabolic hormones. Notwithstanding this, during this catabolic process, an improvement of 2% (males) and 1% (females) in muscle mass was achieved; no muscle mass was lost between 12 weeks and 36 weeks on the instant protocol despite a high loss of weight overall. Comparable data for muscle mass loss in weight loss surgery subjects is not readily available; most weight loss studies assess only excess weight loss; muscle mass loss or gain after Roux-en-Y or vertical sleeve gastrectomy generally is not reported.

TABLE 28
Fat loss after 36 weeks in subjects who completed the regimen

| | Avg fat loss (kg) | % Avg. fat loss |
|---|---|---|
| | (36 weeks) | (36 weeks) |
| Female | −12.1 | −30.3 |
| Male | −20.4 | −36 |

TABLE 29
Results after Treatment with Combinatorial
Therapy compared to baseline

| | 12 weeks | 36 weeks |
|---|---|---|
| % weight change | −14 | −19 |
| % fat change | −22 | −31.5 |
| % muscle change | −3 | −2 |

The results show that treatment with a combinatorial therapy protocol, in which combinations of medications that target different pathways are administered, and also one in which the combinations are periodically rotated, such as every two to 4 weeks, results in total weight loss and fat loss that is not achieved with previous pharmaceutical weight loss treatments; the results are comparable to those achieved by a vertical sleeve gastrectomy. The protocol also included assessing fat mass loss/gain and muscle mass loss/gain. The results of 22% fat mass loss in 12 weeks is a substantial amount of weight loss in a geriatric cohort of participants who generally have hormonal and physical barriers to achieving weight loss.

### Example 3

### Comparative Example

This example compares the weight loss in subjects in the weight loss regimen described in the Example 2 using combinatorial pharmacological treatment described herein to other available pharmacological monotherapy treatments

### A. Combinatorial Pharmacological Treatment Compared to Semaglutide (Sold Under the Trademark Wegovy®) Weight Loss Medication

Wegovy® semaglutide weight loss medication for subcutaneous injection was approved by the U.S. Food and Drug Administration in June 2021, for chronic weight management in overweight and obese adults who also have at least one weight-related comorbidity (e.g., high blood pressure, type 2 diabetes, or high cholesterol). Semaglutide is a GLP-1 incretin hormone that plays a role in appetite and digestion. Weight loss in subjects administered Wegovy® semaglutide weight loss medication was assessed previously in at least four studies (results detailed below in Table 30) studies 1, 3 and 4 were performed in non-diabetic subjects, and study 2 was performed in diabetic subjects. Study 1 was performed in subjects with obesity or overweight with a comorbidity. Study 2 was performed in subjects with obesity or overweight with type 2 diabetes. Study 3 was performed in subjects with obesity or overweight with a comorbidity who were undergoing intensive lifestyle therapy. Subjects treated with Wegovy® semaglutide weight loss medication for 12 weeks lost an average of 5.7% of their body weight (combined results from studies 1-4).

Table 30, below, is adapted from HIGHLIGHTS OF PRESCRIBING INFORMATION for WEGOVY (semaglutide) injection, for subcutaneous use, Initial U.S. Approval: 2017, revised 06/2021, and sets forth the Wegovy™ semaglutide weight loss medication studies 1-3:

TABLE 30

| | Study 1 | | Study 2 | | Study 3 | |
|---|---|---|---|---|---|---|
| | PLACEBO | WEGOVY | PLACEBO | WEGOVY | PLACEBO | WEGOVY |
| | N = 655 | N = 1306 | N = 403 | N = 404 | N = 204 | N = 407 |
| Baseline mean | 105.2 | 105.4 | 100.5 | 99.9 | 103.7 | 106.9 |
| Body Weight | | | | | | |
| (kg) | | | | | | |
| % change from | −2.4 | −14.9 | −3.4 | −9.6 | −5.7 | −16.0 |
| baseline | | | | | | |
| (LSMean) | | | | | | |
| % difference | | −12.4 | | −6.2 | | −10.3 |
| from placebo | | (−13.3; −11.6)* | | (−7.3; −5.2)* | | (−11.8; −8.7)* |
| (LSMean) (95% | | | | | | |
| CI) | | | | | | |
| % of Patients | 31.1 | 83.5 | 30.2 | 67.4 | 47.8 | 84.8 |
| losing ≥5% | | | | | | |
| body weight | | | | | | |
| % difference | | 52.4 | | 37.2 | | 37.0 |
| from placebo | | (48.1; 56.7)* | | (30.7; 43.8)* | | (28.9; 45.2)* |
| (LSMean) (95% | | | | | | |
| CI) | | | | | | |
| % of Patients | 12.0 | 66.1 | 10.2 | 44.5 | 27.1 | 73.0 |
| losing ≥10% | | | | | | |
| body weight | | | | | | |
| % difference | | 54.1 | | 34.3 | | 45.9 |
| from placebo | | (50.4; 57.9)* | | (28.4; 40.2)* | | (38.0; 53.7)* |
| (LSMean) (95% | | | | | | |
| CI) | | | | | | |
| % of Patients | 4.8 | 47.9 | 4.3 | 25.1 | 13.2 | 53.4 |
| losing ≥15% | | | | | | |
| body weight | | | | | | |
| % difference | | 43.1 | | 20.7 | | 40.2 |
| from placebo | | (39.8; 46.3)* | | (15.7; 25.8)* | | (33.1; 47.3)* |
| (LSMean) (95% | | | | | | |
| CI) | | | | | | |

LSMean = least squares mean;
CI = confidence interval
*p < 0.0001 (unadjusted 2-sided) for superiority.

The results from the STEP 1 through 5 trials showed that semaglutide is superior at weight reduction when compared with placebo. STEP 2 compared 2.4 mg semaglutide with 1.0 mg semaglutide and determined that 2.4 mg semaglutide cause more significant weight loss than 1.0 mg semaglutide. STEP 4 investigated the discontinuation of semaglutide treatment and found that those who were started on placebo after 20 weeks of treatment with the experimental dose of semaglutide experienced weight gain of around 6 kg. There were no data on actual fat loss or muscle mass loss or retention. These and other results from the studies are set forth in Table 31, below:

TABLE 31

| | | Estimated mean | Patients with weight loss | Body weight |
|---|---|---|---|---|
| | | weight change | of: >5%, >10%, >15%, | change from |
| Trial (n) | Doses and comparators | (%) | 20% (%) | baseline (kg) |
| STEP 1 | SC semaglutide 2.4 mg | −14.9 | 86.4, 69.1, 50.5, n/d | −15.3 |
| (n = 1961) | Placebo | −2.4 | 31.5, 12.0, 4.9, n/d | −2.6 |
| STEP 2 | SC semaglutide 2.4 mg | −9.6 | 68.8, 45.6, 25.8, n/d | −9.7 |
| (n = 1210) | SC semaglutide 1.0 mg | −7.0 | 57.1, 28.7, 13.7, n/d | −2.5 |
| | Placebo | −3.4 | 28.5, 8.2, 3.2, n/d | −1.3 |
| STEP 3 | SC semaglutide 2.4 mg | −16 | 86.6, 75.3, 55.8, n/d | −16.8 |
| (n = 611) | Placebo (+intensive | −5.7 | 47.6, 27, 13.2, n/d | −6.2 |
| | behavioral therapy) | | | |
| STEP 4 | SC semaglutide (68 | −7.9 | 88.7, 79.0, 63.7, 39.6 | −7.1 |
| (n = 902) | total weeks) | | | |
| | SC semaglutide | +6.9 | 47.6, 20.4, 9.2, 4.8 | +6.1 |
| | (20 weeks), then | | | |
| | placebo (48 weeks) | | | |
| STEP 5 | SC semaglutide 2.4 mg | −15.2 | 83.3, 67.4, 56.8, 39.4 | −16.11 |
| | (104 weeks) n = 132 | | | |
| | Placebo n = 111 | −2.6 | 34.9, 12.8, 0.4, 2.8 | −2.8 |

Adapted from Singh et al., J Investig Med 70: 5-13 (2022) (see also, Wilding et al., N Engl J Med 384: 989-1002 (2021); Davies et al., Lancet Diabetes Endocrinol 397: 971-84 (2021); Wadden et al., JAMA 325: 1403-13 (2021); Rubino et al., JAMA 325: 1414-25 (2021); Kushner et al., Obesity 28: 1050-61 (2020)).

In contrast, subjects treated with the combinatorial pharmacological therapy described in Example 2 lost 12% of their body weight after 12 weeks, compared to baseline weight. Thus, subjects treated with the combinatorial pharmacological therapy described herein lost more than twice the percent body weight compared to the FDA-approved semaglutide weight loss product.

The combinatorial therapy described in Example 2 demonstrates superior weight loss compared to Wegovy® semaglutide weight loss medication treatment after 36 weeks of treatment. Subjects treated with Wegovy® semaglutide weight loss medication for 36 weeks lost an average of 19% of their body weight (combined results from studies 1-4). Subjects treated with the combinatorial therapy described in Example 2 lost 31.5% of their body weight after 36 weeks, compared to baseline weight. The results show that subjects administered the combinatorial pharmacological therapy described in Example 2 exhibited superior weight loss at both time points.

Most studies on obesity, including the studies assessing Wegovy® semaglutide as a weight loss drug, assess total weight loss. Since body fat is the most metabolically harmful tissue type, the instant studies, set forth in Example 2, also assessed body fat loss and muscle mass as a measure of health change; percent fat loss is clinically more relevant to health than total weight loss. As detailed above, the combinatorial pharmacological therapy described in Example 2 was designed to create “leanness” and adjust body composition, which is demonstrated by the preferential loss of fat compared to muscle, and also improved total weight loss compared to an FDA approved weight loss medication, Wegovy® semaglutide weight loss medication.

As demonstrated in Tables 32a, 32b, 32c and 32d, below, the initial weight loss in subjects treated with the combinatorial pharmacological therapy described in Example 2 is superior to the weight loss in subjects treated with the Wegovy® semaglutide weight loss medication; subjects lost more than 7% more weight at 12 and 36 weeks compared to the average subject administered Wegovy® semaglutide weight loss medication.

TABLE 32a
Treatment with the combinatorial treatment described in
Example 2 results in greater weight loss than semaglutide

| | Novo Nordisk Wegovy ™ | | | | |
|---|---|---|---|---|---|
| | semaglutide weight | | | | |
| | loss medication | | | | Example 2 |
| | Study | Study | Study | Study | combination |
| | 1 | 2 | 3 | 4 | treatment |
| Baseline mean body weight | 105.4 | 99.9 | 106.9 | 107.2 | 122 |
| (kg) | | | | | |
| 12 weeks % weight change | −6 | −4.5 | −7 | −5.5 | −14 |
| Baseline mean body weight | 105.4 | 99.9 | 106.9 | 107.2 | 122 |
| (kg) | | | | | |
| 36 weeks % weight change | −12 | −9 | −13 | −12 | −19 |
| 12 weeks % fat change | n/d | n/d | n/d | n/d | −22 |
| 36 weeks % fat change | n/d | n/d | n/d | n/d | −31.5 |
| 12 weeks % muscle change | n/d | n/d | n/d | n/d | −3 |
| 36 weeks % muscle change | n/d | n/d | n/d | n/d | −2 |

### Differences in the Studies Assessing the Combinatorial Pharmacological Treatment Described in Example 2 and Semaglutide (Sold Under the Trademark Wegovy®) Weight Loss Medication

There were several differences between the weight loss regimen described in Example 2, and previous studies assessing the impact of semaglutide (sold under the trademark Wegovy®) medication on treatment of overweight and obesity. For example, the average age of the subjects treated with the semaglutide (sold under the trademark Wegovy®) medication was 48 years old, compared to 59 years old for subjects administered the combinatorial pharmacological therapy described in Example 2. The starting weight of the subjects administered the combinatorial pharmacological therapy described in Example 2 was higher than the weight of the subjects administered semaglutide (sold under the trademark Wegovy®) medication (122 kg compared to 104.85 kg). The weight loss regimen detailed in Example 2 was conducted in Punta Gorda, Florida, United States, which has an older population, limiting the recruitment population to mostly older subjects. The subjects following the instant protocol were older, heavier and had several exercise-limiting orthopedic conditions. Recruitment for the instant weight loss regimen included several subjects who had failed some form of prior weight loss treatment (i.e., gastric sleeve or Roux-en-Y Gastric Bypass (RYGBP)), and who demonstrated behavioral and musculoskeletal issues that caused their recidivism. Despite the higher average age, which makes it more difficult to lose weight due to decreased mobility and muscle loss; higher starting weight, which can inhibit mobility; and failure to maintain weight loss after previous surgical treatment; subjects administered the combinatorial pharmacological therapy described in Example 2 lost more weight than subjects administered the FDA approved semaglutide (sold under the trademark Wegovy®) weight loss medication.

The dietary restrictions between the Semaglutide (sold under the trademark Wegovy®) weight loss medication trials also were different from the dietary focus in the instant weight loss regimen (see Example 2). In Wegovy® weight loss medication studies 1, 2 and 4, the subjects consumed a diet with a deficit of 500 kcal/day, and subjects in study group 3 consumed 1200-1800 kcal/day followed by 60 weeks of a reduced calorie diet. In the instant weight loss regimen, subjects were not limited to a maximum number of calories, nor instructed to maintain a calorie deficit; subjects focused on portion control rather than calorie count.

The exercise instructions between the Semaglutide (sold under the trademark Wegovy®) weight loss medication trials also were different from those set forth in Example 2. In the instant weight loss regimen, few of the subjects could meet the exercise requirement of 100 to 200 minutes per week as set forth in trial 3, or the minimum of 150 minutes per week set forth in trials 1, 2, and 4 of the Semaglutide (sold under the trademark Wegovy®) weight loss medication. The subjects in the weight loss regimen detailed in Example 2, who rarely exercised or had cardiac conditions, were instructed to start on a minimal exercise regimen of 20 minutes, three times per week.

The results from the trials of the Semaglutide (sold under the trademark Wegovy®) weight loss medication show that approximately 6.8% of subjects treated with 2.4 mg semaglutide discontinued use due to adverse reactions due to the mediation. Conversely, no participants administered the combinatorial therapy described in Example 2 stopped the regimen during the initial 3-month period due to side effects.

Despite the potential limitations associated with subject selection, age bias, and a decrease in muscle promoting exercise in the regimen, weight loss after administration of the combinatorial pharmacological therapy described in Example 2 was dramatically better than the FDA approved treatment semaglutide. Additionally, muscle loss was minimal, and low compared to the total body weight loss, during a catabolic process.

B. Comparison of Other Weight Loss Drugs with the Combinations Provided Herein

The drug tirzepatide is a GLP-1/GIP dual agonist, which activates the GLP-1 receptor, as well as the GIP, which alters energy consumption. Tirzepatide is a once-weekly GIP (glucose-dependent insulinotropic polypeptide) receptor and GLP-1 (glucagon-like peptide 1) receptor agonist. GIP has been shown to decrease food intake and increase energy expenditure. This endogenous hormone known to be upregulated post Rou-en-Y gastric bypass. When combined with GLP-1 greater effectiveness on weight loss was expected.

As discussed above, semaglutide (sold under the trademark Wegovy™) is a once-weekly injection that helps with chronic weight management when used in combination with diet and exercise. The medication was studied in people who did not have type 2 diabetes, but at a higher dose. Semaglutide (under the brand name Wegovy™) is FDA-approved for weight loss in adults with a body mass index (BMI) greater than or equal to 30 mg/kg² alone or 27 mg/kg² with at least one weight-related comorbidity (e.g., high blood pressure, high cholesterol). Semaglutide has side-effects that render it difficult to tolerate the higher doses required for weight loss. Semaglutide is a GLP-1 incretin hormone that plays a role in appetite and digestion. Incretins—hormones released by your small intestine—are sent out by the body after a meal to help lower your blood sugar by triggering insulin and blocking other sources of sugar. It also slows down how quickly food leaves your stomach (called gastric emptying). These actions result in a feeling of fullness lowering your appetite leading to weight loss. Medications like GLP-1 agonists are referred to as incretin mimetics since they “mimic” these effects.

This Example compares weight loss results using the combinations herein, as described in the Examples above, with the results of a study of semaglutide and a study of tirzepatide. The efficacy of weight loss for each study and the combinations herein are compared at two different time points, 12 (3mo) weeks and 36 (9mo) weeks. The comparison shows that the instant combinations were more effective for weight loss than either of these drugs.

### 1. Semaglutide

The semaglutide study was a 68-week trial of 1,961 patients with obesity (BMI≥30 kg/m²) or with overweight (BMI 27 kg/m²-29.9 kg/m²) and at least 1 weight-related comorbid condition, such as treated or untreated dyslipidemia or hypertension; patients with type 2 diabetes mellitus were excluded. Patients were randomized in a 2:1 ratio to either the semaglutide Wegovy® or placebo, both in conjunction with a reduced-calorie diet (˜500 kcal/day deficit) and increased physical activity (recommended to a minimum of 150 min/week).

### Primary End Points:

Mean percent change in body weight from baseline to week 68′
Percentage of patients achieving ≥5% weight loss from baseline to week 68

### Secondary End Points:

Percentage of patients achieving ≥10% weight loss from baseline to week 68
Percentage of patients achieving ≥15% weight loss from baseline to week 68
Change in waist circumference from baseline to week 68
Change in SBP from baseline to week 68

TABLE 32b
DATA Summary of STEP results 11-15

| | | Estimated | | Body weight |
|---|---|---|---|---|
| | | mean weight | Patients with weight loss | change from |
| Trial (n) | Doses and comparators | change (%) | of: >5%, >10%, >15% (%) | baseline (kg) |
| STEP 1 | SC semaglutide 2.4 mg | −14.9 | 86.4, 69.1, 50.5 | −15.3 |
| (n = 1961)11 | Placebo | −2.4 | 31.5, 12.0, 4.9 | −2.6 |
| STEP 2 | SC semaglutide 2.4 mg | −9.6 | 68.8, 45.6, 25.8 | −9.7 |
| (n = 1210)12 | SC semaglutide 1.0 mg | −7.0 | 57.1, 28.7, 13.7, | −2.5 |
| | Placebo | −3.4 | 28.5, 8.2, 3.2 | −1.3 |
| STEP 3 | SC semaglutide 2.4 mg | −16 | 86.6, 75.3, 55.8 | −16.8 |
| (n = 611)13 | Placebo (+intensive | −5.7 | 47.6, 27, 13.2 | −6.2 |
| | behavioral therapy) | | | |
| STEP 4 | SC semaglutide | −7.9 | NR | −7.1 |
| (n = 902)14 | (68 total weeks) | | | |
| | SC semaglutide (20 | +6.9 | | +6.1 |
| | weeks), then placebo | | | |
| | (48 weeks) | | | |

A goal of the study was to evaluate the efficacy of semaglutide solely as a weight loss medication. Participants were recruited based on BMI, excluding those with a diagnosis of type 2 diabetes, except STEP 2, which did not exclude patients with type 2 diabetes. The experimental dose given during the trial was 2.4 mg, delivered subcutaneously once a week. The trial program was completed in March 2021 with a total of five trials; however, the results are still pending for STEP 5.

The results from STEP 1 through 4 showed that semaglutide is superior at weight reduction when compared with placebo. STEP 2 compared 2.4 mg semaglutide with 1.0 mg semaglutide and found 2.4 mg semaglutide to cause more significant weight loss than 1.0 mg semaglutide. STEP 4 investigated the discontinuation of semaglutide treatment and found that those who were started on placebo after 20 weeks of treatment with the experimental dose of semaglutide experienced weight gain of around 6 kg.

Adverse events: Approximately 6.8% of patients treated with the semaglutide at 2.4 mg dose discontinued due to adverse reaction to the medication.

Muscle and Fat composition: There were no data on actual fat loss or muscle mass loss or retention.

### B. Tirzepatide

Tirzepatide (5 mg, 10 mg, 15 mg) achieved superior weight loss compared to placebo at 72 weeks of treatment in topline results from Eli Lilly and Company's (NYSE: LLY) SURMOUNT-1 clinical trial, with participants losing up to 22.5% (52 lb. or 24 kg) of their body weight. This study enrolled 2,539 participants and was the first phase 3 global registration trial evaluating the efficacy and safety of tirzepatide in adults with obesity, or overweight with at least one comorbidity, who do not have diabetes. Tirzepatide met the co-primary endpoints of superior mean percent change in body weight from baseline and greater percentage of participants achieving body weight reductions of at least 5% compared to placebo for both. The study also achieved all key secondary endpoints at 72 weeks.

### Primary End Points:

Mean percent change in body weight from baseline to week 72
Percentage of patients achieving ≥5% weight loss from baseline to week 72

### Secondary End Points:

Percentage of patients achieving ≥10% weight loss from baseline to week 72
Percentage of patients achieving ≥20% weight loss from baseline to week 72

TABLE 32c
DATA

| | Estimated | Patients with | Body weight |
|---|---|---|---|
| | mean weight | weight loss | change from |
| Doses and comparators | change (%) | of >5%, >20% | baseline (kg) |
| SC tirzepatide 5 mg | −15% | 85%, 30% | −16 |
| SC tirzepatide 10 mg | −19.5 | 89%, 50% | −22 |
| SC tirzepatide 15 mg | −20.9 | 91%, 57% | −24 |
| Placebo | −3.1 | 31%, 3.1% | −2 |

For the efficacy estimate, participants taking tirzepatide achieved average weight reductions of 16.0% (35 lb. or 16 kg on 5 mg), 21.4% (49 lb. or 22 kg on 10 mg) and 22.5% (52 lb. or 24 kg on 15 mg), compared to placebo (2.4%, 5 lb. or 2 kg). Additionally, 89% (5 mg) and 96% (10 mg and 15 mg) of people taking tirzepatide achieved at least 5% body weight reductions compared to 28% of those taking placebo.

Adverse events: Treatment discontinuation rates due to adverse events were 4.3% (5 mg), 7.1% (10 mg), 6.2% (15 mg) and 2.6% (placebo). The overall treatment discontinuation rates were 14.3% (5 mg), 16.4% (10 mg), 15.1% (15 mg) and 26.4% (placebo).

Muscle and Fat composition: There were no data on actual fat loss or muscle mass loss or retention.

### 3. Combinatorial Treatment

Average age: participant average age was 59 years old, over 70% of females and 75% of males had some advanced orthopedic limitation which would reduce time and intensity of exercise.
Trial design is described in the Example above: As noted, all patients had failed standard of care protocols for weight management to include surgical intervention in some. Patients were instructed on a liberal diet that included low carbohydrate and high protein diet in the ratio of 20% carbs 50% protein and 30% fats when possible. After 3months they were instructed to increase the carbs. There were no counting calories only portion control. Estimated calorie count for the average patient would range from 1500-2000 kcal/day. As for exercise, over 50% of patients mentioned above due to advance age and musculoskeletal issues had to find alternatives to exercise and may have reduced exercise times. Patients at the minimum were instructed to start slow with at least 20 mins 3×s a week.

### Primary End Points:

Mean percent change in body weight from baseline to week 12,and from baseline to week 36.
Percentage of patients achieving ≥5% weight loss from baseline to week 12,and from baseline to week 36.

### Secondary End Points:

Percentage of patients achieving ≥10% weight loss from baseline to week 12,and from baseline to week 36.
Percentage of patients achieving ≥15% weight loss from baseline to week 12,and from baseline to week 36.

TABLE 32d
DATA

| | Estimated mean | Patients with weight | Body weight |
|---|---|---|---|
| Doses and | weight change (%) | loss of >5%, >10% | change from |
| comparators | 12 wks | at 12 wks | baseline (kg) |
| cocktail | −14 | 100%, 90% | −16 |
| | Estimated mean | Patients with weight | Body weight |
| Doses and | weight change (%) | loss of >5%, >10% at | change from |
| comparators | 36 wks | 12 wks | baseline (kg) |
| cocktail | −33 | 95%, 90% | −22 |

Muscle enhancement: More than 50% of male subjects, were supplemented with testosterone and all males and females had sermorelin added to their regimens as a muscle sparing/enhancing peptide.

### 4. Differences Between the Semaglutide Study the Protocol for the Combinatorial Study

a. The age between the two groups is significantly different. The group in the StarRock Cocktail protocol were on the average 10 years older, heavier and with several exercise limiting orthopedic conditions. The protocol was conducted in Punta Gorda FL, which limited the recruitment population to older patients. The recruitment also included patients who had failed some form, whether gastric sleeve or RYGBP, which posed unique issues, to include behavioral and musculoskeletal issues that caused their recidivism.

b. There were few patients that could meet the exercise requirement of 100-200 min/week in our study group. As noted above, patients who rarely exercised or had cardiac conditions were instructed to start on a minimal exercise regimen of 20 min 3 times a week.

c. The dietary restrictions are notably different as well. In study groups 1, 2, and 4 there was a deficit of 500 kcal/day and study group 3 of 1200-1800. The focus was on portion control rather than calorie count since large discrepancies in actual calorie count could cause for erroneous data interpretation.

d. A focus was on enhancing muscle development through peptide/hormone supplementation, which in effect this reduces percent weight loss as increased muscle mass contributes to total bodyweight.

For this reason, the percent fat loss, which is clinically more relevant to health, is considered the more relevant parameter.

### 5. Results

As seen in table above, the initial weight loss in comparison to semaglutide is superior in the protocol by more than 7% at 3 months. There was on average a 22% bodyfat drop from baseline with the combinatorial protocol. This became more relevant at 9 months, where even though weight loss is a catabolic process, there was not loss of muscle mass between 3 months and 9 months, and a higher portion of the percent weight loss was fat loss. The percent fat loss increased from 22% to 31.5% during this time.

Even with the limitations above with the patient selection, age bias, and a muscle promoting component, the percentage of weight loss with the combinatorial therapy provide herein was significantly more effective and resulted in minimal muscle loss during a catabolic process.

### D. The Combinatorial Pharmacological Treatment Compared to Other Previously Characterized Treatments

Subjects administered the combinatorial pharmacological therapy described in Example 2 show greater weight loss than subjects administered phentermine, orlistat, Qsymia, Locasarin, naltrexone-bupropion, or liraglutide. The results, which are set forth in Table 33 below, compare the total weight loss and percent of subjects who lost greater than 5% (>%5) or greater than 10% (>%10). The last row labeled “Combinatorial Therapy” is the weight loss in subjects administered the therapy detailed herein in Example 2 (N=96). These results show that a combination of medications has results that are comparable to a surgical gastric bypass, but are noninvasive, and that surpass the weight loss associated with monotherapy with known weight loss medications.

TABLE 33
Treatment with the combinatorial pharmacological
treatment described herein results in greater weight
loss than previously characterized treatments

| Medication | Duration | Weight loss | >%5 | >%10 |
|---|---|---|---|---|
| phentermine | 12 weeks | 7% | — | |
| | 20 weeks | 9% | — | |
| | 28 weeks | 6% | — | |
| | 12 weeks | 8% | 95% | 62.5% |
| Orlistat | 56 weeks | 6.2% | 48% | 17.9% |
| | 56 weeks | 4% | — | |
| | 56 weeks | 8.5% | — | |
| Qysimia | 56 weeks | 10.2% | 70% | 48% |
| | 56 weeks | 10.9% | 66% | 47% |
| Locasarin | 56 weeks | 5.8% | | |
| | 56 weeks | 5.8% | | |
| Naltrexone- | 56 weeks | 6.1% | 48% | — |
| bupropion | 56 weeks | 9.3% | 66% | 41.5% |
| Liraglutide | 56 weeks | 5.8% | 50.5% | |
| | 56 weeks | 8.4% | 54.3% | 25.2% |
| Combinatorial | 12 weeks | 13.7%-14.5% | 100% | 90% |
| Therapy | | (24-27% fat loss) | | |

### Example 4

### Weight Loss and Safety Demonstration Using a Rotational Combinatorial Pharmacological Treatment

The rotational combinatorial pharmacological treatment includes a combination of therapeutic and endogenous peptides administered to human subjects. As described in the detailed description, a rotational combinatorial pharmacologic protocol for obesity that mimics the plurality of effects of gastric bypass was designed. The protocol targets multiple orexigenic pathways for downregulation by administering peptide combinations.

a. Subjects

A protocol is performed with subject consent and potential benefits and adverse effects of the protocol are identified prior to initiation. Subjects include those that had failed standard of care protocols for weight management and/or that are overweight or obese.

b. Assessment of Weight Reduction and Body Changes

Weight, fat and/or muscle mass are assessed before, during and after treatment. For example, weight, fat, and muscle mass are assessed using the InBody 520™ body composition analyzer (research grade), during the treatment period at predetermined intervals, and post-treatment.

c. Combinatorial Rotational Therapy

The combinatorial rotational therapy is self-administered by subcutaneous injection or taken orally or by another appropriate route for the particular peptide. Subjects are administered GLP-1 (a 31aa biochemical variant of semaglutide), adiponectin (244aa), Leptin (167aa), Oxyntomodulin (36aa), Sermorelin (29 aa), PYY (34aa), Amylin (37aa), tesamorelin (44aa), Pancreatic peptide e (36aa), Enterostatin/GIP (Gastroinhibitory Polypeptide) (50aa), CCK Peptide (4aa), Vasoactive Intestinal Peptide (28 aa), and/or Glicentin (69aa) in various combinations at the times and intervals set forth in Table 34, below:

TABLE 34
Exemplary Regimen for Peptide Administration

| Administration | Peptide | Target Peptide | | |
|---|---|---|---|---|
| Timing | Combination | Serum Levels | | Function |
| Months 1-3 | | | | |
| AM | GLP-1 | 5-50 | pmol/L | Appetite suppression |
| | Adiponectin | 3-7 | ug/ml | Increase insulin sensitivity |
| | Leptin | 6-20 | ng/ml | Accelerate lipolysis and |
| | Oxyntomodulin | 12-30 | pmol/L | glycogenolysis |
| PM | Sermorelin | 6-50 | pg/ml | Lipolysis and muscle |
| | CCK Peptide | | | enhancement |
| Months 4-6 | | | | |
| AM | PYY | 4-370 | pg/ml | Appetite suppression |
| | Adiponectin | | | Increase insulin sensitivity |
| | Leptin | | | Accelerate lipolysis and |
| | Amylin | 20-200 | ng/ml | glycogenolysis |
| PM | Tesamorelin | | | Lipolysis and muscle |
| | Vasoactive | <70 | pg/ml | enhancement |
| | Intestinal Peptide | | | |
| | (VIP) | | | |
| Months 7-9 | | | | |
| AM | Pancreatic peptide | 200-350 | pg/mL | Appetite suppression |
| | Adiponectin | | | Increase insulin sensitivity |
| | Leptin | | | Accelerate lipolysis and |
| | Enterostatin/GIP | 250-370 | pg/mL | glycogenolysis |
| | (Gastroinhibitory | | | |
| | Polypeptide) | | | |
| PM | Sermorelin | | | Lipolysis and muscle |
| | Glicentin | 8-40 | pmol/L | enhancement |

The peptides are administered in amounts necessary to achieve the following target peptide serum levels: semaglutide 3-250nmol/ml; adiponectin 3-7ug/ml; leptin 6-20 ng/ml; oxyntomodulin 12-30 pmol; sermorelin (inject 200 mcg daily bid); PYY 4-370pg/ml; Amylin 20-200 ng/ml; tesamorelin; pancreatic polypeptide 200-350 pg/ml; enterostatin/GIP 250pg/ml-370 pg/ml; cck 6-50pg/ml; VTP <70pg/ml; and glicentin 8-40pmol/L. The half-life of the peptides varies, for example, the variant of semaglutide has a half-life of 7 days, adiponectin has a half-life of 2.5 hours, and VTP has a half-life of 2 minutes. Each of the peptides have effects for weight loss and sustaining muscle, and most have anti-glycemic effects to ameliorate symptoms of type II diabetes.

e. Results

Subjects administered the combinatorial rotational therapy in accord with the timing set forth in Table 34, above, are assessed for total weight loss, fat loss, and/or muscle composition, for example, by using the InBody 520™ body composition analyzer (Biospace, Inc.; Los Angeles, CA USA).

### Example 5

### Weight loss using a Rotational Combinatorial Pharmacological Treatment

Exemplary rotational combinatorial treatments are described above and detailed in Tables 6.1, 6.2, 6.3, and 6.4. As another example, a subject is treated with a rotational combinatorial weight loss protocol in which a combination of at least two, typically at least three, of drugs, such as those set forth in Table 34, above, are administered for a predetermined time as a first round, and then a different combination, which contains two or three drugs, which can include a drug from the first round, but is a different combination is administered for a predetermined time, followed by a third round, and so on. One or more of the rounds can include a single drug, as long as another round includes at least two, generally at least 3 drugs. The rounds can be repeated after one regimen is completed. Table 35 below provides an exemplary rotational combinatorial regimen for weight loss, and exemplary doses. It is understood that doses can be adjusted for a particular subject depending upon parameters understood by skilled practitioners, such as height, weight, age, side-effects experienced, and other parameters

TABLE 35
Exemplary subject treatment regimen

| Weeks | Medications | Dosage | |
|---|---|---|---|
| 1-4 | Phentermine | 37.5 | mg |
| | Sermorelin | 10 | u |
| | Ozempic ® semaglutide | 0.25 | mg |
| | Cytomel ® liothyronine | 25 | mcg |
| 5-8 | ECA (ephedrine, caffeine, and aspirin) | 1 | cap |
| | Sermorelin | 10 | u |
| | Trulicity ® GLP-1 agonist | 0.75 | |
| | Cytomel ® liothyronine | 25 | mcg |
| 9-12 | Phentermine | 37.5 | |
| | AOD9604 analog of hGh lipolytic domain | 10 | u |

| | |
|---|---|
| Ozempic ® semaglutide | 0.5 |
| Januvia ® dipeptidyl peptidase-4 inhibitor | 25 |

### Example 6

### Treatment of Parkinson's Disease with a Combinatorial Rotational Pharmaceutical Therapy

Another example of a disease, disorder, or condition amenable to a combinatorial treatment protocol, and/or a combinatorial rotational treatment protocol is Parkinson's disease (PD). There are multiple targets/receptors for therapeutic intervention, and a plurality of different drugs for each target. PD is the fastest growing neurological disease in the world and is characterized by a combination of motor symptoms, including rest tremor, bradykinesia, rigidity, postural instability, stooped posture, and non-motor symptoms, including psychiatric and cognitive disorders (see, e.g., Dorsey et al., J Parkinsons Dis. (2018) 8(Suppl 1): S3-S8). Parkinson's disease is a complex, multi-receptor-based disease process. In a pharmacological sense, Parkinson's disease is similar to obesity in that it involves multiple pathways and is one in which receptor downregulation is an issue. Parkinson's disease, thus, is a disease that can be treated with a rotational combinatorial pharmacological approach by administering combinations of drugs that target different pathways or receptors and rotating different combinations of drugs.

Because of the presumed dominant role of dopamine in the striatum, pharmacological treatment of Parkinson's disease has focused on restoring dopaminergic neurotransmission and improving motor symptoms. Barriers exist to effective treatment of Parkinson's disease; licensed Parkinson's disease medications have several limitations. For example, chronic administration of Levo-dopa induces downregulation of receptors in the striatum (Reches et al., Neurology (1984) 34(9):1208-12). Also, there has been less focus on the complementary systems such as monoaminergic, noradrenergic, cholinergic, and glutamatergic systems that are altered in PD and that can be involved in a variety of motor and non-motor symptoms. In another example, randomized clinical trials primarily were designed to assess the efficacy and safety of treatments for motor fluctuations and dyskinesia, which may not effectively treat the cause or other neurological symptoms of the disease.

The combinatorial and rotational therapy described herein can be used to treat PD. This strategy targets receptors, pathways, neurotransmitters, and other molecular and cellular components and pathways associated with PD. These include, for example, combinations of dopamine and/or noradrenaline transporter inhibitors, anticholinesterase inhibitors, peroxisome-proliferator-activated-receptor-agonists, and iron chelators. These combinations target a plurality of therapeutic points of intervention that can be subject to downregulation after continuous monotherapy and/or to improve effectiveness of treatment. An exemplary combinatorial rotational therapy for PD is set forth in Table 36 below.

TABLE 36
Example of rotational and combinatorial pharmacology for PD

| | Medication Combination | Function |
|---|---|---|
| Months 1-3 | Medication #1 | Levo-carbidopa |
| | Medication #2 | Antimuscarinic |
| Months 4-6 | Medication #3 | Levo-carbidopa |
| | Medication #4 | MAO-B |
| Months 7-9 | Medication #5 | COMT |
| | Medication#6 | Dopamine |
| Key - exemplary | Medication1 = Rytary | |
| drugs with the | Medication 2 = Cogentin | |
| requisite | Medication 3 = Sinemet | |
| activity | Medication 4 = Eldepryl | |
| | Medication 5 = Comtan | |
| | Medication 6 = Mirapex | |

### Example 7

### Treatment of Chronic Obstructive Pulmonary Disease (COPD) with a Combinatorial Rotational Pharmaceutical Therapy

COPD is a chronic inflammatory state due to noxious and toxic particles that trigger a cascade of molecular events where permanent lung damage occurs. The prevalence of COPD in the US adult population (≥45 years) is about 3.6% and increases greatly with age (1.9% in the age group 45-64, 4.8% in 65-74, 6.8% in 75-84 and 5.6% in ≥85 years).

Pharmacological treatment of COPD is symptomatic and generally employs bronchodilators, such as selective 02-adrenergic agonists (short- and long-acting), anticholinergics, theophylline, and leukotriene inhibitors, corticosteroids and other immunosuppressive drugs, and some combinations of these drugs. These treatments are often ineffective and do not decrease the progressive decline in lung function in COPD subjects (Montuschi, P., Int J Chron Obstruct Pulmon Dis (2006) 1(4):409-423).

Targeting β2 receptors, anticholinergic muscarinic receptors, steroid receptors, leukotriene receptors is needed for managing the chronic COPD disease state. A commercial inhaled triple combination therapy that includes long acting β2 agonists, a long acting anti-muscarinic, and an inhaled corticosteroid has been used (i.e., the product sold under the trademark Trelegy Ellipta® inhaler, which contains fluticasone furoate, umeclidinium, and vilanterol).

As with most biological systems that involve an agonist-receptor complex, over time downregulation of the receptors reduces treatment efficacy. Administration of inhaled beta-agonists results in substantial downregulation and functional desensitization of lung cell beta 2-adrenergic receptor (AR). This evidences a dynamically regulated beta 2-AR in humans, the function of which can be attenuated in relevant target tissues by administration of standard doses of beta-agonist (Turki et al., AJP Lung (1995) 269(5):L709-L714). Upregulation is another phenomenon that occurs with chronic exposure of a biological system to an antagonist. Chronic exposure of NMDA receptors to antagonist influence leads to an upregulation of the receptor as measured by radiolabeled ligand studies (Follesa et al., J Neurosci (1996) 16(7):2172-2178).

Chronic ongoing receptor agonist or antagonist stimulation leads to either tolerance or hypersensitivity to the delivered medication. By rotating medications, for example with other medications that have different K_{d} (dissociation co-efficient) due to a weaker or stronger affinity for the receptor, allows for continued efficacy of multiple medications despite continued treatment. An exemplary combinatorial rotational therapy for COPD is set forth in Table 37, below.

TABLE 37
Example of rotational and combinatorial pharmacology for COPD

| | Medication Combination | Activity |
|---|---|---|
| Months 1-3 | Medication #1 | Steroid |
| | Medication #2 | Long-acting B agonist |
| | Medication #3 | Leukotriene inhibitor |
| | Medication #4 | Anticholinergic |
| Months 4-6 | Medication #5 | Steroid |
| | Medication #6 | Long-acting B agonist |
| | Medication #7 | Leukotriene inhibitor |
| | Medication #8 | Anticholinergic |
| Key - | Medication 1 = Budesonide | |
| exemplary | Medication 2 = Fometerol | |
| drugs with | Medication 3 = Monteleukast | |
| the requisite | Medication 4 = Glycopyrrolate | |
| activity | Medication 5 = Mometasome | |
| | Medication 6 = Salmeterol | |
| | Medication 7 = Moneleukast | |
| | Medication 8 = Umeclidium | |

These combinations, and other combinations, can be cycled, so that the combination from months 1-4 can be administered in months 6-9. Other combinations of these medications can be cycled as appropriate.

### Example 8

### Treatment of Hypertension with a Combinatorial Rotational Pharmaceutical Therapy

Hypertension involves inflammation and numerous receptors. Alpha-1 and alpha-2 receptors, dopamine-1 and dopamine-2, beta-1, beta-2, and beta-3, along with nitric oxide are known receptor-mediated mechanisms that can regulate blood pressure. A combination of medications has been used clinically to control blood pressure to acceptable levels. Combination medications can counter waning efficacy of the primary agents and can activate a secondary receptor to reduce blood pressure when efficacy of the primary agent wanes.

As with Parkinson's Disease and COPD, chronic exposure of an agonist or an antagonist can up- or down-regulate a targeted receptor. Evidence indicates that a-adrenergic receptors in tissues can undergo down-regulation by agonists and up-regulation by antagonists. For example, prolonged infusion with epinephrine or norepinephrine decreases renal a1 (but not renal a2)-adrenergic receptors (Snavery et al., Endocrinology (1985) 117:2182-2189) and treatment with the a2-selective antagonist yohimbine can up-regulate renal a2-adrenergic receptors (Saiz et al., Eur J Pharmacol (1986) 121:161-166 78).

A treatment strategy to continue to regulate blood pressure effectively in subjects who fail polypharmacy for control, is to add a rotational component to the combination therapy. For example, a rotational combinatorial therapy containing a variety of alpha-2 agonist, alpha-1 antagonists, beta blockers, and drugs with specificity for differing receptor subtypes can effectively reduce and maintain blood pressure.

Previously characterized drugs that can be used for a rotational combinatorial pharmacological treatment, such as a treatment with multiple medications that are rotated to prevent down-regulation of the drug targets are provided in Table 38, below. For example, non-selective α-adrenergic receptor antagonists, selective α₁-adrenergic receptor antagonists, and selective α₂-adrenergic receptor agonists can be included in a combination treatment and rotated for the treatment of hypertension.

Non-selective α-adrenergic receptor antagonists include, for example, phenoxybenzamine, phentolamine, tolazoline, and trazodone. Selective α₁-adrenergic receptor antagonists include, for example, Alfuzosin, Doxazosin, Prazosin (inverse agonist), Tamsulosin, terazosin and Silodosin. Selective α₂-adrenergic receptor agonists include, for example, clonidine, guanfacine and alpha methyl-dopa. See Table 38.

TABLE 38
Example of rotational and combinatorial
pharmacology for treating hypertension

| | Peptide combination | Activity |
|---|---|---|
| Months 1-3 | Medication #1 | Nonselective alpha blocker |
| | Medication #2 | Alpha-1 antagonist |
| | Medication#3 | Alpha-2 agonist |
| Months 4-6 | Medication #4 | Beta-1 blocker |
| | Medication #5 | Alpha-1 antagonist |
| | Medication #6 | Mixed alpha beta antagonist |
| | Medication #7 | Nonselective alpha blocker |
| Key | Medication 1 = | |
| | Phenoxybenzamine | |
| | Medication 2 = Doxazosin | |
| | Medication 3 = Clonidine | |
| | Medication 4 = Metoprolol | |
| | Medication 5 = Terazosin | |
| | Medication 6 = Carvedilol | |
| | Medication 7 = Phentolamine | |

### Sequence Summary

The following table summarizes sequences of peptides and other polypeptides. Precursors and peptides are noted in the tables.

| | | | Signal | Mature | Precursor or |
|---|---|---|---|---|---|
| SEQ | | | Peptide | Peptide | Propeptide |
| ID No. | Peptide Name | Full Sequence Residues | Residues | Residues | Residues |
| 1 | Glucagon-like | HDEFERHAEGTFTSDVSSYLEG | n/a | 8-36 | 1-6; 37 |
| | peptide-1 (GLP-1) | QAAKEFIAWLVKGRG | | | |
| 2 | Adiponectin | MLLLGAVLLLLALPGHDQETTT | 1-14 | 15-244 | n/a |
| | | QGPGVLLPLPKGACTGWMAGI | | | |
| | | PGHPGHNGAPGRDGRDGTPG | | | |
| | | EKGEKGDPGLIGPKGDIGETGV | | | |
| | | PGAEGPRGFPGIQGRKGEPGE | | | |
| | | GAYVYRSAFSVGLETYVTIPNM | | | |
| | | PIRFTKIFYNQQNHYDGSTGKF | | | |
| | | HCNIPGLYYFAYHITVYMKDVK | | | |
| | | VSLFKKDKAMLFTYDQYQENN | | | |
| | | VDQASGSVLLHLEVGDQVWLQ | | | |
| | | VYGEGERNGLYADNDNDSTFT | | | |
| | | GFLLYHDTN | | | |
| 3 | Leptin | MHWGTLCGFLWLWPYLFYVQ | 1-21 | 22-167 | n/a |
| | | AVPIQKVQDDTKTLIKTIVTRIN | | | |
| | | DISHTQSVSSKQKVTGLDFIPGL | | | |
| | | HPILTLSKMDQTLAVYQQILTS | | | |
| | | MPSRNVIQISNDLENLRDLLHV | | | |
| | | LAFSKSCHLPWASGLETLDSLG | | | |
| | | GVLEASGYSTEVVALSRLQGSL | | | |
| | | QDMLWQLDLSPGC | | | |
| 4 | Oxyntomodulin | HSQGTFTSDYSKYLDSRRAQDF | n/a | 1-37 | n/a |
| | (OXY/OXM) | VQWLMNTKRNRNNIA | | | |
| | | (see below for modified | | | |
| | | sequence with K at residue | | | |
| | | 33) | | | |
| 4 | Oxyntomodulin | HSQGTFTSDYSKYLDSRRAQDF | n/a | 1-37 | n/a |
| | (OXY/OXM) | VQWLMNTKRNKNNIA | | | |
| | | (with K at residue 33) | | | |
| 5 | Sermorelin | YADAXFXNSYRKVLGQLSARKL | n/a | 1-29; | n/a |
| | | LQDXMSR; | | 5X is I; | |
| | | YADAIFTNSYRKVLGQLSARKLL | | 7X is T; | |
| | | QDIMSR, where residue 5 is I, | | 26X is | |
| | | 7 is T, and 26 is X | | | |
| 6 | Peptide tyrosine- | MVFVRRPWPALTTVLLALLVCL | 1-28 | 29-64 | 65-97 |
| | tyrosine (PYY) | GALVDAYPIKPEAPREDASPEEL | | | |
| | | NRYYASLRHYLNLVTRQRYGKR | | | |
| | | DGPDTLLSKTFFPDGEDRPVRS | | | |
| | | RSEGPDLW | | | |
| 7 | Amylin | KCNTATCATQRLANFLVHSSNN | n/a | 1-37 | n/a |
| | | FGAILSSTNVGSNTY | | | |
| 8 | Tesamorelin | YADAIFTNSYRKLGQLSARKLLQ | n/a | 1-43 | n/a |
| | | DIMSRQQGESNQERGARARL | | | |
| | | (see below (44 amino acids | | | |
| | | long) includes a V at residue | | | |
| | | 13) | | | |
| 8 | Tesamorelin | YADAIFTNSYRKVLGQLSARKLL | n/a | 1-44 | n/a |
| | (32-75) | QDIMSRQQGESNQERGARARL | | | |
| | | (with V at residue 13) | | | |
| 9 | Pancreatic | MAAARLCLSLLLLSTCVALLLQP | 1-29 | 30-63 | 64-105 |
| | polypeptide | LLGAQGAPLEPVYPGDNATPE | | | |
| | | QMAQYAADLRRYINMLTRPSA | | | |
| | | CPCCLFPPRYGKRHKEDTLAFSE | | | |
| | | WGSPHAAVPRELSPLDL | | | |
| 10 | Gastric inhibitory | MVATKTFALLLLSLFLAVGLGEK | 1-21 | 52-93 | 22-50; |
| | polypeptide (GIP) | KEGHFSALPSLPVGSHAKVSSP | | | 95-153 |
| | | QPRGPRYAEGTFISDYSIAMDKI | | | |
| | | HQQDFVNWLLAQKGKKNDW | | | |
| | | KHNITQREARALELASQANRKE | | | |
| | | EEAVEPQSSPAKNPSDEDLLRD | | | |
| | | LLIQELLACLLDQTNLCRLRSR | | | |
| 11 | cholecystokinin | MNSGVCLCVLMAVLAAGALT | 1-20 | 46-103 | 21-45; |
| | (CCK) | QPVPPADPAGSGLQRAEEAPR | | | 104-115 |
| | | RQLRVSQRTDGESRAHLGALLA | | | |
| | | RYIQQARKAPSGRMSIVKNLQ | | | |
| | | NLDPSHRISDRDYMGWMDFG | | | |
| | | RRSAEEYEYPS | | | |
| 12 | Vasoactive | MDTRNKAQLLVLLTLLSVLFSQ | 1-20 | 81-152 | 21-80; |
| | intestinal peptide | TSAWPLYRAPSALRLGDRIPFE | | | 109-170 |
| | | GANEPDQVSLKEDIDMLQNAL | | | |
| | | AENDTPYYDVSRNARHADGVF | | | |
| | | TSDFSKLLGQLSAKKYLESLMGK | | | |
| | | RVSSNISEDPVPVKRHSDAVFT | | | |
| | | DNYTRLRKQMAVKKYLNSILNG | | | |
| | | KRSSEGESPDFPEELEK | | | |
| 13 | Glicentin | MKSIYFVAGLFVMLVQGSWQR | 1-20 | 21-89; | 90-91; |
| | | SLQDTEEKSRSFSASQADPLSDP | | 92-128; | 129-130; |
| | | DQMNEDKRHSQGTFTSDYSKY | | 131-143; | 144-145; 179- |
| | | LDSRRAQDFVQWLMNTKRNR | | 146-178 | 180 |
| | | NNIAKRHDEFERHAEGTFTSDV | | | |
| | | SSYLEGQAAKEFIAWLVKGRGR | | | |
| | | RDFPEEVAIVEELGRRHADGSF | | | |
| | | SDEMNTILDNLAARDFINWLIQ | | | |
| | | TKITDRK | | | |
| 14 | AGRP (agouti- | MLTAAVLSCALLLALPATRGAQ | 1-20 | 83-132 | 21-82 |
| | related peptide) | MGLAPMEGIRRPDQALLPELP | | | |
| | | GLGLRAPLKKTTAEQAEEDLLQ | | | |
| | | EAQALAEVLDLQDREPRSSRRC | | | |
| | | VRLHESCLGQQVPCCDPCATCY | | | |
| | | CRFFNAFCYCRKLGTAMNPCSR | | | |
| | | T | | | |
| 15 | AZP-531 | RVQSPEHQ | n/a | 1-8 | n/a |
| 16 | CAT (cocaine and | MESSRVRLLPLLGAALLLMLPLL | 1-27 | 28-116 | n/a |
| | amphetamine- | GTRAQEDAELQPRALDIYSAVD | | | |
| | regulated | DASHEKELIEALQEVLKKLKSKR | | | |
| | transcript, | VPIYEKKYGQVPMCDAGEQCA | | | |
| | encoded by | VRKGARIGKLCDCPRGTSCNSF | | | |
| | CARTPT gene) | LLKCL | | | |
| 17 | Davalintide | KCNTATCVLGRLSQELHRLQTY | n/a | 1-32 | n/a |
| | (AC2307) | PRTNTGSNTY | | | |
| 18 | Enterostatin | APGPR | n/a | 1-5 | n/a |
| | (APGPR) | | | | |
| 19 | Enterostatin | VPGPR | n/a | 1-5 | n/a |
| | peptide seq | | | | |
| | (VPGPR) | | | | |
| 20 | Enterostatin | VPDPR | n/a | 1-5 | n/a |
| | peptide seq | | | | |
| | (VPDPR) | | | | |
| 21 | Extendin-4 | HGEGTFTSDLSKQMEEEAVRLF | n/a | 1-39 | n/a |
| | | IEWLKNGGPSSGAPPPS | | | |
| 22 | FGF-21 | MDSDETGFEHSGLWVSVLAGL | 1-28 | 29-209 | n/a |
| | | LLGACQAHPIPDSSPLLQFGGQ | | | |
| | | VRQRYLYTDDAQQTEAHLEIRE | | | |
| | | DGTVGGAADQSPESLLQLKALK | | | |
| | | PGVIQILGVKTSRFLCQRPDGAL | | | |
| | | YGSLHFDPEACSFRELLLEDGYN | | | |
| | | VYQSEAHGLPLHLPGNKSPHRD | | | |
| | | PAPRGPARFLPLPGLPPALPEPP | | | |
| | | GILAPQPPDVGSSDPLSMVGPS | | | |
| | | QGRSPSYAS | | | |
| 23 | Ghrelin | MPSPGTVCSLLLLGMLWLDLA | 1-23 | 24-36 | n/a |
| | | MAGSSFLSPEHQRVQ | | | |
| 24 | Glicentin (AA21-29) | RSLQDTEEK | n/a | 1-9 | n/a |
| 25 | Glicentin-related | RSLQDTEEKSRSFSASQADPLSD | n/a | 1-30 | n/a |
| | pancreatic | PDQMNED | | | |
| | polypeptide | | | | |
| | (AA21-50) | | | | |
| 26 | GLP-1R | MAGAPGPLRLALLLLGMVGRA | 1-23 | 24-463 | n/a |
| | | GPRPQGATVSLWETVQKWRE | | | |
| | | YRRQCQRSLTEDPPPATDLFCN | | | |
| | | RTFDEYACWPDGEPGSFVNVS | | | |
| | | CPWYLPWASSVPQGHVYRFCT | | | |
| | | AEGLWLQKDNSSLPWRDLSEC | | | |
| | | EESKRGERSSPEEQLLFLYIIYTV | | | |
| | | GYALSFSALVIASAILLGFRHLHC | | | |
| | | TRNYIHLNLFASFILRALSVFIKD | | | |
| | | AALKWMYSTAAQQHQWDGL | | | |
| | | LSYQDSLSCRLVFLLMQYCVAA | | | |
| | | NYYWLLVEGVYLYTLLAFSVLSE | | | |
| | | QWIFRLYVSIGWGVPLLFVVP | | | |
| | | WGIVKYLYEDEGCWTRNSNM | | | |
| | | NYWLIIRLPILFAIGVNFLIFVRVI | | | |
| | | CIVVSKLKANLMCKTDIKCRLAK | | | |
| | | STLTLIPLLGTHEVIFAFVMDEH | | | |
| | | ARGTLRFIKLFTELSFTSFQGLM | | | |
| | | VAILYCFVNNEVQLEFRKSWER | | | |
| | | WRLEHLHIQRDSSMKPLKCPTS | | | |
| | | SLSSGATAGSSMYTATCQASCS | | | |
| 27 | Glucagon (53-81) | HSQGTFTSDYSKYLDSRRAQDF | n/a | 1-29 | n/a |
| | | VQWLMNT | | | |
| 28 | Glucagon-like | HADGSFSDEMNTILDNLAARD | n/a | 1-33 | n/a |
| | peptide 2 (GLP-2, | FINWLIQTKITD | | | |
| | 146-178) | | | | |
| 29 | Growth hormone- | MPLWVFFFVILTLSNSSHCSPPP | 1-20 | 32-75 | 21-31; |
| | releasing hormone | PLTLRMRRYADAIFTNSYRKVL | | | 78-108 |
| | (GHRH) | GQLSARKLLQDIMSRQQGESN | | | |
| | | QERGARARLGRQVDSMWAEQ | | | |
| | | KQMELESILVALLQKHSRNSQG | | | |
| 30 | Insulin | MALWMRLLPLLALLALWGPDP | 1-24 | 25-110 | n/a |
| | | AAAFVNQHLCGSHLVEALYLVC | | | |
| | | GERGFFYTPKTRREAEDLQVGQ | | | |
| | | VELGGGPGAGSLQPLALEGSLQ | | | |
| | | KRGIVEQCCTSICSLYQLENYCN | | | |
| 31 | KBP-088 | CSNLSTCMLGRLSQELHRLQTF | n/a | 1-32 | n/a |
| | | PKTDVGANAP | | | |
| 32 | KBP-089 | CSNLSTCMLGRLSQDLHRLQTY | n/a | 1-32 | n/a |
| | | PKTDVGANAP | | | |
| 33 | Macrophage | MPGQELRTVNGSQMLLVLLVL | 1-29 | 195-308 | 30-194 |
| | inhibitory cytokine | SWLPHGGALSLAEASRASFPGP | | | |
| | 1 (MIC1 or GDF15) | SELHSEDSRFRELRKRYEDLLTRL | | | |
| | | RANQSWEDSNTDLVPAPAVRI | | | |
| | | LTPEVRLGSGGHLHLRISRAALP | | | |
| | | EGLPEASRLHRALFRLSPTASRS | | | |
| | | WDVTRPLRRQLSLARPQAPAL | | | |
| | | HLRLSPPPSQSDQLLAESSSARP | | | |
| | | QLELHLRPQAARGRRRARARN | | | |
| | | GDHCPLGPGRCCRLHTVRASLE | | | |
| | | DLGWADWVLSPREVQVTMCI | | | |
| | | GACPSQFRAANMHAQIKTSLH | | | |
| | | RLKPDTVPAPCCVPASYNPMVL | | | |
| | | IQKTDTGVSLQTYDDLLAKDCH | | | |
| | | CI | | | |
| 34 | Metreleptin | MVPIQKVQDDTKTLIKTIVTRIN | n/a | 1-147 | n/a |
| | | DISHTQSVSSKQKVTGLDFIPGL | | | |
| | | HPILTLSKMDQTLAVYQQILTS | | | |
| | | MPSRNVIQISNDLENLRDLLHV | | | |
| | | LAFSKSCHLPWASGLETLDSLG | | | |
| | | GVLEASGYSTEVVALSRLQGSL | | | |
| | | QDMLWQLDLSPGC | | | |
| 35 | Neuropeptide Y | MLGNKRLGLSGLTLALSLLVCLG | 1-28 | 29-97 | n/a |
| | (NPY) | ALAEAYPSKPDNPGEDAPAED | | | |
| | | MARYYSALRHYINLITRQRYGK | | | |
| | | RSSPETLISDLLMRESTENVPRT | | | |
| | | RLEDPAMW | | | |
| 36 | Orexin A | MNLPSTKVSWAAVTLLLLLLLLP | 1-33 | 33-66 = | 67-68; |
| | | PALLSSGAAAQPLPDCCRQKTC | | orexin A; | 98-131 |
| | | SCRLYELLHGAGNHAAGILTLG | | 69-96 = | |
| | | KRRSGPPGLQGRLQRLLQASG | | orexin B | |
| | | NHAAGILTMGRRAGAEPAPRP | | | |
| | | CLGRRCSAPAAASVAPGGQSGI | | | |
| 37 | Peptide YY (PYY3- | IKPEAPREDASPEELNRYYASLR | n/a | 1-34 | n/a |
| | 36) | HYLNLVTRQRY | | | |
| 38 | Pramlintide | KCNTATCATQRLANFLVHSSNN | n/a | 1-37 | n/a |
| | | FGPILPPTNVGSNTY | | | |
| 39 | Signal peptide | MKSIYFVAGLFVMLVQGSWQ | 1-20 | n/a | n/a |
| | (AA1-20 of | | | | |
| | preproglucagon) | | | | |
| 40 | mature pancreatic | APLEPVYPGDNATPEQMAQYA | n/a | 1-36 | n/a |
| | polypeptide | ADLRRYINMLTRPRY | | | |
| 41 | Uncoupling | GGLTASDVHPTLGVQLFSAGIA | n/a | 1-306 | n/a |
| | Protein 1 (UCP1) | ACLADVITFPLDTAKVRLQVQG | | | |
| | | ECPTSSVIRYKGVLGTITAVVKT | | | |
| | | EGRMKLYSGLPAGLQRQISSAS | | | |
| | | LRIGLYDTVQEFLTAGKETAPSL | | | |
| | | GSKILAGLTTGGVAVFIGQPTEV | | | |
| | | VKVRLQAQSHLHGIKPRYTGTY | | | |
| | | NAYRIIATTEGLTGLWKGTTPNL | | | |
| | | MRSVIINCTELVTYDLMKEAFV | | | |
| | | KNNILADDVPCHLVSALIAGFC | | | |
| | | ATAMSSPVDVVKTRFINSPPGQ | | | |
| | | YKSVPNCAMKVFTNEGPTAFFK | | | |
| | | GLVPSFLRLGSWNVIMFVCFEQ | | | |
| | | LKRELSKSRQTMDCAT | | | |
| | | See also SEQ ID NO:46, | | | |
| | | which includes M at residue 1 | | | |
| 43 | Cagrilintide | KCNTATCATQ RLAEFLRHSS | 1-21 | 49-118 | 22-48; |
| | | NNFGPILPPT NVGSNTP | | | 119-195 |

Since modifications will be apparent to those of skill in the art, it is intended that inventions herein only are limited by the scope of the appended claims.

1. A method for treating obesity, comprising administering to a subject a combination or combinations of at least three different drugs, wherein:
each drug targets a different pathway or a different target for intervention for treatment of obesity; and
the method comprises or further comprises administering a drug that increases growth hormone and/or promotes or results in muscle enhancement to counter the loss of muscle that accompanies administration of one or more of the drugs.

2. The method of claim 1, comprising one or more drugs that inhibit(s) gastric emptying selected from among one or more of a glucagon-like peptide-1 (GLP-1) or a GLP-1 receptor agonist, amylin, and pancreatic polypeptide therapeutic.

3. The method of claim 1, comprising administering a combination or combinations of drugs whose effects or activities mimic one or more of the biological effects of bariatric surgery.

4. The method of claim 3, wherein biological effects of bariatric surgery comprise reduced absorption and/or malabsorption of food, decreased appetite, increased satiety, increased glycogenolysis and/or lipolysis, increased insulin sensitivity, modulation of energy expenditure, and inhibition of gastric emptying.

5. The method of claim 1, comprising the muscle enhancing drug and combinations with two or more drugs with different activities, wherein the activities are selected from among inhibition of gastric emptying, enhancing satiety, increasing insulin release/sensitivity, modulation of energy expenditure, and smooth intestinal muscle relaxation.

6. The method of claim 1, wherein the drugs comprise or further comprise one or more of: phentermine, topiramate, metformin, empagliflozin, dapagliflozin, canagliflozin, Tesofensine (NS-2330), liothyronine, diethylpropion, levothyroxine, naltrexone, orlistat, and testosterone.

7. The method of claim 1, wherein:
the combinations of drugs are rotated;
a combination is administered for a pre-determined time, and then is replaced with a different combination; and
a rotation comprises at least two different combinations.

8. The method of claim 7, wherein the drugs in the combinations are selected from among:
a) glucagon-like peptide-1 (GLP-1) or a GLP-1 receptor agonist, Adiponectin, leptin, oxyntomodulin, peptide tyrosine-tyrosine (PYY), amylin, pancreatic peptide, enterostatin/gastric inhibitory polypeptide (GIP), cholecystokinin (CCK), vasoactive intestinal peptide (VIP), glicentin, human growth hormone or an active portion thereof or an analog of human growth hormone or an active portion thereof, ephedrine, caffeine, aspirin (ECA), oxyntomodulin, neuropeptide Y (NPY), antimicrobial peptide 2 (LEAP2), vaccine CYT009-GhrQb, the peptide-binding compound Nox-B11, and the ghrelin analog AZP-531;
b) glucagon-like peptide-1 (GLP-1) or GLP-1 receptor agonist, an appetite suppressant, a thyroid hormone, a carbonic anhydrase inhibitor, an alpha-glucosidase inhibitor, a dipeptidyl peptidase-R (DPP-4) inhibitor, a sodium-glucose co-transporter 2 (SGLT2) inhibitor, a muscle enhancer, drugs that modulate energy expenditure, a GLP-agonist, drugs that increase gastric inhibitory polypeptide (GIP), drugs that modulate GIP2, and mitochondrial uncouplers;
c) dulaglutide, bydureon, semaglutide, exenatide, liraglutide, phentermine, liothyronine, topiramate (carbonic anhydrase inhibitor), acarbose (alpha-glucosidase inhibitor), sitagliptin (dipeptidyl peptidase-4 (DPP-4) inhibitor), canagliflozin (sodium-glucose co-transporter 2 (SGLT2) inhibitor), dapagliflozin (SGLT2 inhibitor), sermorelin, mirabegron (beta-3 adrenergic agonist), and amylin; and
d) glucagon-like peptide-1 (GLP-1) or a GLP-1 receptor agonist, phentermine, thyroid hormone, carbonic anhydrase inhibitor, carbonic anhydrase inhibitor, alpha-glucosidase inhibitor, DPP-4 inhibitor, SGL2 inhibitor, muscle enhancer, and an appetite suppressant.

9. The method of claim 1, wherein the muscle enhancing drug is administered once daily, and the other drugs or combinations are selected from the following combinations:
a) GLP1, Amylin, and Pancreatic Polypeptide Therapeutic;
b) GLP1, Leptin, PYY, Amylin, Enterostatin/gastric inhibitory peptide (GIP), Cholecystokinin (CCK), and Glicentin;
c) GLP1 and Adiponectin;
d) Leptin, oxyntomodulin, and Glicentin; and
e) vasoactive intestinal peptide (VIP), wherein the muscle enhancing drug is sermorelin, tesamorelin, or IGF-1.

10. The method of claim 1, comprising rotating different combinations of drugs by:
selecting combinations of drugs, wherein the combinations of drugs are administered for a predetermined time of at least 1 week followed by administration of a different combination of drugs for a second predetermined time of at least a week, until all selected combinations of drugs are administered to complete a cycle;
up to three combinations of drugs are selected; and
repeating the same or a different cycle of combinations of drugs.

11. The method of claim 10, wherein each combination is administered for at least 1 week, or at least 2 weeks, or at least 3 weeks, or at least 4 weeks, or at least one month, or at least two months, or at least 3 months.

12. The method of claim 10, wherein each combination is administered for the same predetermined length of time, or each combination is administered for a different length of time, or at least one of the combinations is administered for a different length of time from the other combinations.

13. The method or regimen of claim 1, wherein combinations of drugs are administered for a predetermined time and the combinations are rotated over time.

14. The method or regimen of claim 13, wherein:
different combinations of drugs are administered in the morning and evening; and
the combinations of drugs are rotated every three months for at least 9 months.

15. The method of claim 1, wherein the combination(s) of drugs comprise at least three drugs and are selected from among:
a drug that inhibits gastric emptying selected from among one or more of glucagon-like peptide (GLP-1), Amylin, and Pancreatic Polypeptide Therapeutic;
a drug that enhances satiety comprising one or more drugs selected from among glucagon-like peptide-1 (GLP-1), peptide YY (PYY), amylin, enterostatin/gastric inhibitory peptide (GIP), cholecystokinin (CCK), and glicentin;
a drug that increases insulin release and/or sensitivity comprising one or both of glucagon-like peptide-1 (GLP-1) and adiponectin;
a drug that modulates energy expenditure comprising a drug selected from among leptin, oxyntomodulin, and glicentin;
a drug that results in muscle enhancement comprising one or more of sermorelin, tesamorelin and/or growth hormone, and testosterone; and
a drug that promotes intestinal smooth muscle relaxation comprising vasoactive intestinal peptide (VIP).

16. The method of claim 1, wherein the drugs are selected from among drugs that have activities or effects selected from among: drugs that inhibit gastric motility, increase insulin sensitivity, accelerate glycogenolysis and/or lipolysis, reduce eating or appetite, inhibit gastric acid secretion, limit or decrease the rate of gastric emptying, enhance muscles, increase glycogenolysis, increase insulin sensitivity, enhance the body weight-lowering and/or glucose-lowering efficacy of GLP-1, medication(s) or a therapy that decreases ghrelin or ghrelin-associated activation pathways, and drugs and treatments that reduce or antagonize ghrelin.

17. A combination of drugs for treating obesity, comprising a combination or combinations of at least three different drugs, wherein:
each drug targets a different pathway or different target for intervention for treatment of obesity; and
at least one drug increases growth hormone and/or promotes or results in muscle enhancement to eliminate or reduce the loss of muscle that accompanies administration of one or more of the drugs.

18. The combination of claim 17, comprising administering a combination or combinations of drugs whose effects or activities mimic the biological effects of bariatric surgery.

19. The combination of claim 18, wherein biological effects of bariatric surgery comprise reduced absorption and/or malabsorption of food, decreased appetite, increased satiety, increased glycogenolysis and/or lipolysis, increased insulin sensitivity, modulation of energy expenditure, and inhibition of gastric emptying.

20. The combination of claim 17, wherein the activity of the drugs comprises more than one of inhibiting gastric emptying, enhancing satiety, increasing insulin release/sensitivity; modulating energy expenditure; promoting intestinal smooth muscle relaxation; and muscle enhancement.

21. The combination of claim 17, wherein at least one drug is for muscle enhancement, and the other drugs are selected from among:
a drug for inhibiting gastric emptying is selected from among glucagon-like peptide-1 (GLP-1) or a GLP-1 receptor agonist, amylin, and pancreatic polypeptide therapeutic;
a drug for enhancing satiety is selected from among GLP-1, Leptin, PYY, Amylin, Enterostatin/gastric inhibitory peptide (GIP), Cholecystokinin (CCK), and Glicentin;
a drug for increasing insulin release/sensitivity is GLP-1 or adiponectin;
a drug for modulating energy expenditure is selected from among leptin, oxyntomodulin, and glicentin;
a drug for promoting intestinal smooth muscle relaxation is vasoactive intestinal peptide (VIP); and
the drug for muscle enhancement is sermorelin or tesamorelin.

22. A drug combination for treating obesity, comprising:
a combination or combinations of drugs or drugs whose combined effects mimic bariatric surgery, wherein each combination comprises at least three different drugs that target a different pathway or intervention target involved in the etiology of obesity;
additional drugs that promote or result in weight loss; and
one drug that enhances muscle to reduce or eliminate muscle loss that accompanies weight loss.

23. A combination of drugs, wherein the combination(s) of drugs comprise at least three drugs selected from among:
a drug that inhibits gastric emptying selected from among one or more of a glucagon-like peptide-1 (GLP-1) or a GLP1 receptor agonist, amylin, and pancreatic polypeptide therapeutic;
a drug that enhances satiety comprising one or more drugs selected from among glucagon-like peptide-1 (GLP-1), peptide YY (PYY), amylin, enterostatin/gastric inhibitory peptide (GIP), cholecystokinin (CCK), and glicentin;
a drug that increases insulin release and/or sensitivity comprising one or both of glucagon-like peptide-1 (GLP-1) and adiponectin;
a drug that modulates energy expenditure comprising a drug selected from among leptin, oxyntomodulin, and glicentin;
a drug that results in muscle enhancement comprising one or more of sermorelin, tesamorelin and/or growth hormone, and testosterone; and
a drug that promotes intestinal smooth muscle relaxation comprising vasoactive intestinal peptide (VIP),
whereby the combination of drugs results in weight loss without substantial loss of muscle.

24. The combination of claim 17, wherein the drugs are selected from among drugs that have activities or effects selected from among: drugs that inhibit gastric motility, increase insulin sensitivity, accelerate glycogenolysis and/or lipolysis, reduce eating or appetite, inhibit gastric acid secretion, limit or decrease the rate of gastric emptying, enhance muscles, increase glycogenolysis, increase insulin sensitivity, enhance the body weight-lowering and/or glucose-lowering efficacy of glucagon-like peptide-1 (GLP-1) or a GLP-1 receptor agonist, medication(s) that decrease ghrelin or ghrelin-associated activation pathways, and drugs and treatments that reduce or antagonize ghrelin.

25. The combination of claim 17, wherein the combinations of drugs are selected from among glucagon-like peptide-1 (GLP-1) or a GLP-1 agonist, adiponectin, leptin, oxyntomodulin, peptide tyrosine-tyrosine (PYY), amylin, pancreatic peptide, enterostatin/gastric inhibitory polypeptide (GIP), cholecystokinin (CCK), vasoactive intestinal peptide (VIP), glicentin, human growth hormone or an active portion thereof or an analog of human growth hormone or an active portion thereof, ephedrine, caffeine, aspirin (ECA), oxyntomodulin, neuropeptide Y (NPY), antimicrobial peptide 2 (LEAP2), vaccine CYT009-GhrQb, the peptide-binding compound Nox-B11, and the ghrelin analog AZP-531 (SEQ ID NO:15).

26. The regimen or combination of claim 17, wherein the combination of drugs comprise the a glucagon-like peptide-1 (GLP-1) or a GLP-1 receptor agonist, and one or more of an appetite suppressant, a thyroid hormone, a carbonic anhydrase inhibitor, an alpha-glucosidase inhibitor, a dipeptidyl peptidase-R (DPP-4) inhibitor, a sodium-glucose co-transporter 2 (SGLT2) inhibitor, drugs that modulate energy expenditure, a drug that increases gastric inhibitory polypeptide (GIP), a drug that modulates GIP2, and a mitochondrial uncoupler.

27. The combination of claim 17, wherein the drugs are selected from among: dulaglutide, bydureon, semaglutide, exenatide, liraglutide, phentermine, liothyronine, topiramate (carbonic anhydrase inhibitor), acarbose (alpha-glucosidase inhibitor), sitagliptin (dipeptidyl peptidase-4 (DPP-4) inhibitor), canagliflozin (sodium-glucose co-transporter 2 (SGLT2) inhibitor), dapagliflozin (SGLT2 inhibitor), sermorelin, mirabegron (beta-3 adrenergic agonist), and amylin.

28. The combination of claim 17, wherein drugs in a combination or the combinations are selected from among a GLP-1 peptide or a GLP-1 receptor agonist, phentermine, thyroid hormone, carbonic anhydrase inhibitor, carbonic anhydrase inhibitor, alpha-glucosidase inhibitor, DPP-4 inhibitor, SGL2 inhibitor, muscle enhancer, and an appetite suppressant.

29. The combination of claim 26, wherein the mitochondrial uncoupler is selected from among uncoupling protein 1 (UCP1), a catecholamine, and a small molecule uncoupler, such as 2,4-dinitrophenol (DNP) and BAM15 (N5,N6-bis(2-Fluorophenyl)[1,2,5]oxadiazolo[3,4-b]pyrazine-5,6-diamine).

30. The combination of claim 17 that comprises the muscle enhancer and one or more of a drug that inhibits gastric emptying selected from among one or more of a glucagon-like peptide-1 (GLP-1) or a GLP1 agonist, amylin, and pancreatic polypeptide therapeutic, wherein the muscle enhancer is sermorelin, tesamorelin, growth hormone, or IGF-1.

31. The combination of claim 17, wherein the drugs in the combination are in a single composition.

32. A container containing the combination of claim 31.

33. The container of claim 32 that is a syringe.


SEQUENCE LISTING (43 sequences)

SEQ ID NO: 1 - protein, 37 aa, Homo sapiens
  REGION 1..37: note = Glucagon-like peptide-1 (GLP-1)(92-128)
HDEFERHAEGTFTSDVSSYLEGQAAKEFIAWLVKGRG

SEQ ID NO: 2 - protein, 244 aa, Homo sapiens
  REGION 1..244: note = Adiponectin
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 167 aa, Homo sapiens
  REGION 1..167: note = Leptin
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 4 - protein, 37 aa, Homo sapiens
  REGION 1..37: note = Oxyntomodulin (OXY/OXM 53-89)
HSQGTFTSDYSKYLDSRRAQDFVQWLMNTKRNRNNIA

SEQ ID NO: 5 - protein, 29 aa, Homo sapiens
  REGION 1..29: note = Sermorelin
YADAXFXNSYRKVLGQLSARKLLQDXMSR

SEQ ID NO: 6 - protein, 97 aa, Homo sapiens
  REGION 1..97: note = Peptide tyrosine-tyrosine (PYY)
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 7 - protein, 37 aa, Homo sapiens [sequence omitted]

SEQ ID NO: 8 - protein, 43 aa, synthetic construct
  REGION 1..43: note = Tesamorelin
YADAIFTNSYRKLGQLSARKLLQDIMSRQQGESNQERGARARL

SEQ ID NO: 9 - protein, 105 aa, Homo sapiens
  REGION 1..105: note = Pancreatic polypeptide
MAAARLCLSLLLLSTCVALLLQPLLGAQGAPLEPVYPGDNATPEQMAQYAADLRRYINMLTRPSACPCCLFPPRYGKRHKEDTLAFSEWGSPHAAVPRELSPLDL

SEQ ID NO: 10 - protein, 153 aa, Homo sapiens
  REGION 1..153: note = gastric inhibitory polypeptide (GIP)
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 11 - protein, 115 aa, Homo sapiens
  REGION 1..115: note = cholecystokinin (CCK)
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 12 - protein, 170 aa, Homo sapiens
  REGION 1..170: note = Vasoactive intestinal peptide
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 13 - protein, 180 aa, Homo sapiens
  REGION 1..180: note = Glicentin
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 14 - protein, 132 aa, Homo sapiens
  REGION 1..132: note = AGRP (agouti-related peptide)
MLTAAVLSCALLLALPATRGAQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 15 - protein, 8 aa, synthetic construct
  REGION 1..8: note = AZP-531
RVQSPEHQ

SEQ ID NO: 16 - protein, 116 aa, Homo sapiens
  REGION 1..116: note = CAT (cocaine and amphetamine-regulated transcript, encoded by CARTPT gene)
MESSRVRLLPLLGAALLLMLPLLGTRAQEDAELQPRALDIYSAVDDASHEKELIEALQEVLKKLKSKRVPIYEKKYGQVPMCDAGEQCAVRKGARIGKLCDCPRGTSCNSFLLKCL

SEQ ID NO: 17 - protein, 32 aa, synthetic construct
  REGION 1..32: note = Davalintide (AC2307)
KCNTATCVLGRLSQELHRLQTYPRTNTGSNTY

SEQ ID NO: 18 - protein, 5 aa, Homo sapiens
  REGION 1..5: note = Enterostatin (APGPR)
APGPR

SEQ ID NO: 19 - protein, 5 aa, Homo sapiens
  REGION 1..5: note = Enterostatin peptide seq (VPGPR)
VPGPR

SEQ ID NO: 20 - protein, 5 aa, Homo sapiens
  REGION 1..5: note = Enterostatin peptide seq (VPDPR)
VPDPR

SEQ ID NO: 21 - protein, 39 aa, synthetic construct
  REGION 1..39: note = Extendin-4
HGEGTFTSDLSKQMEEEAVRLFIEWLKNGGPSSGAPPPS

SEQ ID NO: 22 - protein, 209 aa, Homo sapiens
  REGION 1..209: note = FGF-21
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 23 - protein, 36 aa, Homo sapiens
  REGION 1..36: note = Ghrelin
MPSPGTVCSLLLLGMLWLDLAMAGSSFLSPEHQRVQ

SEQ ID NO: 24 - protein, 9 aa, Homo sapiens
  REGION 1..9: note = Glicentin (AA21-29)
RSLQDTEEK

SEQ ID NO: 25 - protein, 30 aa, Homo sapiens
  REGION 1..30: note = Glicentin-related pancreatic polypeptide (AA21-50)
RSLQDTEEKSRSFSASQADPLSDPDQMNED

SEQ ID NO: 26 - protein, 463 aa, Homo sapiens
  REGION 1..463: note = GLP-1R
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 27 - protein, 29 aa, Homo sapiens
  REGION 1..29: note = Glucagon(53-81)
HSQGTFTSDYSKYLDSRRAQDFVQWLMNT

SEQ ID NO: 28 - protein, 33 aa, Homo sapiens
  REGION 1..33: note = Glucagon-like peptide2 (GLP-2, 146-178)
HADGSFSDEMNTILDNLAARDFINWLIQTKITD

SEQ ID NO: 29 - protein, 108 aa, Homo sapiens
  REGION 1..108: note = Growth hormone-releasing hormone (GHRH)
MPLWVFFFVILTLSNSSHCSPPPPLTLRMRRYADAIFTNSYRKVLGQLSARKLLQDIMSRQQGESNQERGARARLGRQVDSMWAEQKQMELESILVALLQKHSRNSQG

SEQ ID NO: 30 - protein, 110 aa, Homo sapiens
  REGION 1..110: note = insulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 31 - protein, 32 aa, synthetic construct
  REGION 1..32: note = KBP-088
CSNLSTCMLGRLSQELHRLQTFPKTDVGANAP

SEQ ID NO: 32 - protein, 32 aa, synthetic construct
  REGION 1..32: note = KBP-089
CSNLSTCMLGRLSQDLHRLQTYPKTDVGANAP

SEQ ID NO: 33 - protein, 308 aa, Homo sapiens
  REGION 1..308: note = Macrophage inhibitory cytokine 1 (MIC1 or GDF15)
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 34 - protein, 147 aa, Homo sapiens
  REGION 1..147: note = Metreleptin
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 35 - protein, 97 aa, Homo sapiens
  REGION 1..97: note = Neuropeptide Y (NPY)
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 36 - protein, 131 aa, Homo sapiens
  REGION 1..131: note = Orexin A
MNLPSTKVSWAAVTLLLLLLLLPPALLSSGAAAQPLPDCCRQKTCSCRLYELLHGAGNHAAGILTLGKRRSGPPGLQGRLQRLLQASGNHAAGILTMGRRAGAEPAPRPCLGRRCSAPAAASVAPGGQSGI

SEQ ID NO: 37 - protein, 34 aa, synthetic construct
  REGION 1..34: note = Peptide YY (PYY3-36)
IKPEAPREDASPEELNRYYASLRHYLNLVTRQRY

SEQ ID NO: 38 - protein, 37 aa, synthetic construct
  REGION 1..37: note = Pramlintide
KCNTATCATQRLANFLVHSSNNFGPILPPTNVGSNTY

SEQ ID NO: 39 - protein, 20 aa, Homo sapiens
  REGION 1..20: note = Signal peptide (AA1-20 of preproglucagon)
MKSIYFVAGLFVMLVQGSWQ

SEQ ID NO: 40 - protein, 36 aa, Homo sapiens
  REGION 1..36: note = mature pancreatic polypeptide
APLEPVYPGDNATPEQMAQYAADLRRYINMLTRPRY

SEQ ID NO: 41 - protein, 306 aa, Homo sapiens
  REGION 1..306: note = Uncoupling Protein 1 (UCP1)
GGLTASDVHPTLGVQLFSAGIAACLADVITFPLDTAKVRLQVQGECPTSSVIRYKGVLGTITAVVKTEGRMKLYSGLPAGLQRQISSASLRIGLYDTVQEFLTAGKETAPSLGSKILAGLTTGGVAVFIGQPTEVVKVRLQAQSHLHGIKPRYTGTYNAYRIIATTEGLTGLWKGTTPNLMRSVIINCTELVTYDLMKEAFVKNNILADDVPCHLVSALIAGFCATAMSSPVDVVKTRFINSPPGQYKSVPNCAMKVFTNEGPTAFFKGLVPSFLRLGSWNVIMFVCFEQLKRELSKSRQTMDCAT

SEQ ID NO: 42 - protein, 32 aa, synthetic construct
  REGION 1..32: note = KBP-042
CSNLSTCVLGKLSQELHKLQTYPRTDVGANAP

SEQ ID NO: 43 - protein, 37 aa, synthetic construct
  REGION 1..37: note = Cagrilintide
KCNTATCATQRLAEFLRHSSNNFGPILPPTNVGSNTP